(19) United States
(12) Patent Application Publication
(10) Pub. No.: US 2024/0100105 A1
(43) Pub. Date: Mar. 28, 2024

(54) BACTERIOPHAGE TREATMENT FOR ACNE AND BIOFILMS

(71) Applicants: BIOMX LTD., Nes Ziona (IL); YEDA RESEARCH AND DEVELOPMENT CO. LTD., Rehobot (IL)

(72) Inventors: Eyal Weinstock, Kfar Shmaryahu (IL); Hava Ben-David, Rehovot (IL); Kobi Konstantin Sudakov, Modi'in Makabim-Re'ut (IL); Ayelet Moses, Rishon-LeZion (IL); Sarah Rachel Pollock, Tel-Aviv (IL); Rotem Sorek, Rehovot (IL); Naomi Bluma Zak, Jerusalem (IL)

(21) Appl. No.: 18/481,324

(22) Filed: Oct. 5, 2023

Related U.S. Application Data
(63) Continuation of application No. 16/770,432, filed on Jun. 5, 2020.
(60) Provisional application No. 62/594,875, filed on Dec. 5, 2017.

(51) Int. Cl. A61K 35/76; A61K 9/00; A61K 45/06; A61P 17/10; A61P 31/04

(52) U.S. Cl. CPC A61K 35/76; A61K 9/0014; A61K 45/06; A61P 17/10; A61P 31/04; C12N 2795/00032

(57) ABSTRACT

Bacteriophage compositions and therapeutic uses thereof. In particular, compositions of lytic bacteriophages that are capable of lysing Propionibacterium acnes (P. acnes) bacterial strains associated with acne and biofilms, thereby treating or preventing acne and biofilms.

This application is a Continuation of U.S. Ser. No. 16/770,432, filed on Jun. 5, 2020 as a U.S. national stage application filed under 35 U.S.C. § 371 based on International Patent Application No. PCT/US2018/063842, filed on Dec. 4, 2018, which claims the benefit of the filing date and priority from U.S. Provisional Patent Application 62/594,875, filed on Dec. 5, 2017, the entire contents and disclosures of each of which are incorporated herein by reference.

### SEQUENCE LISTING

This instant application contains a Sequence Listing XML file which has been submitted electronically in XML format and is hereby incorporated by reference in its entirety. Said XML copy, created on Sep. 21, 2023, is named 136923-00302_SL and is 657,304 bytes in size.

### BACKGROUND OF THE DISCLOSURE

This application relates to bacteriophage compositions and therapeutic uses thereof. In a particular aspect, the disclosure relates to lytic bacteriophages that are capable of lysing Propionibacterium acnes (P. acnes) bacterial strains. In certain embodiments, the lytic bacteriophages are capable of lysing P. acnes bacterial strains that are generally thought to be associated with acne (e.g., acne vulgaris, acne conglobata, acne fulminans, Hidradenitis suppurative (acne inverse), scalp acne, acne associated with Synovitis, Acne, Pustulosis, Hyperostosis, and Osteitis (SAPHO) syndrome, acne associated with Progressive Macular Hypomelanosis and acne associated with Fatal Bacterial Granuloma after Trauma), thereby treating or preventing the disease. In certain embodiments, the lytic bacteriophages are capable of lysing P. acnes bacterial strains that are associated with biofilms, thereby treating or preventing biofilms.

Acne is one of the eight most prevalent diseases worldwide, affecting 9.4% of the global population (Tan and Bhate, 2015). In the US, −50 million people are suffering from acne, 85% of whom are at the age of 12-25 years. P. acnes is associated with the pathogenesis of acne and is thought to probably be one of the main causes of acne due to its tendency to elicit an immune response. Propionibacterium is one of the main components of human skin microbiota of healthy adults (Human Microbiome Project Consortium, 2012; Hannigan and Grice, 2013). It predominates in sebaceous regions and is estimated to represent nearly 90% of the microbiota (Fitz-Gibbon et al., 2013). The skin of teenagers suffering from acne vulgaris has up to 100-fold higher numbers of P. acnes compared to healthy skin (Leyden et al., 1975). P. acnes causes disease through a number of virulence factors, such as biofilm formation (Achermann et al., 2014). Moreover, P. acnes has also been implicated as the cause of various types of implant-associated infections (Portillo et al., 2013; Fischer et al., 2013; Perry and Lambert, 2011), including breast implants (Del Pozo et al., 2009; Rieger et al., 2009), neurosurgical shunts (Conen et al., 2008), cardiovascular devices (Delahaye et al., 2005), ocular implants (Deramo and Ting, 2001), internal fracture fixation devices, spinal hardware (Haidar et al., 2010), prosthetic joints (Piper et al., 2009), prosthetic aortic valves, prosthetic hip and shoulder implants (Stirling et al., 2001), cerebrospinal shunts (Deramo and Ting, 2001; Mayhall et al., 1984), dental infections (Delgado et al., 2011; Gribbon et al., 1994; Cove et al., 1983), elbow joint infection (Lyke et al., 2001), endocarditis (native, prosthetic valves) (Schafer et al., 2008; Guio et al., 2009), neurosurgical infections and CNS infections (Perry and Lambert 2011; McDowell et al., 2008), ocular infections (endophthalmitis, microbial keratitis) (Hunyadkurti et al., 2011; Trampuz et al., 2007), postoperative discitis, spondylodiscitis and spinal infections (Del Pozo and Patel 2009; Sampedro et al., 2010; Aucoin et al., 1986; Hoefnagel et al., 2008), prosthetic joint/orthopedic device-related infections (Zeller et al., 2007; Soderquist et al., 2010; Dodson et al., 2010; Piper et al., 2009). P. acnes can act as an opportunistic pathogen by growing as biofilms on these medical devices and other implants, and causing invasive and chronic implant infections (Achermann et al., 2014). The economic burden of implant infections is severe. For example, the annual cost of prosthetic infections in the US is projected to exceed $1.62 billion by 2020 (Kurtz S M et al., 2012).

Conventional treatment of acne typically involves the prolonged use of antibiotics, most commonly, nadifloxacin, ofloxacin, erythromycin, clindamycin hydrochloride, doxycycline, tetracycline hydrochloride, minocycline, ampicillin, cephalexin, gentamycin, and trimethoprim-sulfamethoxazole (Jońezyk-Matysiak, E, et al., 2017; Nishijima et al., 1996; Michalek et al., 2015). Prolonged antibiotic therapy in acne has led to antibiotic resistance of P. acnes strains (Sardana et al., 2016; Walsh et al., 2016) and has also been associated with the disturbance in natural microbiota of the skin and the risk of colonization of strains such as Streptococcus pyogenes (Levy et al., 2003). Experts have cautioned against excessive use of antibiotics and encourage the development of alternatives to topical antibiotic treatment for acne (Dréno, 2016). Moreover, implant-associated infections require the surgical removal of the infected implant and extensive and aggressive debridement of infected tissue, followed by prolonged antibiotic treatment, which can also lead to antibiotic resistance (Achermann et al., 2014). Hence, there is a significant unmet need for effective, reliable, and long-term treatment and/or prevention of acne and implant-associated P. acnes infections and compounds and compositions that can affect such treatment and/or prevention.

Bacteriophages are bacterial viruses that naturally control microbial populations. They are commonly found in the biosphere and therefore are environmentally friendly, because they are natural structures based on natural selection (Golkar et al., 2014). They are specific to their bacterial host and multiply at the site of infection where there are sensitive bacteria. Moreover, they have proved to be safe and well tolerated, with few side-effects or toxic effects (Jońezyk-Matysiak, E, et al. (2017).

Despite the existence of antibiotic treatments for acne, it is desirable to provide alternate treatments that avoid some of the side effects of antibiotic treatment.

### SUMMARY OF THE DISCLOSURE OF THE APPLICATION

The present application provides bacteriophage compositions and therapeutic uses thereof. In specific embodiments, the application provides lytic bacteriophages that are capable of lysing one or more P. acnes, strains, or substrains, such as P. acnes B9 strain (“B9” herein) or as P. acnes PA4 strain (“PA4” herein), or P. acnes PA3 strain (“PA3” herein), or P. acnes PA5 strain (“PA5” herein), each of which are generally thought to be associated with acne. The disclosure provides methods for treating or preventing acne on skin, preventing P. acnes biofilm development on skin and implants, or preventing implant-associated P. acnes infection. The disclosure also provides methods of selecting patients that are responsive to treatment by the methods set forth herein and patients to be treated with the methods provided herein.

As described herein the present disclosure relates to the discovery that certain bacteriophage can target and lyse the P. acnes bacteria. Accordingly, the present disclosure provides bacteriophage compositions for treating or preventing conditions associated with P. acnes colonization in/on skin, eyes, teeth and implants. The application also provides compositions comprising such bacteriophage and methods of using these compositions.

In certain aspects, the application provides a composition comprising (a) a first bacteriophage that infects and lyses at least one P. acnes strain selected from B9, PA4, PA3, and PA5; (b) a second bacteriophage that infects and lyses at least one P. acnes strain selected from B9, PA4, PA3, and PA5 that is not infected and lysed by the first bacteriophage; and (c) a pharmaceutically or cosmetically acceptable adjuvant, carrier or vehicle.

In certain aspects, the application provides a composition comprising (a) a first bacteriophage that infects and lyses at least one P. acnes strain selected from B9, PA4, PA3, and PA5; (b) a second bacteriophage that infects and lyses at least one P. acnes strain selected from B9, PA4, PA3, and PA5; (c) a third bacteriophage that infects and lyses at least one P. acnes strain selected from B9, PA4, PA3, and PA5; and (d) a pharmaceutically or cosmetically acceptable adjuvant, carrier or vehicle; wherein at least one P. acnes strain infected by the second bacteriophage is not infected and lysed by at least one of the first bacteriophage and the third bacteriophage, and wherein at least one P. acnes strain infected by the third bacteriophage is not infected and lysed by at least one of the first bacteriophage and the second bacteriophage.

In some embodiments, the composition comprises at least two, at least three, or least four or more bacteriophages selected from PS7-1, NS19-1, NS13, PA1-13, PA1-4, NS7-1, PA1-9, PA1-11, PA1-12, PA1-13, PA1-14, PA2-7, PAP-1, PAP-4, PAP-12, PAP-13, PAP-14, PA1-4, and PA2-13.

In some aspects of these embodiments, the composition comprises at least the following two bacteriophages: NS13 and NS7-1; NS13 and PA1-11; NS13 and PA1-12; NS13 and PA1-13; NS13 and PA1-14; NS13 and PA1-4; NS13 and PA1-9; NS13 and PA2-13; NS13 and PA2-7; NS13 and PAP-1; NS13 and PAP-12; NS13 and PAP-13; NS13 and PAP-14; NS13 and PAP-4; NS19-1 and NS7-1; NS19-1 and PA1-11; NS19-1 and PA1-12; NS19-1 and PA1-13; NS19-1 and PA1-14; NS19-1 and PA1-4; NS19-1 and PA1-9; NS19-1 and PA2-13; NS19-1 and PA2-7; NS19-1 and PAP-1; NS19-1 and PAP-12; NS19-1 and PAP-13; NS19-1 and PAP-14; NS19-1 and PAP-4; NS7-1 and PA1-11; NS7-1 and PA1-12; NS7-1 and PA1-13; NS7-1 and PA1-4; NS7-1 and PA1-9; NS7-1 and PA2-13; NS7-1 and PAP-12; PA1-11 and PA1-12; PA1-11 and PA-13; PA1-1 and PA2-13; PA1-1 and PAP-12; PA1-12 and PA1-13; PA1-12 and PA2-13; PA1-12 and PAP-12; PA1-13 and PA1-14; PA1-13 and PA1-4; PA1-13 and PA1-9; PA1-13 and PA2-13; PA1-13 and PA2-7; PA1-13 and PAP-1; PA1-13 and PAP-12; PA1-13 and PAP-13; PA1-13 and PAP-14; PA1-13 and PAP-4; PA1-14 and PA1-1; PA1-14 and PA1-12; PA1-14 and PA1-9; PA1-14 and PA2-13; PA1-14 and PAP-12; PA1-4 and PA1-11; PA1-4 and PA1-12; PA1-4 and PA1-14; PA1-4 and PA1-9; PA1-4 and PA2-13; PA1-4 and PA2-7; PA1-4 and PAP-1; PA1-4 and PAP-12; PA1-4 and PAP-13; PA1-4 and PAP-14; PA1-4 and PAP-4; PA1-9 and PA1-11; PA1-9 and PA1-12; PA1-9 and PA2-13; PA1-9 and PAP-12; PA2-7 and PA1-11; PA2-7 and PA1-12; PA2-7 and PA1-9; PA2-7 and PA2-13; PA2-7 and PAP-12; PAP-1 and PA1-11; PAP-1 and PA1-12; PAP-1 and PA1-9; PAP-1 and PA2-13; PAP-1 and PAP-12; PAP-12 and PA2-13; PAP-4 and PA1-11; PAP-4 and PA1-12; PAP-4 and PA1-9; PAP-4 and PA2-13; PS7-1 and NS7-1; PS7-1 and PA1-11; PS7-1 and PA1-12; PS7-1 and PA1-13; PS7-1 and PA1-14; PS7-1 and PA1-4; PS7-1 and PA1-9; PS7-1 and PA2-13; PS7-1 and PA2-7; PS7-1 and PAP-1; PS7-1 and PAP-12; PS7-1 and PAP-13; PS7-1 and PAP-14; or PS7-1 and PAP-4.

In some embodiments, the composition comprises at least the following three bacteriophages: NS13, NS7-1, and PA1-11; NS13, NS7-1, and PA1-12; NS13, NS7-1, and PA1-13; NS13, NS7-1, and PA1-4; NS13, NS7-1, and PA1-9; NS13, NS7-1, and PA2-13; NS13, NS7-1, and PAP-12; NS13, PA1-1, and PA1-12; NS13, PA1-11, and PA1-13; NS13, PA1-11, and PA1-14; NS13, PA-11, and PA1-4; NS13, PA1-11, and PA1-9; NS13, PA1-11, and PA2-13; NS13, PA1-11, and PA2-7; NS13, PA1-11, and PAP-1; NS13, PA1-11, and PAP-12; NS13, PA1-11, and PAP-13; NS13, PA1-11, and PAP-14; NS13, PA1-11, and PAP-4; NS13, PA1-12, and PA1-13; NS13, PA1-12, and PA1-14; NS13, PA1-12, and PA1-4; NS13, PA1-12, and PA1-9; NS13, PA1-12, and PA2-13; NS13, PA1-12, and PA2-7; NS13, PA1-12, and PAP-1; NS13, PA1-12, and PAP-12; NS13, PA1-12, and PAP-13; NS13, PA1-12, and PAP-14; NS13, PA1-12, and PAP-4; NS13, PA-13, and PA1-14; NS13, PA1-13, and PA1-4; NS13, PA-13, and PA1-9; NS13, PA1-13, and PA2-13; NS13, PA1-13, and PA2-7; NS13, PA1-13, and PAP-1; NS13, PA1-13, and PAP-12; NS13, PA1-13, and PAP-13; NS13, PA1-13, and PAP-14; NS13, PA1-13, and PAP-4; NS13, PA1-14, and PA1-4; NS13, PA1-14, and PA1-9; NS13, PA1-14, and PA2-13; NS13, PA1-14, and PAP-12; NS13, PA1-4, and PA1-9; NS13, PA1-4, and PA2-13; NS13, PA1-4, and PA2-7; NS13, PA1-4, and PAP-1 NS13, PA1-4, and PAP-12; NS13, PA1-4, and PAP-13; NS13, PA1-4, and PAP-14; NS13, PA1-4, and PAP-4; NS13, PA1-9, and PA2-13; NS13, PA1-9, and PA2-7; NS13, PA1-9, and PAP-1; NS13, PA1-9, and PAP-12; NS13, PA1-9, and PAP-13; NS13, PA1-9, and PAP-14; NS13, PA1-9, and PAP-4; NS13, PA2-13, and PA2-7; NS13, PA2-13, and PAP-1; NS13, PA2-13, and PAP-12; NS13, PA2-13, and PAP-13; NS13, PA2-13, and PAP-14; NS13, PA2-13, and PAP-4; NS13, PA2-7, and PAP-12; NS13, PAP-1, and PAP-12; NS13, PAP-12, and PAP-13; NS13, PAP-12, and PAP-14; NS13, PAP-12, and PAP-4; NS19-1, NS7-1, and PA1-11; NS19-1, NS7-1, and PA1-12; NS19-1, NS7-1, and PA1-13; NS19-1, NS7-1, and PA1-4; NS19-1, NS7-1, and PA1-9; NS19-1, NS7-1, and PA2-13; NS19-1, NS7-1, and PAP-12; NS19-1, PA1-11, and PA1-12; NS19-1, PA1-11, and PA1-13; NS19-1, PA1-11, and PA1-14; NS19-1, PA1-11, and PA1-4; NS19-1, PA1-11, and PA1-9; NS19-1, PA1-11, and PA2-13; NS19-1, PA1-11, and PA2-7; NS19-1, PA1-11, and PAP-1; NS19-1, PA1-11, and PAP-12; NS19-1, PA1-11, and PAP-13; NS19-1, PA1-11, and PAP-14; NS19-1, PA1-11, and PAP-4; NS19-1, PA1-12, and PA1-13; NS19-1, PA1-12, and PA1-14; NS19-1, PA1-12, and PA1-4; NS19-1, PA1-12, and PA1-9; NS19-1, PA1-12, and PA2-13; NS19-1, PA1-12, and PA2-7; NS19-1, PA1-12, and PAP-1; NS19-1, PA1-12, and PAP-12; NS19-1, PA1-12, and PAP-13; NS19-1, PA1-12, and PAP-14; NS19-1, PA1-12, and PAP-4; NS19-1, PA1-13, and PA1-14; NS19-1, PA1-13, and PA1-4; NS19-1, PA1-13, and PA1-9; NS19-1, PA1-13, and PA2-13; NS19-1, PA1-13, and PA2-7; NS19-1, PA1-13, and PAP-1; NS19-1, PA1-13, and PAP-12; NS19-1, PA1-13, and PAP-13; NS19-1, PA1-13, and PAP-14; NS19-1, PA1-13, and PAP-4; NS19-1, PA1-14, and PA1-4; NS19-1, PA1-14, and PA1-9; NS19-1, PA1-14, and PA2-13; NS19-1, PA1-14, and PAP-12; NS19-1, PA1-4, and PA1-9; NS19-1, PA1-4, and PA2-13; NS19-1, PA1-4, and PA2-7; NS19-1, PA1-4, and PAP-1; NS19-1, PA1-4, and PAP-12; NS19-1, PA1-4, and PAP-13; NS19-1, PA1-4, and PAP-14; NS19-1, PA1-4, and PAP-4; NS19-1, PA1-9, and PA2-13; NS19-1, PA1-9, and PA2-7; NS19-1, PA1-9, and PAP-1; NS19-1, PA1-9, and PAP-12; NS19-1, PA1-9, and PAP-13; NS19-1, PA1-9, and PAP-14; NS19-1, PA1-9, and PAP-4; NS19-1, PA2-13, and PA2-7; NS19-1, PA2-13, and PAP-1; NS19-1, PA2-13, and PAP-12; NS19-1, PA2-13, and PAP-13; NS19-1, PA2-13, and PAP-14; NS19-1, PA2-13, and PAP-4; NS19-1, PA2-7, and PAP-12; NS19-1, PAP-1, and PAP-12; NS19-1, PAP-12, and PAP-13; NS19-1, PAP-12, and PAP-14; NS19-1, PAP-12, and PAP-4; NS7-1, PA1-11, and PA1-13; NS7-1, PA1-11, and PA1-4; NS7-1, PA1-11, and PAP-12; NS7-1, PA1-11, and PS7-1; NS7-1, PA1-12, and PA1-13; NS7-1, PA1-12, and PA1-4; NS7-1, PA1-12, and PAP-12; NS7-1, PA1-12, and PS7-1; NS7-1, PA1-13, and PA1-4; NS7-1, PA1-13, and PA1-9; NS7-1, PA1-13, and PA2-13; NS7-1, PA1-13, and PAP-12; NS7-1, PA1-13, and PS7-1; NS7-1, PA1-4, and PA1-9; NS7-1, PA1-4, and PA2-13; NS7-1, PA1-4, and PAP-12; NS7-1, PA1-4, and PS7-1; NS7-1, PA1-9, and PAP-12; NS7-1, PA1-9, and PS7-1; NS7-1, PA2-13, and PAP-12; NS7-1, PA2-13, and PS7-1; NS7-1, PAP-12, and PS7-1; PA1-11, PA1-12, and PA1-13; PA1-11, PA1-12, and PA1-4; PA1-11, PA1-12, and PAP-12; PA1-11, PA1-12, and PS7-1; PA1-11, PA1-13, and PA1-14; PA1-11, PA1-13, and PA1-4; PA1-11, PA1-13, and PA1-9; PA1-11, PA1-13, and PA2-13; PA1-11, PA1-13, and PA2-7; PA1-11, PA1-13, and PAP-1; PA1-11, PA1-13, and PAP-12; PA1-11, PA1-13, and PAP-13; PA1-1, PA1-13, and PAP-14; PA1-11, PA1-13, and PAP-4; PA1-1, PA1-13, and PS7-1; PA1-1, PA1-14, and PA1-4; PA1-11, PA1-14, and PS7-1; PAM1-11, PA1-4, and PA1-9; PA1-1, PA1-4, and PA2-13; PA1-11, PA1-4, and PA2-7; PA1-11, PA1-4, and PAP-1; PA1-11, PA1-4, and PAP-12; PA1-11, PA1-4, and PAP-13; PA1-11, PA1-4, and PAP-14; PA1-11, PA1-4, and PAP-4; PA1-11, PA1-4, and PS7-1; PA1-11, PA1-9, and PAP-12; PA1-11, PA1-9, and PS7-1; PA1-11, PA2-13, and PAP-12; PA1-11, PA2-13, and PS7-1; PA1-11, PA2-7, and PAP-12; PA1-11, PA2-7, and PS7-1; PA1-11, PAP-1, and PAP-12; PA1-11, PAP-1, and PS7-1; PA1-11, PAP-12, and PAP-13; PA1-11, PAP-12, and PAP-14; PA1-11, PAP-12, and PAP-4; PA1-11, PAP-12, and PS7-1; PA1-11, PAP-13, and PS7-1; PA1-11, PAP-14, and PS7-1; PA1-11, PAP-4, and PS7-1; PA1-12, PA1-13, and PA1-14; PA1-12, PA1-13, and PA1-4; PA1-12, PA1-13, and PA1-9; PA1-12, PA1-13, and PA2-13; PA1-12, PA1-13, and PA2-7; PA1-12, PA1-13, and PAP-1; PA1-12, PA1-13, and PAP-12; PA1-12, PA1-13, and PAP-13; PA1-12, PA1-13, and PAP-14; PA1-12, PA1-13, and PAP-4; PA1-12, PA1-13, and PS7-1; PA1-12, PA1-14, and PA1-4; PA1-12, PA1-14, and PAP-12; PA1-12, PA1-14, and PS7-1; PA1-12, PA1-4, and PA1-9; PA1-12, PA1-4, and PA2-13; PA1-12, PA1-4, and PA2-7; PA1-12, PA1-4, and PAP-1; PA1-12, PA1-4, and PAP-12; PA1-12, PA1-4, and PAP-13; PA1-12, PA1-4, and PAP-14; PA1-12, PA1-4, and PAP-4; PA1-12, PA1-4, and PS7-1; PA1-12, PA1-9, and PAP-12; PA1-12, PA1-9, and PS7-1; PA1-12, PA2-13, and PAP-12; PA1-12, PA2-13, and PS7-1; PA1-12, PA2-7, and PAP-12; PA1-12, PA2-7, and PS7-1; PA1-12, PAP-1, and PAP-12; PA1-12, PAP-1, and PS7-1; PA1-12, PAP-12, and PAP-13; PA1-12, PAP-12, and PAP-14; PA1-12, PAP-12, and PAP-4; PA1-12, PAP-12, and PS7-1; PA1-12, PAP-13, and PS7-1; PA1-12, PAP-14, and PS7-1; PA1-12, PAP-4, and PS7-1; PA1-13, PA1-14, and PA1-4; PA1-13, PA1-14, and PA1-9; PA1-13, PA1-14, and PA2-13; PA1-13, PA1-14, and PAP-12; PA1-13, PA1-14, and PS7-1; PA1-13, PA1-4, and PA1-9; PA1-13, PA1-4, and PA2-13; PA1-13, PA1-4, and PA2-7; PA1-13, PA1-4, and PAP-1; PA1-13, PA1-4, and PAP-12; PA1-13, PA1-4, and PAP-13; PA1-13, PA1-4, and PAP-14; PA1-13, PA1-4, and PAP-4; PA1-13, PA1-4, and PS7-1; PA1-13, PA1-9, and PA2-13; PA1-13, PA1-9, and PA2-7; PA1-13, PA1-9, and PAP-1; PA1-13, PA1-9, and PAP-12; PA1-13, PA1-9, and PAP-13; PA1-13, PA1-9, and PAP-14; PA1-13, PA1-9, and PAP-4; PA1-13, PA1-9, and PS7-1; PA1-13, PA2-13, and PA2-7; PA1-13, PA2-13, and PAP-1; PA1-13, PA2-13, and PAP-12; PA1-13, PA2-13, and PAP-13; PA1-13, PA2-13, and PAP-14; PA1-13, PA2-13, and PAP-4; PA1-13, PA2-13, and PS7-1; PA1-13, PA2-7, and PAP-12; PA1-13, PA2-7, and PS7-1; PA1-13, PAP-1, and PAP-12; PA1-13, PAP-1, and PS7-1; PA1-13, PAP-12, and PAP-13; PA1-13, PAP-12, and PAP-14; PA1-13, PAP-12, and PAP-4; PA1-13, PAP-12, and PS7-1; PA1-13, PAP-13, and PS7-1; PA1-13, PAP-14, and PS7-1; PA1-13, PAP-4, and PS7-1; PA1-14, PA1-4, and PA1-9; PA1-14, PA1-4, and PA2-13; PA1-14, PA1-4, and PAP-12; PA1-14, PA1-4, and PS7-1; PA1-14, PA1-9, and PAP-12; PA1-14, PA1-9, and PS7-1; PA1-14, PA2-13, and PAP-12; PA1-14, PA2-13, and PS7-1; PA1-14, PAP-12, and PS7-1; PA1-4, PA1-9, and PA2-13; PA1-4, PA1-9, and PA2-7; PA1-4, PA1-9, and PAP-1; PA1-4, PA1-9, and PAP-12; PA1-4, PA1-9, and PAP-13; PA1-4, PA1-9, and PAP-14; PA1-4, PA1-9, and PAP-4; PA1-4, PA1-9, and PS7-1; PA1-4, PA2-13, and PA2-7; PA1-4, PA2-13, and PAP-1; PA1-4, PA2-13, and PAP-12; PA1-4, PA2-13, and PAP-13; PA1-4, PA2-13, and PAP-14; PA1-4, PA2-13, and PAP-4; PA1-4, PA2-13, and PS7-1; PA1-4, PA2-7, and PAP-12; PA1-4, PA2-7, and PS7-1; PA1-4, PAP-1, and PAP-12; PA1-4, PAP-1, and PS7-1; PA1-4, PAP-12, and PAP-13; PA1-4, PAP-12, and PAP-14; PA1-4, PAP-12, and PAP-4; PA1-4, PAP-12, and PS7-1; PA1-4, PAP-13, and PS7-1; PA1-4, PAP-14, and PS7-1; PA1-4, PAP-4, and PS7-1; PA1-9, PA2-13, and PAP-12; PA1-9, PA2-13, and PS7-1; PA1-9, PA2-7, and PAP-12; PA1-9, PA2-7, and PS7-1; PA1-9, PAP-1, and PAP-12; PA1-9, PAP-1, and PS7-1; PA1-9, PAP-12, and PAP-13; PA1-9, PAP-12, and PAP-14; PA1-9, PAP-12, and PAP-4; PA1-9, PAP-12, and PS7-1; PA1-9, PAP-13, and PS7-1; PA1-9, PAP-14, and PS7-1; PA1-9, PAP-4, and PS7-1; PA2-13, PA2-7, and PAP-12; PA2-13, PA2-7, and PS7-1; PA2-13, PAP-1, and PAP-12; PA2-13, PAP-1, and PS7-1; PA2-13, PAP-12, and PAP-13; PA2-13, PAP-12, and PAP-14; PA2-13, PAP-12, and PAP-4; PA2-13, PAP-12, and PS7-1; PA2-13, PAP-13, and PS7-1; PA2-13, PAP-14, and PS7-1; PA2-13, PAP-4, and PS7-1; PA2-7, PAP-12, and PS7-1; PAP-1, PAP-12, and PS7-1; PAP-12, PAP-13, and PS7-1; PAP-12, PAP-14, and PS7-1; or PAP-12, PAP-4, and PS7-1.

In some embodiments, the composition comprises at least the following four bacteriophages: NS13, NS7-1, PA1-11, and PA1-4; NS13, NS7-1, PA1-11, and PAP-12; NS13, NS7-1, PA1-12, and PA1-4; NS13, NS7-1, PA1-12, and PAP-12; NS13, NS7-1, PA1-13, and PA1-4; NS13, NS7-1, PA1-13, and PAP-12; NS13, NS7-1, PA1-4, and PA1-9; NS13, NS7-1, PA1-4, and PA2-13; NS13, NS7-1, PA1-4, and PS7-1; NS13, NS7-1, PA1-9, and PAP-12; NS13, NS7-1, PA2-13, and PAP-12; NS13, PA1-11, PA1-12, and PA1-4; NS13, PA1-11, PA1-12, and PAP-12; NS13, PA1-11, PA1-13, and PA1-4; NS13, PA1-11, PA1-13, and PAP-12; NS13, PA1-11, PA1-14, and PA1-4; NS13, PA1-11, PA1-14, and PAP-12; NS13, PA1-11, PA1-4, and PA1-9; NS13, PA1-11, PA1-4, and PA2-13; NS13, PA1-11, PA1-4, and PA2-7; NS13, PA1-11, PA1-4, and PAP-12; NS13, PA1-11, PA1-4, and PAP-13; NS13, PA1-11, PA1-4, and PAP-14; NS13, PA1-11, PA1-4, and PAP-4; NS13, PA1-11, PA1-4, and PS7-1; NS13, PA1-11, PA1-9, and PAP-12; NS13, PA1-11, PA2-13, and PAP-12; NS13, PA1-11, PA2-7, and PAP-12; NS13, PA1-11, PAP-12, and PAP-13; NS13, PA1-11, PAP-12, and PAP-14; NS13, PA1-11, PAP-12, and PAP-4; NS13, PA1-11, PAP-12, and PS7-1; NS13, PA1-12, PA1-13, and PA1-4; NS13, PA1-12, PA1-13, and PAP-12; NS13, PA1-12, PA1-14, and PA1-4; NS13, PA1-12, PA1-14, and PAP-12; NS13, PA1-12, PA1-4, and PA1-9; NS13, PA1-12, PA1-4, and PA2-13; NS13, PA1-12, PA1-4, and PA2-7; NS13, PA1-12, PA1-4, and PAP-12; NS13, PA1-12, PA1-4, and PAP-13; NS13, PA1-12, PA1-4, and PAP-14; NS13, PA1-12, PA1-4, and PAP-4; NS13, PA1-12, PA1-4, and PS7-1; NS13, PA1-12, PA1-9, and PAP-12; NS13, PA1-12, PA2-13, and PAP-12; NS13, PA1-12, PA2-7, and PAP-12; NS13, PA1-12, PAP-12, and PAP-13; NS13, PA1-12, PAP-12, and PAP-14; NS13, PA1-12, PAP-12, and PAP-4; NS13, PA1-12, PAP-12, and PS7-1; NS13, PA1-13, PA1-14, and PA1-4; NS13, PA1-13, PA1-14, and PAP-12; NS13, PA1-13, PA1-4, and PA1-9; NS13, PA1-13, PA1-4, and PA2-13; NS13, PA1-13, PA1-4, and PA2-7; NS13, PA1-13, PA1-4, and PAP-12; NS13, PA1-13, PA1-4, and PAP-13; NS13, PA1-13, PA1-4, and PAP-14; NS13, PA1-13, PA1-4, and PAP-4; NS13, PA1-13, PA1-4, and PS7-1; NS13, PA1-13, PA1-9, and PAP-12; NS13, PA1-13, PA2-13, and PAP-12; NS13, PA1-13, PA2-7, and PAP-12; NS13, PA1-13, PAP-12, and PAP-13; NS13, PA1-13, PAP-12, and PAP-14; NS13, PA1-13, PAP-12, and PAP-4; NS13, PA1-13, PAP-12, and PS7-1; NS13, PA1-14, PA1-4, and PA1-9; NS13, PA1-14, PA1-4, and PA2-13; NS13, PA1-14, PA1-4, and PS7-1; NS13, PA1-14, PA1-9, and PAP-12; NS13, PA1-14, PA2-13, and PAP-12; NS13, PA1-14, PAP-12, and PS7-1; NS13, PA1-4, PA1-9, and PA2-13; NS13, PA1-4, PA1-9, and PA2-7; NS13, PA1-4, PA1-9, and PAP-12; NS13, PA1-4, PA1-9, and PAP-13; NS13, PA1-4, PA1-9, and PAP-14; NS13, PA1-4, PA1-9, and PAP-4; NS13, PA1-4, PA1-9, and PS7-1; NS13, PA1-4, PA2-13, and PA2-7; NS13, PA1-4, PA2-13, and PAP-12; NS13, PA1-4, PA2-13, and PAP-13; NS13, PA1-4, PA2-13, and PAP-14; NS13, PA1-4, PA2-13, and PAP-4; NS13, PA1-4, PA2-7, and PS7-1; NS13, PA1-4, PAP-12, and PS7-1; NS13, PA1-4, PAP-13, and PS7-1; NS13, PA1-4, PAP-14, and PS7-1; NS13, PA1-4, PAP-4, and PS7-1; NS13, PA1-9, PA2-13, and PAP-12; NS13, PA1-9, PA2-7, and PAP-12; NS13, PA1-9, PAP-12, and PAP-13; NS13, PA1-9, PAP-12, and PAP-14; NS13, PA1-9, PAP-12, and PAP-4; NS13, PA1-9, PAP-12, and PS7-1; NS13, PA2-13, PA2-7, and PAP-12; NS13, PA2-13, PAP-12, and PAP-13; NS13, PA2-13, PAP-12, and PAP-14; NS13, PA2-13, PAP-12, and PAP-4; NS13, PA2-7, PAP-12, and PS7-1; NS13, PAP-12, PAP-13, and PS7-1; NS13, PAP-12, PAP-14, and PS7-1; NS13, PAP-12, PAP-4, and PS7-1; NS19-1, NS7-1, PA1-11, and PA1-4; NS19-1, NS7-1, PA1-11, and PAP-12; NS19-1, NS7-1, PA1-12, and PA1-4; NS19-1, NS7-1, PA1-12, and PAP-12; NS19-1, NS7-1, PA1-13, and PA1-4; NS19-1, NS7-1, PA1-13, and PAP-12; NS19-1, NS7-1, PA1-4, and PA1-9; NS19-1, NS7-1, PA1-4, and PA2-13; NS19-1, NS7-1, PA1-4, and PS7-1; NS19-1, NS7-1, PA1-9, and PAP-12; NS19-1, NS7-1, PA2-13, and PAP-12; NS19-1, NS7-1, PAP-12, and PS7-1; NS19-1, PA1-11, PA1-12, and PA1-4; NS19-1, PA1-11, PA1-12, and PAP-12; NS19-1, PA1-11, PA1-13, and PA1-4; NS19-1, PA1-1, PA1-13, and PAP-12; NS19-1, PA1-1, PA1-14, and PA1-4; NS19-1, PA1-11, PA1-14, and PAP-12; NS19-1, PA1-11, PA1-4, and PA1-9; NS19-1, PA1-11, PA1-4, and PA2-13; NS19-1, PA1-11, PA1-4, and PA2-7; NS19-1, PA1-11, PA1-4, and PAP-12; NS19-1, PA1-11, PA1-4, and PAP-13; NS19-1, PA1-11, PA1-4, and PAP-14; NS19-1, PA1-11, PA1-4, and PAP-4; NS19-1, PA1-11, PA1-4, and PS7-1; NS19-1, PA1-11, PA1-9, and PAP-12; NS19-1, PA1-11, PA2-13, and PAP-12; NS19-1, PA1-11, PA2-7, and PAP-12; NS19-1, PA1-11, PAP-12, and PAP-13; NS19-1, PA1-11, PAP-12, and PAP-14; NS19-1, PA1-1, PAP-12, and PAP-4; NS19-1, PA1-1, PAP-12, and PS7-1; NS19-1, PA1-12, PA1-13, and PA1-4; NS19-1, PA1-12, PA1-13, and PAP-12; NS19-1, PA1-12, PA1-14, and PA1-4; NS19-1, PA1-12, PA1-14, and PAP-12; NS19-1, PA1-12, PA1-4, and PA1-9; NS19-1, PA1-12, PA1-4, and PA2-13; NS19-1, PA1-12, PA1-4, and PA2-7; NS19-1, PA1-12, PA1-4, and PAP-12; NS19-1, PA1-12, PA1-4, and PAP-13; NS19-1, PA1-12, PA1-4, and PAP-14; NS19-1, PA1-12, PA1-4, and PAP-4; NS19-1, PA1-12, PA1-4, and PS7-1; NS19-1, PA1-12, PA1-9, and PAP-12; NS19-1, PA1-12, PA2-13, and PAP-12; NS19-1, PA1-12, PA2-7, and PAP-12; NS19-1, PA1-12, PAP-12, and PAP-13; NS19-1, PA1-12, PAP-12, and PAP-14; NS19-1, PA1-12, PAP-12, and PAP-4; NS19-1, PA1-12, PAP-12, and PS7-1; NS19-1, PA1-13, PA1-14, and PA1-4; NS19-1, PA1-13, PA1-14, and PAP-12; NS19-1, PA1-13, PA1-4, and PA1-9; NS19-1, PA1-13, PA1-4, and PA2-13; NS19-1, PA1-13, PA1-4, and PA2-7; NS19-1, PA1-13, PA1-4, and PAP-12; NS19-1, PA1-13, PA1-4, and PAP-13; NS19-1, PA1-13, PA1-4, and PAP-14; NS19-1, PA1-13, PA1-4, and PAP-4; NS19-1, PA1-13, PA1-4, and PS7-1; NS19-1, PA1-13, PA1-9, and PAP-12; NS19-1, PA1-13, PA2-13, and PAP-12; NS19-1, PA1-13, PA2-7, and PAP-12; NS19-1, PA1-13, PAP-12, and PAP-13; NS19-1, PA1-13, PAP-12, and PAP-14; NS19-1, PA1-13, PAP-12, and PAP-4; NS19-1, PA1-13, PAP-12, and PS7-1; NS19-1, PA1-14, PA1-4, and PA1-9; NS19-1, PA1-14, PA1-4, and PA2-13; NS19-1, PA1-14, PA1-4, and PS7-1; NS19-1, PA1-14, PA1-9, and PAP-12; NS19-1, PA1-14, PA2-13, and PAP-12; NS19-1, PA1-14, PAP-12, and PS7-1; NS19-1, PA1-4, PA1-9, and PA2-13; NS19-1, PA1-4, PA1-9, and PA2-7; NS19-1, PA1-4, PA1-9, and PAP-12; NS19-1, PA1-4, PA1-9, and PAP-13; NS19-1, PA1-4, PA1-9, and PAP-14; NS19-1, PA1-4, PA1-9, and PAP-4; NS19-1, PA1-4, PA1-9, and PS7-1; NS19-1, PA1-4, PA2-13, and PA2-7; NS19-1, PA1-4, PA2-13, and PAP-12; NS19-1, PA1-4, PA2-13, and PAP-13; NS19-1, PA1-4, PA2-13, and PAP-14; NS19-1, PA1-4, PA2-13, and PAP-4; NS19-1, PA1-4, PA2-7, and PS7-1; NS19-1, PA1-4, PAP-12, and PS7-1; NS19-1, PA1-4, PAP-13, and PS7-1; NS19-1, PA1-4, PAP-14, and PS7-1; NS19-1, PA1-4, PAP-4, and PS7-1; NS19-1, PA1-9, PA2-13, and PAP-12; NS19-1, PA1-9, PA2-7, and PAP-12; NS19-1, PA1-9, PAP-12, and PAP-13; NS19-1, PA1-9, PAP-12, and PAP-14; NS19-1, PA1-9, PAP-12, and PAP-4; NS19-1, PA1-9, PAP-12, and PS7-1; NS19-1, PA2-13, PA2-7, and PAP-12; NS19-1, PA2-13, PAP-12, and PAP-13; NS19-1, PA2-13, PAP-12, and PAP-14; NS19-1, PA2-13, PAP-12, and PAP-4; NS19-1, PA2-7, PAP-12, and PS7-1; NS19-1, PAP-12, PAP-13, and PS7-1; NS19-1, PAP-12, PAP-14, and PS7-1; NS19-1, PAP-12, PAP-4, and PS7-1; NS7-1, PA1-11, PA1-13, and PA1-4; NS7-1, PA1-11, PA1-13, and PAP-12; NS7-1, PA1-1, PA1-4, and PAP-12; NS7-1, PA1-11, PA1-4, and PS7-1; NS7-1, PA1-11, PAP-12, and PS7-1; NS7-1, PA1-12, PA1-13, and PA1-4; NS7-1, PA1-12, PA1-13, and PAP-12; NS7-1, PA1-12, PA1-4, and PAP-12; NS7-1, PA1-12, PA1-4, and PS7-1; NS7-1, PA1-12, PAP-12, and PS7-1; NS7-1, PA1-13, PA1-4, and PA1-9; NS7-1, PA1-13, PA1-4, and PA2-13; NS7-1, PA1-13, PA1-4, and PAP-12; NS7-1, PA1-13, PA1-4, and PS7-1; NS7-1, PA1-13, PA1-9, and PAP-12; NS7-1, PA1-13, PA2-13, and PAP-12; NS7-1, PA1-13, PAP-12, and PS7-1; NS7-1, PA1-4, PA1-9, and PAP-12; NS7-1, PA-4, PA1-9, and PS7-1; NS7-1, PA1-4, PA2-13, and PAP-12; NS7-1, PA1-4, PA2-13, and PS7-1; NS7-1, PA1-4, PAP-12, and PS7-1; NS7-1, PA1-9, PAP-12, and PS7-1; NS7-1, PA2-13, PAP-12, and PS7-1; PA1-11, PA1-12, PA1-13, and PA1-4; PA1-11, PA1-12, PA1-13, and PAP-12; PAM1-11, PA1-12, PA1-4, and PAP-12; PA1-1, PA1-12, PA1-4, and PS7-1; PA1-11, PA1-12, PAP-12, and PS7-1; PA1-11, PA1-13, PA1-14, and PA1-4; PA1-11, PA1-13, PA1-14, and PAP-12; PA1-11, PA1-13, PA1-4, and PA1-9; PA1-1, PA1-13, PA1-4, and PA2-13; PA1-1, PA1-13, PA1-4, and PA2-7; PA1-11, PA1-13, PA1-4, and PAP-12; PA1-11, PA1-13, PA1-4, and PAP-13; PA1-11, PA1-13, PA1-4, and PAP-14; PA1-11, PA1-13, PA1-4, and PAP-4; PA1-11, PA1-13, PA1-4, and PS7-1; PA1-1, PA1-13, PA1-4, and PS7-1; PA1-1, PA1-13, PA1-9, and PAP-12; PAM1-11, PA1-13, PA2-13, and PAP-12; PA1-11, PA1-13, PA2-7, and PAP-12; PA1-11, PA1-13, PAP-12, and PAP-13; PA1-11, PA1-13, PAP-12, and PAP-14; PA1-11, PA1-13, PAP-12, and PAP-4; PA1-1, PA1-13, PAP-12, and PS7-1; PA1-1, PA1-13, PAP-12, and PS7-1; PA1-1, PA1-14, PA1-4, and PAP-12; PA1-11, PA1-14, PA1-4, and PS7-1; PA1-11, PA1-14, PAP-12, and PS7-1; PA1-11, PA1-4, PA1-9, and PAP-12; PA1-11, PA1-4, PA1-9, and PS7-1; PA1-11, PA1-4, PA1-9, and PAP-12; PA1-11, PA1-4, PA2-13, and PAP-12; PA1-11, PA1-4, PA2-13, and PS7-1; PA1-11, PA1-4, PA2-7, and PAP-12; PA1-11, PA1-4, PA2-7, and PS7-1; PA1-11, PA1-4, PAP-12, and PAP-13; PA1-11, PA1-4, PAP-12, and PAP-14; PA1-11, PA1-4, PAP-12, and PAP-4; PA1-11, PA1-4, PAP-12, and PS7-1; PA1-11, PA1-4, PAP-12, and PS7-1; PA1-11, PA1-4, PAP-13, and PS7-1; PA-11, PA1-4, PAP-14, and PS7-1; PA1-11, PA1-4, PAP-4, and PS7-1; PA1-11, PA1-9, PAP-12, and PS7-1; PA1-11, PA2-13, PAP-12, and PS7-1; PA1-11, PA2-7, PAP-12, and PS7-1; PA1-1, PAP-12, PAP-13, and PS7-1; PA1-11, PAP-12, PAP-14, and PS7-1; PA1-11, PAP-12, PAP-4, and PS7-1; PA1-12, PA1-13, PA1-14, and PA1-4; PA1-12, PA1-13, PA1-14, and PAP-12; PA1-12, PA-13, PA1-4, and PA1-9; PA1-12, PA-13, PA1-4, and PA2-13; PA1-12, PA1-13, PA1-4, and PA2-7; PA1-12, PA1-13, PA1-4, and PAP-12; PA1-12, PA1-13, PA1-4, and PAP-13; PA1-12, PA1-13, PA1-4, and PAP-14; PA1-12, PA1-13, PA1-4, and PAP-4; PA1-12, PA1-13, PA1-4, and PS7-1; PA1-12, PA1-13, PA1-4, and PS7-1; PA1-12, PA1-13, PA1-9, and PAP-12; PA1-12, PA1-13, PA2-13, and PAP-12; PA1-12, PA1-13, PA2-7, and PAP-12; PA1-12, PA1-13, PAP-12, and PAP-13; PA1-12, PA1-13, PAP-12, and PAP-14; PA1-12, PA1-13, PAP-12, and PAP-4; PA1-12, PA1-13, PAP-12, and PS7-1; PA1-12, PA1-13, PAP-12, and PS7-1; PA1-12, PA1-14, PA1-4, and PAP-12; PA-12, PA1-14, PA1-4, and PS7-1; PA1-12, PA1-14, PAP-12, and PS7-1; PA1-12, PA1-4, PA1-9, and PAP-12; PA1-12, PA1-4, PA1-9, and PS7-1; PA1-12, PA1-4, PA2-13, and PS7-1; PA1-12, PA1-4, PA2-7, and PAP-12; PA1-12, PA1-4, PA2-7, and PS7-1; PA1-12, PA1-4, PAP-12, and PAP-13; PA1-12, PA1-4, PAP-12, and PAP-14; PA1-12, PA1-4, PAP-12, and PAP-4; PA1-12, PA1-4, PAP-12, and PS7-1; PA1-12, PA1-4, PAP-13, and PS7-1; PA1-12, PA1-4, PAP-14, and PS7-1; PA1-12, PA1-4, PAP-4, and PS7-1; PA1-12, PA1-9, PAP-12, and PS7-1; PA1-12, PA2-13, PAP-12, and PS7-1; PA1-12, PA2-7, PAP-12, and PS7-1; PA1-12, PAP-12, PAP-13, and PS7-1; PA1-12, PAP-12, PAP-14, and PS7-1; PA1-12, PAP-12, PAP-4, and PS7-1; PA1-13, PA1-14, PA1-4, and PA1-9; PA1-13, PA1-14, PA1-4, and PA2-13; PA1-13, PA1-14, PA1-4, and PAP-12; PA1-13, PA1-14, PA1-4, and PS7-1; PA1-13, PA1-14, PA1-9, and PAP-12; PA1-13, PA1-14, PA2-13, and PAP-12; PA1-13, PA1-14, PAP-12, and PS7-1; PA1-13, PA1-4, PA1-9, and PA2-13; PA1-13, PA1-4, PA1-9, and PA2-7; PA1-13, PA1-4, PA1-9, and PAP-12; PA1-13, PA1-4, PA1-9, and PAP-13; PA1-13, PA1-4, PA1-9, and PAP-14; PA I-13, PA1-4, PA1-9, and PAP-4; PA1-13, PA1-4, PA1-9, and PS7-1; PA1-13, PA1-4, PA1-9, and PS7-1; PA1-13, PA1-4, PA2-13, and PA2-7; PA1-13, PA1-4, PA2-13, and PAP-12; PA1-13, PA1-4, PA2-13, and PAP-13; PA1-13, PA1-4, PA2-13, and PAP-14; PA1-13, PA1-4, PA2-13, and PAP-4; PA1-13, PA1-4, PA2-13, and PS7-1; PA1-13, PA1-4, PA2-7, and PAP-12; PA1-13, PA1-4, PA2-7, and PS7-1; PA1-13, PA1-4, PAP-12, and PAP-4; PA1-13, PA1-4, PAP-12, and PS7-1; PA1-13, PA1-4, PAP-12, and PS7-1; PA1-13, PA1-4, PAP-13, and PS7-1; PA1-13, PA1-4, PAP-14, and PS7-1; PA1-13, PA-4, PAP-4, and PS7-1; PA1-13, PA1-9, PA2-13, and PAP-12; PA1-13, PA1-9, PA2-7, and PAP-12; PA1-13, PA1-9, PAP-12, and PAP-13; PA1-13, PA1-9, PAP-12, and PAP-14; PA1-13, PA1-9, PAP-12, and PAP-4; PA1-13, PA1-9, PAP-12, and PS7-1; PA1-13, PA1-9, PAP-12, and PS7-1; PA1-13, PA2-13, PA2-7, and PAP-12; PA1-13, PA2-13, PAP-12, and PAP-13; PA1-13, PA2-13, PAP-12, and PAP-14; PA1-13, PA2-13, PAP-12, and PAP-4; PA1-13, PA2-13, PAP-12, and PS7-1; PA1-13, PA2-7, PAP-12, and PS7-1; PA1-13, PAP-12, PAP-13, and PS7-1; PA1-13, PAP-12, PAP-14, and PS7-1; PA1-13, PAP-12, PAP-4, and PS7-1; PA1-14, PA1-4, PA1-9, and PAP-12; PA1-14, PA1-4, PA1-9, and PS7-1; PA1-14, PA1-4, PA2-13, and PAP-12; PA1-14, PA1-4, PA2-13, and PS7-1; PA1-14, PA1-4, PAP-12, and PS7-1; PA1-14, PA1-9, PAP-12, and PS7-1; PA1-14, PA2-13, PAP-12, and PS7-1; PA1-4, PA1-9, PA2-13, and PAP-12; PA1-4, PA1-9, PA2-13, and PS7-1; PA1-4, PA1-9, PA2-7, and PAP-12; PA1-4, PA1-9, PA2-7, and PS7-1; PA1-4, PA1-9, PAP-12, and PAP-13; PA1-4, PA1-9, PAP-12, and PAP-14; PA1-4, PA1-9, PAP-12, and PAP-4; PA1-4, PA1-9, PAP-12, and PS7-1; PA1-4, PA1-9, PAP-13, and PS7-1; PA-4, PA1-9, PAP-14, and PS7-1; PA-4, PA1-9, PAP-4, and PS7-1; PA1-4, PA2-13, PA2-7, and PAP-12; PA1-4, PA2-13, PA2-7, and PS7-1; PA1-4, PA2-13, PAP-12, and PAP-13; PA1-4, PA2-13, PAP-12, and PAP-14; PA1-4, PA2-13, PAP-12, and PAP-4; PA1-4, PA2-13, PAP-12, and PS7-1; PA1-4, PA2-13, PAP-13, and PS7-1; PA1-4, PA2-13, PAP-14, and PS7-1; PA1-4, PA2-13, PAP-4, and PS7-1; PA1-4, PA2-7, PAP-12, and PS7-1; PA1-4, PAP-12, PAP-13, and PS7-1; PA1-4, PAP-12, PAP-14, and PS7-1; PA1-4, PAP-12, PAP-4, and PS7-1; PA1-9, PA2-13, PAP-12, and PS7-1; PA1-9, PA2-7, PAP-12, and PS7-1; PA1-9, PAP-12, PAP-13, and PS7-1; PA1-9, PAP-12, PAP-14, and PS7-1; PA1-9, PAP-12, PAP-4, and PS7-1; PA2-13, PA2-7, PAP-12, and PS7-1; PA2-13, PAP-12, PAP-13, and PS7-1; PA2-13, PAP-12, PAP-14, and PS7-1; or PA2-13, PAP-12, PAP-4, and PS7-1.

In certain aspects, the application provides a composition comprising at least two bacteriophages capable of lysing one or more strains of a P. acnes bacterium, wherein the first selected phage infects and lyses P. acnes strain B9, the second selected phage infects and lyses P. acnes strain PA4, and wherein the two selected phages have different lytic specificities from one another with respect to P. acnes strains B9 and PA4.

In certain aspects, the application provides a composition comprising at least two bacteriophages capable of lysing one or more strains of a P. acnes bacterium, wherein the first selected phage infects and lyses P. acnes strain PA3, the second selected phage infects and lyses P. acnes strain B9, and wherein the two selected phages have different lytic specificities from one another with respect to P. acnes strains PA3 and B9.

In certain aspects, the application provides a composition comprising at least two bacteriophages capable of lysing one or more strains of a P. acnes bacterium, wherein the first selected phage infects and lyses P. acnes strain PA3, the second selected phage infects and lyses P. acnes strain PA5, and wherein the two selected phages have different lytic specificities from one another with respect to P. acnes strains PA3 and PA5.

In certain aspects, the application provides a composition comprising at least two bacteriophages capable of lysing one or more strains of a P. acnes bacterium, wherein the first selected phage infects and lyses P. acnes strain PA4, the second selected phage infects and lyses P. acnes strain PA5, and wherein the two selected phages have different lytic specificities from one another with respect to P. acnes strains PA4 and PA5.

In some embodiments, the one or more of the above compositions (see [0009]-[0018]) also comprise at least one phage that infects and lyses at least one P. acnes strain selected from PA1, PA2, PA6, PA7, PA8, PA9, PA10, PA11, and PAP. In some embodiments, the one or more of the above compositions comprise phages that infect and lyse each of P. acnes strains PA1, PA2, PA6, PA7, PA8, PA9, PA10, PA11, and PAP in toto.

In certain aspects, the application provides a composition comprising at least three bacteriophages capable of lysing one or more strains of a P. acnes bacterium, wherein the first selected phage infects and lyses P. acnes strain PA3, the second selected phage infects and lyses P. acnes strain PA4, and the third selected phage infects and lyses P. acnes strain B9, wherein each of the three selected phages have different lytic specificities from one another with respect to P. acnes strains PA3, PA4 and B9.

In some embodiments, the one or more of the above compositions (see [0009]-[0018]) comprises at least three bacteriophages selected from PS7-1, PA1-11, PAP-12, PA1-9 and PA1-13. In preferred embodiments, the composition comprises the following combinations of bacteriophages formulated for delivery to skin, eyes, teeth, or to an implant: PAP-12, PA1-9, and PA1-13; PS7-1, PA1-9, and PA1-13; PAP-12, PA1-9, PA1-13, and PS7-1; PA1-13, PAP-12, and PA1-11; or PS7-1, PA1-13, and PAP-12.

In some embodiments, the composition is formulated for delivery to mammalian skin, mammalian eyes, mammalian teeth or an implant to be inserted into a mammal. Preferably, the mammal is a human.

In some embodiments, the composition is formulated for topical application. In some embodiments, the composition is in the form of a gel, cream, ointment, lotion, paste, solution, microemulsion, liquid wash, spray, application stick, cosmetic, dressing, face-wash, soap, powder, spray, capsule, eye drop, eye ointment, eye lotion, solid, or a moist sponge wipe, or is bonded to a solid surface. In some embodiments, the composition comprises an adjuvant, a carrier or a vehicle. In some embodiments, the composition comprises one or more additives selected from solubilizers, emollients, humectants, thickening agents, permeation enhancers, chelating agents, antioxidants, buffering agents, isotonic agents, suspending agents, emulsifying agents, stabilizers and preservatives. In some embodiments, the composition comprises one or more of a gel-forming agent, a cream-forming agent, a wax, an oil, a surfactant, and a binder.

In certain aspects, the application provides a method for treating or preventing acne in a subject in need or at risk thereof, the method comprising administering to the subject a composition comprising (a) a first bacteriophage that infects and lyses at least one Propionibacterium acnes (P. acnes) strain selected from B9, PA4, PA3, and PA5; and (b) a second bacteriophage that infects and lyses at least one P. acnes strain selected from B9, A4, PA3, and PA5 that is not infected and lysed by the first bacteriophage. In some embodiments, the composition comprises (a) a first bacteriophage that infects and lyses at least one P. acnes strain selected from B9, PA4, PA3, and PA5; (b) a second bacteriophage that infects and lyses at least one P. acnes strain selected from B9, PA4, PA3, and PA5; and (c) a third bacteriophage that infects and lyses at least one P. acnes strain selected from B9, PA4, PA3, and PA5; wherein at least one P. acnes strain infected by the second bacteriophage is not infected and lysed by at least one of the first bacteriophage and the third bacteriophage, and wherein at least one P. acnes strain infected by the third bacteriophage is not infected and lysed by at least one of the first bacteriophage and the second bacteriophage. In some embodiments, the composition comprises (a) a first bacteriophage that infects and lyses P. acnes strain PA3; (b) a second bacteriophage that infects and lyses P. acnes strain PA4; and (c) a third bacteriophage that infects and lyses P. acnes strain B9, wherein each of the three bacteriophages have different lytic specificities from one another with respect to P. acnes strains PA3, PA4 and B9. In some embodiments, the application provides a method for treating or preventing acne in a subject in need or at risk thereof, by administering to the subject a composition comprising a mixture of phages that can infect and lyse one or more strains of P. acnes bacteria in combination with one or more topical or oral agents selected from a list comprising an antibiotic, an anti-comedonal, an anti-P. acnes agent other than a bacteriophage, an anti-inflammatory, an anti-seborrhoeic agent, a keratolytic agent, a sebum penetration enhancer, and a sunscreen. In some embodiments, the application provides a method for reducing the amount of P. acnes in/on the skin, eyes, and/or teeth of a subject, by administering to the subject a composition comprising a mixture of bacteriophages that are capable of lysing one or more strains of P. acnes bacteria.

In some embodiments, the antibiotic agent is an antibiotic gel, an antibiotic cream, an antibiotic lotion or an oral antibiotic. In some embodiments, the anti-comedonal agent comprises one or more of a retinoid, azelaic acid and isotretinoin. In some embodiments, the anti-P. acnes agent comprises one or more of benzoyl peroxide, dapsone, azelaic acid, erythromycin, tetracycline and clindamycin, sodium sulfacetamide, adapalene, minocycline, trimethoprim, nadifloxacin, ofloxacin, doxycycline, ampicillin, cephalexin, gentamycin, and trimethoprimsulfamethoxazole. In some embodiments, the anti-inflammatory agent comprises one or more of tetracycline, erythromycin, clindamycin, nicotinamide, minocycline, trimethoprim and isotretinoin. In some embodiments, the anti-seborrhoeic agent comprises one or more of spironolactone, Dianette™ (cyproterone acetate and ethinylestradiol) and isotretinoin.

In some embodiments, the keratolytic agent comprises one or more of glycolic acid, lactic acid, mandelic acid, hydroxycapric acid, phytic acid, malic acid, citric acid, tartaric acid, salicylic acid, urea, and sulfur. In some embodiments, the sebum penetration enhancer comprises one or more of a sebum softener, sebum solubilizer and an emulsifier. In some embodiments, the sebum penetration enhancer comprises one or more of polysorbates or other non-ionic surfactants (e.g. polysorbates 20, 80 etc.), unsaturated fatty acids (e.g. oleic acid), unsaturated alcohols (e.g. oleyl alcohol), aliphatic alcohols (e.g. ethanol and isopropyl alcohol), transcutol (diethylene glycol monoethyl ether), phospholipids, unsaturated triglycerides, propylene glycol, and dipropylene glycol.

In some embodiments, the bacteriophage composition is administered every 12 hours, 24 hours, 48 hours or 72 hours at a dose of 10⁵ to 10¹³ plaque forming units (PFU) for the composition as a whole. In preferred embodiments, the bacteriophage composition is administered every 12 hours, 24 hours, 48 hours, or 72 hours at a dose of 10⁷ to 10⁹ PFU. In other preferred embodiments, the bacteriophage composition is administered every 12 hours, 24 hours, 48 hours, or 72 hours at a dose of 10⁶ to 10¹¹ PFU. In yet other preferred embodiments, the bacteriophage composition is administered every 12 hours, 24 hours, 48 hours, or 72 hours at a dose of 10⁷ to 10⁹ PFU.

The different bacteriophage present in the compositions disclosed herein may be present in equal or non-equal titer amounts. In some embodiments, the specified bacteriophage are present at equal titers (e.g., 1:1, first bacteriophage:second bacteriophage; 1:1:1, first bacteriophage:second bacteriophage:third bacteriophage, etc.). In other embodiments phage may be present at different relative titers. For example, a higher titer ratio of a specific bacteriophage to the other bacteriophage may be useful when the former bacteriophage has lower stability over time in the final formulation than the latter bacteriophage(s). In such a case the amount of the bacteriophage of lower stability may be increased so as to maintain a minimum titer over a desired product shelf life. In such embodiments, the ratio of less stable bacteriophage to more stable bacteriophages can be, e.g., 2:1, 5:1, 10:1, 25:1, 50:1, 100:1, 500:1, 1,000:1, 5,000:1 or 10,000:1 or the range of any ratios between 2:1 to 10,000:1.

In still other embodiments, a low manufacturing yield of one of the bacteriophage and/or volume limitations during formulation may lead to such bacteriophage being present at a lower titer compared to the other bacteriophage(s) in the composition. In these embodiments, the former bacteriophage may be present at up to 10,000 times lower initial titer than the other bacteriophage(s) in the composition. In such embodiments, the titer ratio of the former bacteriophage to the other bacteriophage(s) can be, e.g., 1:2, 1:5, 1:10, 1:25, 1:50, 1:100, 1:500, 1:1,000, 1:5,000 or 1:10,000.

In some embodiments, the acne is acne vulgaris, that is presented as lesions. In some embodiments, the lesions are non-inflamed or inflamed. In some embodiments, the non-inflamed lesions are comedones. In some embodiments, the inflamed lesions are papules, pustules, nodules or cysts. In some embodiments, the comedones are blackheads or whiteheads. In some embodiments, the acne is acne conglobata, acne fulminans, Hidradentis suppurativa, scalp acne (scalp folliculitis, acne necrotica miliaris or Propionibacterium folliculitis), acne associated with Progressive Macular Hypomelanosis, acne associated with SAPHO syndrome, or acne associated with Fatal Bacterial Granuloma after Trauma.

In some embodiments, the subject presents with a skin disease associated with one or more strains of P. acnes bacteria, or is at risk of developing such disease.

In some aspects, the application provides a method of selecting a mixture of phages that can treat a subject, comprising the steps of (i) obtaining a biological sample from the affected area or area to be treated or potentially treated of the subject, (ii) culturing bacteria obtained from the biological sample, (iii) inoculating the cultured bacteria with a mixture of bacteriophages, and (iv) determining which if any of the cultured bacteria are lysed by the mixture of bacteriophages, wherein when any of the cultured bacteria are lysed by the mixture of bacteriophages, the subject is determined to be treatable by the mixture of bacteriophages.

In some aspects, the application provides a composition comprising a mixture of phages that are capable of lysing one or more strains of P. acnes bacteria, formulated for pretreatment of an implant.

In some embodiments, the application provides a method for treating or preventing the development of a P. acnes containing biofilm on an implant, or reducing the amount of P. acnes in a biofilm on an implant, the method comprising applying to the implant, a composition comprising a mixture of bacteriophages that are capable of lysing one or more strains of a P. acnes bacterium. The following one or more may also be applied to the implant simultaneously or sequentially with the bacteriophage composition, i.e. from a single formulation or from separate formulations packaged either together or individually: an antimicrobial substance, a hydrophilic polymer coating, a polymer brush coating, or a contact-killing surface coating. In some embodiments, the implant releases the phage and, optionally, an antimicrobial substance from a coating or is impregnated with an antimicrobial substance. In some embodiments, the coating is one or more of a hydrogel, a nanotube, a microporous calcium phosphate coating, a mesh coating, a nanoparticle coating, a microsphere coating or a polymer coating. In some embodiments, the implant is manufactured using 3D-printing or electrospinning and incorporates the antimicrobial substance within the implant.

In some embodiments, the application provides a method for treating or preventing the development of P. acnes biofilm on an implant, the method comprising applying on an implant, a composition comprising a mixture of bacteriophages that are capable of lysing one or more strains of a P. acnes bacterium, formulated for pretreatment of an implant. In some aspects of these embodiments, the implant is a sensory or neurological implant, a cardiovascular medical device, an orthopedic implant or biomaterial, a contraceptive implant, a cosmetic implant, a dental implant, a prosthetic device, an orthopedic biomaterial, or an implant for organ dysfunction.

In some embodiments, the sensory or neurological implant is an intraocular lens, contact lens, scleral buckle, conjunctival plug, lacrimal intubation device, orbital implant, suture material, intrastromal corneal ring segment, cochlear implant, tympanostomy tube, or a neurostimulator. In some embodiments, the sensory or neurological implant is used in one or more conditions selected from a list comprising cataract, glaucoma, keratoconus, visual impairments, otosclerosis, hearing loss impairments, otitis media, middle ear diseases, epilepsy, Parkinson's disease, and treatment-resistant depression.

In some embodiments, the cardiovascular medical device is an artificial heart, artificial heart valve, implantable cardioverter-defibrillator, cardiac pacemaker, or a coronary stent. In some embodiments, the cardiovascular medical device is used in one or more conditions selected from a list comprising heart failure, cardiac arrhythmia, ventricular tachycardia, valvular heart disease, angina pectoris, and atherosclerosis.

In some embodiments, the orthopedic implant or biomaterial is a pin, rod, screw, plate, metallic glass, or a biodegradable medical implant. In some embodiments, the orthopedic implant or biomaterial is used in one or more conditions selected from a list comprising bone fractures, osteoarthritis, scoliosis, spinal stenosis, and chronic pain.

In some embodiments, the contraceptive implant is a copper- or hormone-based intrauterine device. In some embodiments, the contraceptive implant is used in one or more conditions selected from a list comprising preventing an unintended pregnancy, menorrhagia and polycystic ovarian syndrome.

In some embodiments, the cosmetic implant is a prosthetic, breast implant, shoulder implant, nose prosthesis, or ocular prosthesis. In some embodiments, the cosmetic implant is used in one or more conditions selected from a list comprising mastectomy, augmentation of the buttock, and augmentation of the chin.

In some embodiments, the implant for organ dysfunction is the LINX, implantable gastric stimulator, diaphragmatic/phrenic nerve stimulator, neurostimulator, surgical mesh, or penile prosthesis. In some embodiments, the implant for organ dysfunction is used in one or more conditions selected from a list comprising gastroesophageal reflux disease, gastroparesis, respiratory failure, sleep apnea, urinary and fecal incontinence, and erectile dysfunction.

In some embodiments of any of the above methods, any one or more of the phage set forth in Table 1 is present in the composition and comprises the nucleotide sequence of the SEQ ID NO for that phage as indicated in Table 1.

TABLE 1
Nucleotide sequence identification numbers
of the phages used in this study.

| SEQ | | Size |
|---|---|---|
| ID | | (base |
| NO. | Brief Description | pairs) |
| 1 | NS13 Bacteriophage sequence | 29,671 |
| 2 | NS19-1 Bacteriophage sequence | 30,056 |
| 3 | NS7-1 Bacteriophage sequence | 29,695 |
| 4 | PA1-11 Bacteriophage sequence | 29,491 |
| 5 | PA1-12 Bacteriophage sequence | 29,197 |
| 6 | PA1-13 Bacteriophage sequence | 29,968 |
| 7 | PA1-14 Bacteriophage sequence | 29,780 |
| 8 | PA1-4 Bacteriophage sequence | 29,097 |
| 9 | PA1-9 Bacteriophage sequence | 29,335 |
| 10 | PA2-13 Bacteriophage sequence | 29,698 |
| 11 | PA2-4 Bacteriophage sequence | 29,637 |
| 12 | PA2-7 Bacteriophage sequence | 29,724 |
| 13 | PAP-1 Bacteriophage sequence | 29,581 |
| 14 | PAP-11 Bacteriophage sequence | 29,912 |
| 15 | PAP-12 Bacteriophage sequence | 29,568 |
| 16 | PAP-13 Bacteriophage sequence | 29,960 |
| 17 | PAP-14 Bacteriophage sequence | 30,343 |
| 18 | PAP-4 Bacteriophage sequence | 29,835 |
| 19 | PAP-7 Bacteriophage sequence | 29,917 |
| 20 | PAP-8 Bacteriophage sequence | 29,385 |
| 21 | PS7-1 Bacteriophage sequence | 29,882 |

### BRIEF DESCRIPTION OF THE DRAWINGS

The foregoing summary, as well as the following detailed description, may be better understood when read in conjunction with the appended drawings. For the purpose of illustration, there are shown in the drawings embodiment(s) which are presently preferred. It should be understood, however, that the application is not limited to the precise arrangements and instrumentalities shown.

FIG. 1. Host range of the phage on P. acnes strains. Host range analysis for phage isolated on the P. acnes strains PA1, PA2 and PAP is performed on nine additional P. acnes strains; PA3, PA4, PA5, PA6, PA7, PA8, PA9, PA10 and PA11. Each of the phage are added (10 μL) to bacterial lawns of the different P. acnes strains, in 48 well plates by drop assay. Plates are incubated for overnight (37° C.) in anaerobic conditions, after which plaques become visible on the bacterial lawns. Plates with bacterial lawns of PA1, PA2 and PAP and their respective phage served as positive control. Host range is tested for each of the phage using 10 μL containing 10⁴ phage per well. +++—plaques too numerous to count or total clearing, ++—countable number of plaques greater than 10, +—1 to 10 visible plaques, −—no visible plaques.

FIG. 2. Host range of phage on a subset of the clinical isolates of P. acnes strains. The phage sensitivity of P. acnes strains isolated from skin samples of healthy volunteers or acne patients is tested. Host range analysis for phage isolated on the P. acnes strains PA1, PA2 and PAP is performed on 14 strains obtained from clinical isolates of P. acnes. Each of the phage are added (10 μL) to bacterial lawns of the different P. acnes strains, in 48 well plates by drop assay. Plates are incubated for 24 hrs (37° C.) in anaerobic conditions, after which plaques become visible on the bacterial lawns. Host range is tested for each of the phage using either the drop assay as above (10 μL containing 10⁴ phage) or a modified drop assay using instead 5 μL containing 10⁴ phage per well. +++—plaques too numerous to count or total clearing, ++—countable number of plaques greater than 10, +—1 to 10 visible plaques, −—no visible plaques, NT—not tested.

FIG. 3. Non-infectivity of P. granulosum clinical strain PAC4 by P. acnes phage. Host range analysis for phage isolated on the P. acnes strains PA1, PA2 and PAP is performed on a P. granulosum strain PAC4. Each of the phage are added (10 μL) to bacterial lawns of the P. granulosum strain in 48 well plates by drop assay. Plates are incubated overnight (37° C.) in anaerobic conditions, after which plaques become visible on the bacterial lawns. Host range is tested for each of the phage at a concentration of 10⁶ PFU/mL. +++—plaques too numerous to count or total clearing, ++—countable number of plaques greater than 10, +—1 to 10 visible plaques, −—no visible plaques.

FIG. 4. Susceptibility of B9 mutant bacteria to phage to which original PA3 strain is sensitive. The ability of phage to infect the B9 mutant of the P. acnes PA3 strain which was isolated based on the resistance it had developed to PAP-1 was examined as described. Each of the phage are added (10 μL) to bacterial lawns of the B9 strain, in 48 well plates by drop assay. Plates are incubated overnight (37° C.) in anaerobic conditions, after which plaques become visible on the bacterial lawns. The ability of each phage to infect the B9 mutant strain is tested at a concentration of 10⁶ PFU/mL. R-resistant to the infection with the tested phage, S-sensitive to the infection with the tested phage.

FIG. 5. Non-interference of other phage with PAP-12 infection of host PA4. The ability to combine several phage in a mixture without impairing the specific function of any individual phage is tested as described. The phage sensitivity of the PA4 strain (grown in BHIS) is tested on the phage isolated on the P. acnes strains PA1, PA2 and PAP in the presence or absence of the phage PAP-12. Each of the phage, mixed either in a 1:1 ratio with fresh BHIS or 1:1 with the stock of PAP-12 are added (10 μL) to bacterial lawns of the PA4 strain in 48 well plates by drop assay. Plates are incubated overnight (37° C.) in anaerobic conditions, after which plaques become visible on the bacterial lawns. Each of the phage is tested at a concentration of 10⁶ PFU/mL. +++—plaques too numerous to count or total clearing, ++—countable number of plaques greater than 10, +—1 to 10 visible plaques, −—no visible plaques.

FIG. 6. Phage versus antibiotic sensitivity of a subset of clinical strains. The susceptibility of various clinical P. acnes and P. granulosum strains to individual phage, phage mixtures and antibiotic application is tested as described. Each of the phage or phage mixtures are either tested using the drop assay as described before, or added (5 μL) to bacterial lawns of the different bacterial strains. Plates are incubated for 24 hrs (37° C.) in anaerobic conditions, after which plaques become visible on the bacterial lawns. Host susceptibility is tested for each of the phage using 5 μL containing 10⁴ phage. Antibiotic susceptibility of the strains is measured by placing 5-10 μL of the antibiotic on a bacterial lawn of each strain, and incubating overnight at 37° C. in anaerobic conditions. ++—total clearing, +—partial clearing, −—no clearing, NT—not tested; Cocktail 1-PA1-13+PS7-1+PA1-9; Cocktail 2-PA1-13+PS7-1+PAP-12.

FIG. 7. Prevention of car swelling by phage in mouse model of P. acnes infection. 3 groups of mice are subjected to model induction by a single intradermal injection of 20 μL P. acnes strain PA1 (10¹⁰ CFU/mL) in the right ear along with 20 μL of PBS in the left ear as control. (group 1; n=20). In the treatment groups, 20 μL of phage suspension (PAP-7) is injected into the right ear and 20 μL PBS into the left ear 3 hours post PA1 injection (group 2; n=20), or 20 μL PAP-7 into the right ear and 20 μL PBS into the left ear 3 hours before PA1 injection (group 3; n=20). Group 1 serves as a control for model induction. The ratio of right/left ear thickness measurements is determined in mice immediately before model induction, and 24 hrs, 48 hrs and 72 hrs post model induction. Error bars indicate mean±SEM. p=0.03, group 1 vs group 3 at 24 hrs.

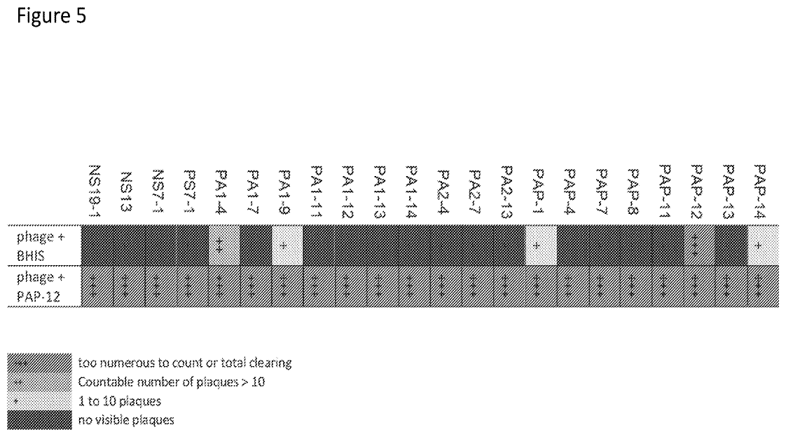

FIG. 5. Phage activity demonstrated in topical mouse model of swelling caused by P. acnes. 3 groups of mice are subjected to model induction by gentle scratching of the ear, followed by a single topical application of 10 μL PA1 suspension (10¹⁰ CFU/mL) (Groups 2, 3, and 4) or PBS (Group 1), subsequently followed by phage administration 30 min and 3 hrs after model induction as outlined in FIG. 8. Group 2 receives 10 μL of phage suspension (PS7-1, PAP-1, PAP-12) containing 10⁸ CFU/mL while Group 3 received 10 μL of phage suspension containing 10¹¹ CFU/mL. Ear thickness is measured 24 hrs after model induction. Error bars indicate mean±SEM. P=0.03 (group 3); P=0.02 (group 4).

FIG. 9. Increased time to appearance of PA3 resistant mutant demonstrated in the presence of a phage mixture vs a single phage. FIG. 9 illustrates the growth characteristics of the PA3 strain cultured alone or in the presence of phage PAP-1, PA1-4, or a combination of PAP-1 and PA1-4. PA3 is cultured overnight anaerobically at 37° C. in a 4 mL BHIS culture tube, diluted the morning after 1:3, incubated for 4 hours until reaching OD 0.8-1.2 and further diluted to OD 0.2. Phages PAP-1 and PA1-4 are diluted to 10⁸ PFU/mL, and a 1:1 mixture of these phage at this concentration is also prepared. The PA3 culture is dispensed at 190 μL per well into a 96-well plate, after which 10 μL of the phage containing samples are added in triplicates. Finally, this is covered with 40 μL of mineral oil. The plate is sealed and incubated in a Tecan M200 Pro plate reader at 37° C. to follow the infection dynamics with measurements every 15 minutes.

FIG. 10. Genetic analysis of phage. Percent homology of phages isolated against PA1, PA2, and PAP. Percent homology between the phages genomes is determined by combining all non-overlapping BLASTN alignment segments (BLAST HSPs), summing the values of their “Number of identical matches” and dividing this sum by the length of the longer of the two sequences. In some embodiments, this results in a non-symmetrical matrix.

FIG. 11. Efficacy of phage cocktail on clinical P. acnes strains. FIG. 11(a-c) show the therapeutic efficacy of the phage cocktail (PS7-1, PA1-13, and PAP-12) on 119 clinical P. acnes strains isolated from 50 volunteers, 26 of whom had acne vulgaris grades 1-4 was examined as described in Example 12. R-resistant to the infection with the tested phage cocktail, S-sensitive to the infection with the tested phage cocktail.

FIG. 12. Efficacy of phage cocktail on clinical P. acnes strains. The therapeutic efficacy of the phage cocktail (PS7-1, PA1-13, and PAP-12) on 24 antibiotic resistant clinical P. acnes strains isolated from 10 volunteers, 6 of whom had acne vulgaris grades 1-4 was examined as described in Example 12. The susceptibility of these strains to various antibiotics was also measured as described in Example 12. R-resistant to the infection with the tested phage cocktail, S-sensitive to the infection with the tested phage cocktail, C-resistant to 0.5 μg/ml clindamycin, E-resistant to 0.5 μg/ml erythromycin, T-resistant to 5 μg/ml tetracycline, M-resistant to 5 μg/ml minocycline.

### DETAILED DESCRIPTION

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present application will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N Y (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N Y (1998); Coligan et al., Short Protocols in Protein Science, John Wiley & Sons, N Y (2003); Short Protocols in Molecular Biology (Wiley and Sons, 1999).

The nomenclatures used in connection with, and the laboratory procedures and techniques of biochemistry, immunology, microbiology, molecular biology, and virology described herein are those well-known and commonly used in the art.

Throughout this specification and embodiments, the word “comprise,” or variations such as “comprises” or “comprising,” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range. As used herein, the term “about” permits a variation of ±10% within the range of the significant digit.

Notwithstanding that the disclosed numerical ranges and parameters are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments are described in terms of a Markush group or other grouping of alternatives, the present application encompasses not only the entire group listed as a whole, but each member of the group individually and all possible subgroups of the main group, and also the main group absent one or more of the group members. The present application also envisages the explicit exclusion of one or more of any of the group members in the Markush group or other grouping of alternatives.

Exemplary methods and materials are described herein, although methods and materials similar or equivalent to those described herein can also be used in the practice or testing of the various aspects and embodiments. The materials, methods, and examples are illustrative only and not intended to be limiting.

### Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “treat” and its cognates refers to a full or partial amelioration or modulation of acne or at least one discernible symptom thereof. In some embodiments, “treat” and “modulate” and their cognates refer to an amelioration of at least one measurable physical parameter, not necessarily discernible by the patient. In some embodiments, “treat” and its cognates refers to inhibiting or reducing or slowing the progression of acne, either physically (e.g., stabilization of a discernible symptom), physiologically (e.g., stabilization of a physical parameter), or both, relative to an untreated control. In certain embodiments, “treat” and its cognates refers to slowing the progression or reversing the progression of acne relative to an untreated control.

As used herein, “prevent” and its cognates refer to delaying the onset or delaying the time of return of or reducing the risk of acquiring acne or a symptom associated with acne relative to an untreated control, or delaying the onset or reducing the risk of developing a biofilm comprising one or more P. acnes strains in/on skin, eyes, teeth or an implant, or delaying the onset or reducing the risk of acquiring an implant-related internal infection associated with P. acnes or a symptom associated with an implant-related internal infection associated with P. acnes relative to an untreated control. As used herein, “delaying the time of the return” and its cognates refer to delaying a recurrence of acne or a symptom associated with acne in an individual susceptible to developing acne relative to an untreated control.

As defined here, the term “infect” or “capable of infecting” refers to the ability of a phage to kill and lyse the host cell. The ability of a phage to infect P. acnes is determined in a spot drop assay or in solution as described herein wherein the formation of plaques or a clearing zone on a soft agar plate with P. acnes colonies or reduction in optical density indicating bacterial concentration demonstrates that the phage had successful infection. As defined herein, the term “lyse” or “lysis” refers to the ability of a phage to cause dissolution or destruction of P. acnes cell membranes, thereby eliminating the P. acnes. A bacteriophage present in the compositions of this invention is considered capable of “lysing” or “infecting and lysing” a bacterial strain when the bacterial strain has a sensitivity to the phage of S (for B9 strains), or ++ or +++ for any other strain.

As defined herein, the term “lytic specificity” refers to the ability of a phage to lyse a particular designated P. acnes strain. Lytic specificity is determined by the ability of a phage, when used at a concentration of about 10⁶ PFU/mL in a solid media assay, to infect and lyse P. acnes colonies seen as a lawn on a soft agar plate at a +, ++, or +++ level, where, ++ indicates a countable number of plaques greater than 10, and +++ indicates that the plaques are too numerous to count or total clearing. In most preferred embodiments, the lytic specificity of the phage for a particular strain of P. acnes is at a +++ level. In other preferred embodiments, the lytic specificity of the phage for a particular strain of P. acnes is at a ++ level. In some embodiments, the lytic specificity of a phage for the B9 strain is determined by the ability of a phage to infect and lyse B9colonies on a lawn on a soft agar plate at an R, or S level where R indicates that the B9 strains is resistant to the infection with the tested phage, and S indicates that the B9 strain is sensitive to the infection with the tested phage. For the purposes of this application, an “S” designation for infection of B9 means the appearance of any plaques in at least two individual experiments wherein the tested phage is used to infect and lyse B9 colonies on a soft agar plate.

As used herein, the term “acne” refers to a skin condition that occurs when hair follicles become plugged with oil and dead skin cells. It is characterized by whiteheads, blackheads pimples, nodules, cysts, oily skin and scarring that can appear on various body structures including but not limited to the face, forehead, chest, upper back and shoulders. The major pathophysiological features of acne include but are not limited to hyperkeratinization, sebum production, bacterial proliferation and inflammation. In some embodiments, acne is associated with inflammatory activity of one or more strains of P. acnes. In some embodiments, acne is characterized by noninflammatory, open or closed comedones and by inflammatory papules, pustules, and nodules. As used herein, acne refers to acne vulgaris, acne conglobata, acne fulminans, Hidradentis suppurativa, scalp acne, acne associated with Progressive Macular Hypomelanosis, acne associated with SAPHO syndrome or acne associated with Fatal Bacterial Granuloma after Trauma.

As used herein, the term “biofilm” refers to a sessile community of microbial cells, comprising one or more P. acnes strains, that (i) are attached to a substratum, interface, or each other, (ii) are embedded in a matrix of (at least partially self-produced) extracellular polymeric substances; and (iii) exhibit an altered phenotype with regard to growth, gene expression, and protein production compared to planktonic bacterial cells (Achermann et al., 2014). The basic ingredients of a biofilm are microbes, glycocalyx, and surface (Dunne WM, 2002). The biofilm matrix may be composed of endogenously and exogenously produced polysaccharides, protein, and/or extracellular DNA, in proportions based on the biofilm growth environment and the bacterial genera, species, and strains involved (Archer et al., 2011). The organized biofilm communities, which can range from a single cell to a thick multicellular layer, have structural and functional heterogeneity (Costerton et al., 1999). The different structures are dependent on localized environmental conditions such as nutrition, waste, gas, and space limitations (Dunne WM, 2002). In certain embodiments, the biofilm is a single-species biofilm. In other embodiments, the biofilm is polymicrobial and comprising one or more strains of P. acnes bacteria and one or more strains of other non-P. acnes bacteria.

In certain embodiments, the one or more bacteriophage described herein is administered to treat acne in a subject and results in one or more symptoms or physical parameters of the condition or disorder to improve by at least about 10%, 20%, 30%, 40%, 50%, 60%, 70%, 80%, 90%, 95%, or more as compared to levels in an untreated or control subject. In some embodiments, the improvement is measured by comparing the symptom or physical parameter in a subject before and after administration of the bacteriophage. In some embodiments, the physical parameters tested include but are not limited to reduction in the number, percentage, and/or severity of non-inflammatory lesions, and reduction in the number, percentage, and/or severity of inflammatory lesions. In some embodiments, the physical parameters are assessed qualitatively by a certified clinician.

In some embodiments, the one or more bacteriophage described herein is administered to prevent or retard the development of biofilm comprising one or more P. acnes strains, in/on a subject's skin, a subject's eyes, teeth, or an implant inserted in a subject, and results in the one or more physical parameters on the skin, eyes, teeth, or implant to improve by at least about 10%, 20%, 30%, 40%, 50%, 60%, 70%, 80%, 90%, 95%, or more as compared to levels in an untreated or control subject. In some embodiments, the improvement is measured by comparing the physical parameter in a subject before and after administration of the bacteriophage. In some embodiments, the implant is inserted in a subject after it is pretreated with the one of more bacteriophage described herein.

The measurable physical parameter may be any suitable parameters known in the art, e.g., for biofilm formation and development (Tyner and Patel, 2016) or for implant-related infection. Biofilm formation and development parameters include but are not limited to assessment of microbial attachment, maturation and dispersion, and biofilm thickness. The biofilm formed in/on skin, teeth or an implant can be analyzed by scanning electron microscopy (EM) and by transmission EM (Holmberg et al., 2009) fluorescent in situ hybridization, immunofluorescent microscopy (Brandwein et al., 2016). In addition, biofilm formation on teeth include but are not limited to assessment of dental plaque. Implant-related infection parameters include but are not limited to detection of P. acnes in intraoperative tissue cultures. Tissue samples, aspirates and biopsy samples can be obtained from a patient and cultured in both agar plate and broth in aerobic and anaerobic conditions for greater than 5 days to optimize the sensitivity and specificity to detect P. acnes (Saper et al., 2015). A patient may present with clinical symptoms of P. acnes infection including but not limited to erythema, swelling, skin reaction, exudate production, pain, stiffness, and implant loosening.

Subjects in need of prevention or treatment may include individuals already having acne, as well as those at risk of having, or who may ultimately acquire acne. The need for prevention or treatment is assessed, e.g., by the presence of one or more risk factors associated with the development of acne, or the presence or progression of acne. For example, “preventing” or “treating” acne may encompass inhibiting the onset of associated symptoms, or reducing or eliminating associated symptoms, and does not necessarily encompass the elimination of the underlying disease etiology, e.g., genetic or environmental factors.

Subjects in need of prevention or treatment may also include individuals having biofilms comprising one or more P. acnes strains in/on their skin, eyes or teeth, as well as those at risk of having, or who may ultimately acquire biofilms comprising one or more P. acnes strains. The need for prevention or treatment is assessed, e.g., by the presence of one or more risk factors associated with the development of a biofilm, the presence or progression of a biofilm, or likely receptiveness to prevention of biofilm development in a subject or on an implant, or treatment of a subject having a biofilm. For example, “preventing” or “treating” the formation of a biofilm on skin may encompass reducing or eliminating associated symptoms of acne.

In some embodiments, a subject with acne is in remission and/or presently asymptomatic, and the bacteriophage described herein may be administered during the remission period to reduce the potential for a flare-up. In some embodiments, the individual in remission and/or presently asymptomatic for acne is undergoing treatment, e.g., antibiotics, anti-P. acnes agents, anti-comedonals, anti-inflammatories, anti-seborrhoeic agents, and/or anti-P. acnes vaccine, and the bacteriophage described herein may be co-administered with such treatment to reduce the potential for a flare-up.

As used herein, “P. acnes” (also known as Bacillus acnes, Corynebacterium acnes, Cutibacterium acnes or Corynebacterium parvumis) is a microaerophilic, anaerobic-aerotolerant Gram-positive, pleiomorphic rod-shaped bacterium that resides on the surface of and in the pilosebaceous follicles of the human skin, oral cavity, conjunctiva, intestinal tract and external ear canal belonging to the Propionibacteriaceae family (Perry and Lambert, 2011). In some embodiments, P. acnes refers to naturally occurring P. acnes. In some embodiments, P. acnes refers to naturally occurring, variant or mutant P. acnes (e.g., antibiotic resistant, phage resistant, nosocomial). In some embodiments, the variant or mutant P. acnes is resistant to at least 1, at least 2, at least 3, at least 4, or at least 5 antibiotics. In some embodiments, a mutant bacterial strain may arise in the presence of a bacteriophage and become resistant to said bacteriophage.

As used herein, a “strain” of bacteria refers to a genetic variant or subtype of bacteria. In some embodiments, a “strain” of bacteria comprises descendants from a single isolation in a pure culture of said bacteria. As used herein, a “strain” of bacteria may refer to one or more genetic variants or subtypes of said bacteria. For example, as used herein, a “strain” of P. acnes may refer to one or more genetic variants or subtypes of P. acnes, including but not limited to PA1 (ATCC 11828), PA2 (ATCC 33179), PA3 (ATCC 29399) PA4 (DSM 32714, deposited on Dec. 5, 2017), PA5 (ATCC 51277), PA6 (ATCC 6923), PA7 (ATCC 6922), PA8 (ATCC 6921), PA9 (ATCC 12930), PA10 (ATCC 6919), PA11 (ATCC 11827), PA13 (DSM 16379), PAP (DSM 32709, deposited on Dec. 5, 2017), B9 (DSM 32711, deposited on Dec. 5, 2017), PAC1, PAC2, PAC3, PAC5, PAC6, PA12, PAC13, PAC14, 2001-1, 2001-3, 2001-5, 2002-3, 2002-7, 2002-9, 2003-2, 2003-10, 2004-8_2, 2002-8_3, 2002-8_4, 2002-8_5, 2002-8_6, and 2002-8_10. Similarly, as used herein, a bacteriophage that is capable of lysing a “strain” of P. acnes refers to a bacteriophage that is capable of lysing one or more genetic variants or subtypes of P. acnes, including but not limited to PA1, PA2, PA3, PA4, PA5, PA6, PA7, PA8, PA9, PA10, PA11, PA13, PAP, B9, PAC1, PAC2, PAC3, PAC5, PAC6, PAC7, PAC8, PAC9, PAC10, PAC11, PAC12, PAC13, PAC14, 2001-1, 2001-3, 2001-5, 2002-3, 2002-7, 2002-9, 2003-2, 2003-10, 2004-8_2, 2002-8_3, 2002-8_4, 2002-8_5, 2002-8_6, and 2002-8_10.

In some embodiments, as used herein, a “mutant” bacterium refers to a bacterium that comprises greater than about 85%, greater than about 90%, greater than about 95%, greater than about 97%, or greater than about 99% homology to a corresponding wild-type bacterial strain.

As used herein, “bacteriophage” and “phage” are used interchangeably and refer to an isolated virus that is capable of infecting a bacterium. In some embodiments, the phage comprises a DNA or an RNA genome. A phage may be isolated from a natural or human-made environment. In some embodiments, the phage is selected from Siphoviridae. In some embodiments, the phage has a GC content of about 53% to about 55% (Marinelli L J 2012). In some embodiments, the genomic percentage identity between all P. acnes phages ranges from about 80% to 100%. As used herein, a “P. acnes bacteriophage” is intended to refer to a bacteriophage that is capable of lysing a P. acnes bacterium. For example, a “bacteriophage” used herein may refer to one or more genetic variants or subtypes of P. acnes bacteriophage including but not limited to PS7-1 (DSM xxxxx, deposited on Nov. 29, 2018), PAP-12 (DSM xxxxx, deposited on Nov. 29, 2018) PA1-13 (DSM xxxxx, deposited on Nov. 29, 2018), PA1-11 (DSM xxxxx, deposited on Nov. 29, 2018), PA1-12 (DSM xxxxx, deposited on Nov. 29, 2018), PA1-14 (DSM xxxxx, deposited on Nov. 29, 2018), PA2-4 (DSM xxxxx, deposited on Nov. 29, 2018), PA2-7 (DSM xxxxx, deposited on Nov. 29, 2018), PAP-1 (DSM xxxxx, deposited on Nov. 29, 2018, PAP-4 (DSM xxxxx, deposited on Nov. 29, 2018), PAP-Il (DSM xxxxx, deposited on Nov. 29, 2018), PAP-13 (DSM xxxxx, deposited on Nov. 29, 2018), PAP-14 (DSM xxxxx, deposited on Nov. 29, 2018), NS13 (DSM xxxxx, deposited on Nov. 29, 2018), NS7-1 (DSM xxxxx, deposited on Nov. 29, 2018), PA1-9 (DSM xxxxx, deposited on Nov. 29, 2018), PAP-7 (DSM xxxxx, deposited on Nov. 29, 2018), PAP-8 (DSM xxxxx, deposited on Nov. 29, 2018), NS19-1 (DSM xxxxx, deposited on Dec. 4, 2018), PA1-4 (DSM xxxxx, deposited on Dec. 4, 2018), PA2-13 (DSM xxxxx, deposited on Dec. 4, 2018).

It is known that different isolates of a given bacteriophage may vary at the nucleic acid sequence level. In some embodiments, bacteriophages are considered to be “functionally equivalent” as long as they exhibit identical lytic specificity. As used herein, the term “bacteriophage” encompasses a parent bacteriophage as well as its progeny or derivatives.

As used herein, “host range” refers to the bacteria that are susceptible to infection by a particular phage. The host range of a phage may include, but is not limited to, a strain, or a species. The term encompasses phage adsorbable, productive infections. In some embodiments, a phage may recognize two or more strains. In some embodiments, a phage may recognize wild-type and phage-resistant mutant strains.

Different phage isolates may be prepared and phenotyped using methods known in the art, e.g., a solid media assay, or liquid media assay. In some embodiments, the solid media assays to quantify and isolate phage are based on plaque assays (Abedon & Yin, 2009), ranging from efficiency of plating (EOP) (Kutter, 2009) to spot testing (Hyman & Abedon, 2010). In some embodiments, the plate format used for the plaque assay can be modified, e.g., from a petri dish to a 48-well plate.

In some embodiments, a double-layer plaque assay is used to phenotype bacteriophage isolates. For example, a starter culture of 4 mL BHIS may be inoculated with 5-10 colonics from a plate. This culture may be incubated overnight at 37° C. under anaerobic conditions. A volume of 100 μL of this culture may be mixed with 100 μL of a phage-containing sample (or medium only control) and incubated for 15 minutes. Thereafter, 3 mL of BHIS top agar (pre-molten 0.4% agar BHIS supplemented with 1 mM Ca²⁺ and Mg²⁺ ions) may be added, and the mixture may be poured over a BHIS bottom agar plate (1.5% agar BHIS). The plates may be allowed to gel at room temperature, and then incubated overnight at 37° C. under anaerobic conditions until plaques are identified.

In some embodiments, a modified spot drop assay is used to phenotype bacteriophage isolates. For example, a starter culture of 4 mL BHIS may be inoculated with 5-10 colonies from a plate. This culture may be incubated at 37° C., anaerobically. At this stage, 10 μL of samples containing phage or media only controls may be dropped in the middle of the well, left to absorb, and then may be incubated overnight (37° C., anaerobically) until plaques are visible for counting.

In some embodiments, a liquid media assay is used to phenotype the bacteriophage. In some embodiments, liquid-based phage infection assays follow the time-course of infection and can provide more than quantitative end-points of infection as compared to the solid-phase plaque assays. In some embodiments, by mixing phage with bacteria in liquid medium, then following the turbidity of the culture over time, one can discern finer differences (e.g., a delay in the time of cell lysis) between how different bacterial strains interact with the phage. In some embodiments, the liquid media assay allows for high-throughput measurements by using 96-well plates and reading optical density in a plate reader.

For example, a bacterial strain may be grown for overnight, then diluted 1:10 and further grown until an OD₆₀₀ of about 0.4-0.8. This culture may then be diluted using BHIS medium to a starting optical density, typically between 0.05 and 0.2 OD₆₀₀. A volume of 200 μL of culture may then be dispensed into the wells of a Nunclon flat-bottomed 96-well plate. 10 μL of a sample containing phage or 10 μL of medium as control may be added to each well. The wells may be covered with 50 μL of mineral oil to limit evaporation, and a thin sterile optically transparent polyester film may be added to keep the culture sterile. Optical density measurements may be carried out every 15 minutes, e.g., in a Tecan Infinite M200 plate reader connected to a Tecan EVO75 robot. Between measurements, the plate may be incubated while shaking at 37° C., e.g., inside the EVO75 incubator.

In some embodiments, infectivity is determined by the plaque presence in a solid assay only. In some embodiments, infectivity is determined by the decrease in the bacterial culture optical density in a liquid assay only. In some embodiments, infectivity is determined by the decrease in the bacterial culture optical density and plaque presence in both the liquid assay and the solid assay.

As used herein, a “lytic” bacteriophage refers to a virulent bacteriophage that attaches to a bacterial host and inserts its genetic material into the bacterial host cell. After that a phage usually follows one of two life cycles, lytic (virulent) or lysogenic (temperate). Lytic phages take over the machinery of the cell to make phage components. They then destroy, or lyse, the cell, releasing new phage particles. See, e.g., Abedon et al., 2011; Sulakvelidze et al., 2001. As used herein, “lysis” refers to a detectable amount of infection.

In some embodiments, the % lysis is measured by methods known in the art and described herein, e.g., by optical density (OD).

As used herein, “% homology” refers to the level of nucleic acid sequence identity or amino acid sequence identity between a first nucleic acid or amino acid sequence when aligned to a second nucleic acid or amino acid sequence using a sequence alignment program. When a position in the first and the second sequences is occupied by the same nucleic acid or amino acid (e.g., if a position in the first nucleic acid sequence and the second nucleic acid sequence is occupied by cytosine), then the first and the second sequences are homologous at that position.

In general, homology between two sequences is calculated from the number of matching or homologous positions shared by the two sequences over the total number of positions compared. In some embodiments, the first and the second sequences are aligned in a manner to maximize % homology. In some embodiments, % homology refers to the % identity over the shorter of two sequences. In some embodiments, the % homology for a nucleic acid sequence includes intronic and/or intergenic regions. Exemplary levels of % homology include, but are not limited to, 80, 81, 82, 83, 84, 85, 86, 87, 88, 89, 90, 91, 92, 93, 94, 95, 96, 97, 98, 99% or more sequence identity between a first and a second sequence.

Exemplary sequence alignment programs that may be used to determine % homology between two sequences include, but are not limited to, the FASTA package (including rigorous (SSEARCH, LALIGN, GGSEARCH and GLSEARCH) and heuristic (FASTA, FASTX/Y, TFASTX/Y and FASTS/M/F) algorithms, the EMBOSS package (Needle, stretcher, water and matcher), the BLAST programs (including, but not limited to BLASTN, BLASTX, TBLASTX, BLASTP, TBLASTN), MEGABLAST and BLAT. In some embodiments, the sequence alignment program is BLASTN. For example, 95% homology refers to 95% sequence identity determined by BLASTN, by combining all non-overlapping alignment segments (BLAST HSPs), summing their numbers of identical matches and dividing this sum with the length of the shorter sequence.

In some embodiments, the sequence alignment program is a basic local alignment program, e.g., BLAST. In some embodiments, the sequence alignment program is a pairwise global alignment program. In some embodiments, the pairwise global alignment program is used for protein-protein alignments. In some embodiments, the pairwise global alignment program is Needle. In some embodiments, the sequence alignment program is a multiple alignment program. In some embodiments, the multiple alignment program is MAFFT. In some embodiments, the sequence alignment program is a whole genome alignment program. In some embodiments, the whole genome alignment is performed using BLASTN. In some embodiments, BLASTN is utilized without any changes to the default parameters.

As used herein, a “composition” refers to a preparation of the bacteriophage of the disclosure with other components such as a physiologically acceptable carrier and/or excipient. In some embodiments, the composition is a pharmaceutical composition. In some embodiments, the composition is a cosmetic composition.

“Physiologically acceptable carrier” is used herein to refer to a pharmaceutically or cosmetically acceptable carrier or a diluent that does not cause significant irritation to an organism and does not abrogate the biological activity and properties of the administered bacteriophage composition. An adjuvant is included under these phrases.

The term “excipient” refers to an inert substance added to a pharmaceutical composition or a cosmetic composition to further facilitate administration of an active ingredient. Examples of excipients for pharmaceutical compositions of the application include, but are not limited to, calcium bicarbonate, calcium phosphate, various sugars and types of starch, cellulose derivatives, gelatin, vegetable oils, polyethylene glycols, silicone-based excipients, DMSO, antioxidants, including, e.g., BHT, BHA, a-tocopherol, preservatives, including methylparaben and propylparaben and benzoic acid, waxes, including, e.g., cetyl esters wax, stearic acid and cetyl alcohol, humectants, including e.g., propylene glycol, glycerin, sorbitol, urea, alpha hydroxyacids, cosolvents, including e.g., glycerin, propylene glycol, and ethanol., enhancers, including e.g., water, surfactant, and propylene glycol, and surfactants, including, e.g., polysorbate 20, Triton X-100, polysorbate 80, polyoxyethylene sorbitan, fatty acid esters, different poloxamers, polyoxyl-40-stearate and other polyoxyethylene stearates, glycerol monostearate, macrogol-8-stearat, macrogol cetostearylether 20 and polyoxyethylene alkyl ethers, sorbitan monostearate and other sorbitan monoesters, polyoxyethylene castor oil derivatives, sodium lauryl sulfate, cetylpyridinium chloride, thickeners and gelling agents, including, e.g., dextran 40, dextran 70, carbomer 940, carbomer 974 and other polyacrylic acid derivates, dextrin, maltodextrin, polyvinyl alcohol, polyethylene glycol and agar. Examples of excipients for cosmetic compositions include, but are not limited to, water, oils, fats, waxes, humectants, surfactants, preservatives, perfumes and colors, herbal or plant material, functional raw material, natural surface acting agents/emulsifiers, and other cosmetically acceptable carriers or agents. See, for example, Harry's Cosmeticology, 9^{th} Ed. In some embodiments, the natural surface acting agent or emulsifier, includes, e.g., fermentation-derived glycolipids including but not limited to rhamnolipids and sophorolipids; plant derived alkyl polyglucosides including but not limited to sodium cocoyl isethionate, decyl glucoside, lauryl glucoside, coco-glucoside, and cocoamidopropyl betaine; fatty acid amides including but not limited to cocamide monoethanolamine; and phospholipids.

The terms “therapeutically effective dose” and “therapeutically effective amount” are used to refer to an amount of a compound that results in prevention, delay of onset of symptoms, or amelioration of symptoms of acne compared to an untreated control, or compared in a subject before and after administration of the bacteriophage. A therapeutically effective amount may, for example, be sufficient to treat, prevent, reduce the severity, delay the onset, and/or reduce the risk of occurrence of one or more symptoms of acne compared to an untreated control. The terms “therapeutically effective dose” and “therapeutically effective amount” are also used to refer to an amount of a compound that results in the prevention of the development of a biofilm comprising one or more P. acnes strains in/on skin, eyes, teeth, or on an implant. A therapeutically effective amount may, for example, be sufficient to prevent, reduce the severity, delay the onset, and/or reduce the risk of developing a biofilm with one or more strains of P. acnes, compared to an untreated control.

As used herein, “plaque forming units” are used to refer to the phage titer that is a quantitative measurement of the biological activity of the phage and is expressed as plaque forming units (PFU) per ml. In some embodiments, a PFU is also referred to as a unit of activity.

As used herein, “skin” refers to the organ of the integumentary system that forms the soft outer tissue covering mammals. It encompasses multiple layers of ectodermal tissue, and guards the underlying muscles, bones, ligaments and internal organs. It encompasses the epidermis, the basement membrane, the dermis and the hypodermis. The epidermis further comprises the stratum corneum, stratum lucidum (only in palms and soles), stratum granulosum, stratum spinosum, stratum germinativum (or stratum basale). The dermis further comprises the papillary region, and the reticular region. The hypodermis further comprises the deeper subcutaneous tissue made of fat and connective tissue. Bacteria can be found throughout the skin.

As used herein, “implant” refers to a medical prosthesis or cosmetic device manufactured to replace a missing biological structure, support a damaged biological structure, or enhance an existing biological structure. It encompasses sensory or neurological implants, cardiovascular medical devices, orthopedic implants or biomaterials, contraceptive implants, cosmetic implants, dental implants, prosthetic devices, or implants for organ dysfunction. “Implant” further comprises devices such as an intraocular lens, contact lens, scleral buckle, conjunctival plug, lacrimal intubation device, orbital implant, suture material, intrastromal corneal ring segment, intrastromal corneal ring segment, cochlear implant, tympanostomy tube, a neurostimulator, an artificial heart, artificial heart valve, implantable cardioverter-defibrillator, cardiac pacemaker, a coronary stent, a pin, rod, screw, plate, metallic glass, a biodegradable medical implant, a copper- or hormone-based intrauterine device, a breast implant, shoulder implant, nose prosthesis, ocular prosthesis, the LINX, implantable gastric stimulator, diaphragmatic/phrenic nerve stimulator, neurostimulator, surgical mesh, or penile prosthesis. An implant can be used in conditions including but not limited to cataract, glaucoma, keratoconus, visual impairments, otosclerosis, hearing loss impairments, otitis media, middle ear diseases, epilepsy, Parkinson's disease, treatment-resistant depression, heart failure, cardiac arrhythmia, ventricular tachycardia, valvular heart disease, angina pectoris, atherosclerosis, bone fractures, osteoarthritis, scoliosis, spinal stenosis, chronic pain, preventing an unintended pregnancy, menorrhagia, polycystic ovarian syndrome, mastectomy, augmentation of the buttock, augmentation of the chin, gastroesophageal reflux disease, gastroparesis, respiratory failure, sleep apnea, urinary and fecal incontinence, and erectile dysfunction.

“Administering” or “administration of” a substance, a compound, a composition, a formulation or an agent to a subject can be carried out using one of a variety of methods known to those skilled in the art. For example, a compound or an agent can be administered topically, by applying on the skin, teeth, eyes or on parts of eyes including but not limited to the lens capsule and the corneal stroma. For example, the composition may be in a form suitable for topical administration and be in the form of a cream, paste, solution, powder, spray, aerosol, capsule, eye drop, eye ointment, eye lotion, solid or gel, or may be bonded to a solid surface. The composition may also form part of a face wash, soap, application stick, cosmetic or dressing. Administering can also be performed, for example, once, a plurality of times, and/or over one or more extended periods. In some aspects, the administration includes both direct administration, including self-administration, and indirect administration, including the act of prescribing a formulation. For example, as used herein, a physician who instructs a patient to self-administer a formulation, or to have the formulation administered by another and/or who provides a patient with a prescription for a formulation is administering the formulation to the patient.

“Pretreatment of” an implant with a substance, a compound, a composition, a formulation or an agent can be carried out using one of a variety of methods known to those skilled in the art. For example, the implant may be coated with a formulation described herein, along with an antimicrobial substance, a hydrophilic polymer coating, a polymer brush coating, or a contact-killing surface coating, to avoid colonization of the surface by P. acnes bacteria, thereby reducing the risk of biofilm formation and clinical infections. In some embodiments, the implant is impregnated with an antimicrobial substance. In some embodiments, the formulation described herein is removed from the surface of an implant prior to insertion within the human body.

All ranges include end points. All references cited are incorporated for any purpose (specification controls where there are inconsistencies). Singular form includes plural.

Each embodiment described herein may be used individually or in combination with any other embodiment described herein.

### Bacteriophage

The bacteriophage described herein is capable of lysing one or more P. acnes bacterial strains that are generally thought to be associated with acne, e.g., acne vulgaris, acne conglobata, acne fulminans, Hidradentis suppurativa, scalp acne, acne associated with Progressive Macular Hypomelanosis, acne associated with SAPHO syndrome or acne associated with Fatal Bacterial Granuloma after Trauma. In some embodiments, the bacteriophage is capable of lysing one or more P. acnes bacterial strains that are generally thought to be associated with acne. In some embodiments, the bacteriophage is capable of lysing one or more P. acnes bacterial strains that are associated with biofilms. In some embodiments, the bacteriophage is capable of modulating acne by lysing the one or more P. acnes bacteria in a mammal. In some embodiments, the bacteriophage is capable of modulating acne by lysing the one or more P. acnes bacteria in/on the skin, eyes or teeth of a mammal. In some embodiments, the bacteriophage is capable of modulating P. acnes-associated biofilm formation by lysing the one or more P. acnes bacteria in/or the skin, the eyes, the teeth, or on the implant inserted in a mammal.

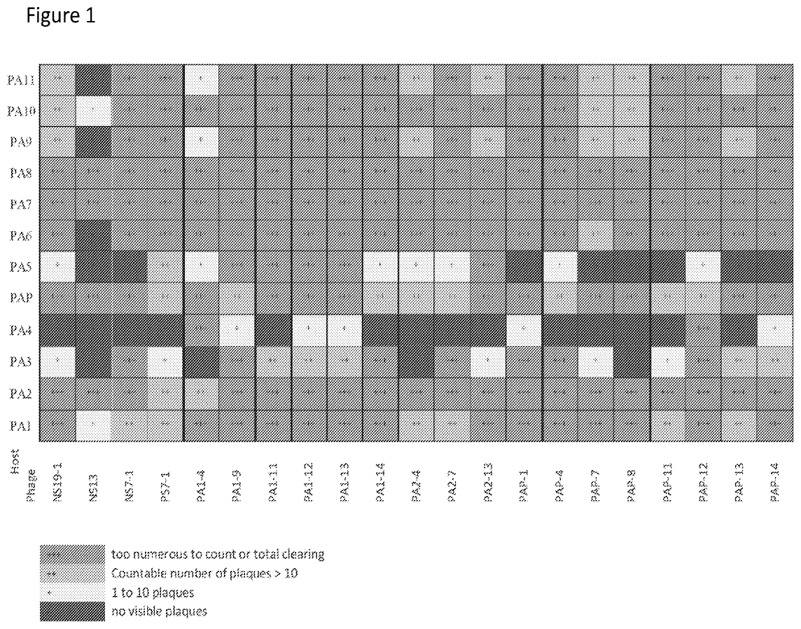

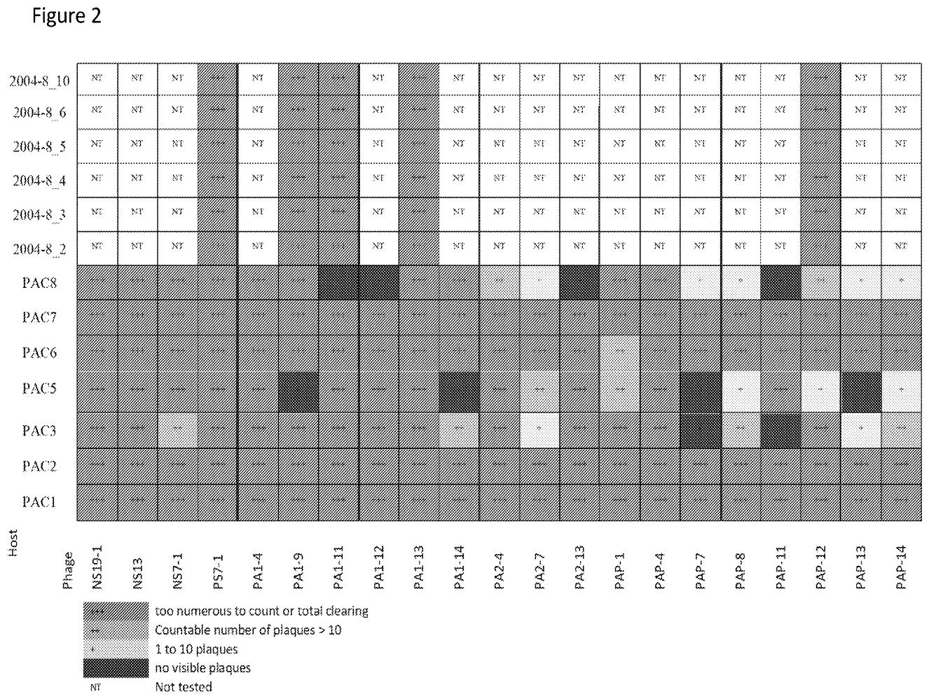

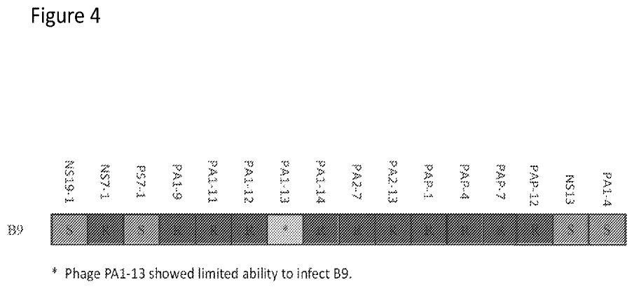

In some embodiments, the composition comprises at least one bacteriophage selected from PS7-1, NS19-1, and homologs thereof that have the same lytic specificity as PS7-1, and NS19-1. In some embodiments, the homolog comprises at least about 89%, at least about 90%, at least about 95%, at least about 97%, or at least about 99% homology to the sequence of at least one of PS7-1 and NS19-1 as measured by BLASTN. The bacteriophages identified in this paragraph are referred to as “Cluster 1a” bacteriophage and are capable of infecting P. acnes strain B9, and the bacterial strains described herein, sec, e.g., FIG. 1, FIG. 2, and FIG. 4.

In some embodiments, the composition comprises at least one bacteriophage selected from NS13, and homologs thereof that have the same lytic specificity as NS13. In some embodiments, the homolog comprises at least about 89%, at least about 90%, at least about 95%, at least about 97%, or at least about 99% homology to the sequence of NS13 as measured by BLASTN. The bacteriophages identified in this paragraph are referred to as “Cluster 1b” bacteriophage and are also capable of infecting P. acnes strain B9, and the bacterial strains described herein, but show no lytic specificity with respect to certain strains (i.e., PA1, PA3, PA4, PA5, PA6, PA9, PA10, and PA11) as compared to Cluster 1a bacteriophage, see, e.g., FIG. 1, and FIG. 2.

In some embodiments, the composition comprises at least one bacteriophage selected from PA1-13, and homologs thereof that have the same lytic specificity as PA1-13. In some embodiments, the homolog comprises at least about 89%, at least about 90%, at least about 95%, at least about 97%, or at least about 99% homology to the sequence of PA1-13 as measured by BLASTN. The bacteriophages identified in this paragraph are referred to as “Cluster 2” bacteriophage and are capable of infecting P. acnes strain PA3, and the bacterial strains described herein (i.e. PA1, PA2, PAP, PA5, PA6, PA7, PA8, PA9, PA10, and PA11), and incapable of infecting strain PA4 efficiently (i.e., at greater than + level) see, e.g., FIG. 1, and FIG. 2.

In some embodiments, the composition comprises at least one bacteriophage selected from PAP-12, and homologs thereof that have the same lytic specificity as PAP-12. In some embodiments, the homolog comprises at least about at least about 85%, at least about 87%, at least about 90%, at least about 95%, at least about 97%, or at least about 99% homology to the sequence of PAP-12 as measured by BLASTN. The bacteriophages identified in this paragraph are referred to as “Cluster 3a” bacteriophage and are capable of infecting the bacterial strains described herein (i.e. PA1, PA2, PA3, PAP, PA6, PA7, PA8, PA9, PA10, and PA11), capable of infecting strain PA4 efficiently, and incapable of infecting strain PA5 efficiently (i.e., at greater than + level) see, e.g., FIG. 1, and FIG. 2.

In some embodiments, the composition comprises at least one bacteriophage selected from PA1-12, PA1-9, PAP-14, PAP-6, PAP-1, and homologs thereof that have the same lytic specificity as PA1-12, PA1-9, PAP-14, PAP-6 and PAP-1. In some embodiments, the homolog comprises at least about 85%, at least about 87%, at least about 90%, at least about 95%, at least about 97%, or at least about 99% homology to the sequence of at least one of PA1-12, PA1-9, PAP-14, PAP-6, and PAP-1 as measured by BLASTN. The bacteriophages identified in this paragraph are referred to as “Cluster 3b” bacteriophage and are capable of infecting the bacterial strains described herein, and incapable of infecting strain PA4 efficiently (i.e., at greater than + level), see, e.g., FIG. 1, FIG. 2, and FIG. 4.

In some embodiments, the composition comprises at least one bacteriophage selected from PA2-13, PAP-8, PA1-11, PAP-13, PA2-7, PAP-11, PA1-14, PAP-7, NS7-1, PA2-4, PAP-4, and homologs thereof that have the same lytic specificity as PA2-13, PAP-8, PA1-11, PAP-13, PA2-7, PAP-11, PA1-14, PAP-7, NS7-1, PA2-4, and PAP-4. In some embodiments, the homologs comprise at least about 88%, at least about 90%, at least about 95%, at least about 97%, or at least about 99% homology to the sequence of at least one of one or more of PA2-13, PAP-8, PA1-11, PAP-13, PA2-7, PAP-11, PA1-14, PAP-7, NS7-1, PA2-4, and PAP-4 as measured by BLASTN. The bacteriophages identified in this paragraph are referred to as “Cluster 4” bacteriophage and are incapable of infecting P. acnes strains B9 and PA4, and capable of infecting the bacterial strains described herein, see, e.g., FIG. 1, FIG. 2, and FIG. 4.

In some embodiments, the composition comprises at least one bacteriophage selected from PA1-4, and homologs thereof that have the same lytic specificity as PA1-4. In some embodiments, the homologs comprise at least about 88%, at least about 90%, at least about 95%, at least about 97%, or at least about 99% homology to the sequence of at least one of one or more of PA1-4 as measured by BLASTN. The bacteriophages identified in this paragraph are referred to as “Cluster 5” bacteriophage and are capable of infecting strains B9 and PA4, as well as other bacterial strains described herein, incapable of infecting strain PA5 efficiently (i.e., at greater than + level), and incapable of infecting PA3, see, e.g., FIG. 1, FIG. 2, and FIG. 4.

In some embodiments, during phage infection, mutant bacterial strains arose that are resistant to the phage. In some embodiments, it is beneficial for a phage to target the mutant bacteria. In order to identify said phage, in some embodiments, bacteria are incubated with a phage, see, e.g., FIG. 4, in order to first generate mutant bacterial strains that can survive such infection. Different phages are then tested on the resulting mutant bacteria to identify phage that can lyse such mutant bacterial cells. Exemplary mutant B9 strain is described herein. See, e.g., FIG. 4. In some embodiments, the mutant bacterial strain mirrors mutations that are likely to occur in vivo when a subject is treated with the phage disclosed herein. In some embodiments, a bacteriophage may be generated that is capable of infecting and lysing both the original bacterial strain and the mutant bacterial strain that arose therefrom. Thus, in some embodiments, the bacteriophages provided herein are capable of treating unmodified PA3 bacteria, as well as resistant B9 mutant bacteria.

In some embodiments, the bacteriophage is capable of infecting P. acnes B9 strain and at least one bacteria, at least two bacteria, at least three bacteria, at least four bacteria, or at least five bacteria, at least six bacteria, at least seven bacteria, at least eight bacteria, at least nine bacteria, or at least ten bacteria selected from PA1, PA2, PA5, PA6, PA7, PAR, PA9, PA10, PA11, and PAP. See, e.g., FIG. 1 and FIG. 4.

In some embodiments, the bacteriophage is capable of infecting P. acnes strain PA4, and at least one bacteria, at least two bacteria, at least three bacteria, at least four bacteria, or at least five bacteria, at least six bacteria, at least seven bacteria, at least eight bacteria, at least nine bacteria, or at least ten bacteria selected from PA1, PA2, PA3, PA6, PA7, PA8, PA9, PA10, PA11, and PAP. See, e.g., FIG. 1.

In some embodiments, the bacteriophage is incapable of infecting strain B9 and strain PA4, but is capable of infecting at least one bacteria, at least two bacteria, at least three bacteria, at least four bacteria, or at least five bacteria, at least six bacteria, at least seven bacteria, or at least eight bacteria selected from PA1, PA2, PA3, PA6, PA7, PA8, PA10, and PAP. See, e.g., FIG. 1 and FIG. 4.

As used herein, a “mixture” of lytic bacteriophage refers to a composition comprising at least two different isolates of lytic bacteriophage as described herein. As used herein, “a” or “one” bacteriophage refers to an isolate or type of bacteriophage and is not necessarily intended to refer to a single bacteriophage particle.

In some embodiments, the mixture comprises a least one phage that is capable of infecting P. acnes strain B9 and at least one phage that is capable of infecting P. acnes strain PA4. In some embodiments, the mixture comprises a least one phage that is capable of infecting P. acnes strain B9 and at least one phage that is capable of infecting P. acnes strain PA3. In some embodiments, the mixture comprises a least one phage that is capable of infecting P. acnes strain B9 and at least one phage that is capable of infecting P. acnes strain PA5. In some embodiments, the mixture comprises a least one phage that is capable of infecting P. acnes strain PA4 and at least one phage that is capable of infecting P. acnes strain PA3. In some embodiments, the mixture comprises a least one phage that is capable of infecting P. acnes strain PA4 and at least one phage that is capable of infecting P. acnes strain PA5. In some embodiments, the mixture comprises a least one phage that is capable of infecting P. acnes strain PA3 and at least one phage that is capable of infecting P. acnes strain PA5.

In some embodiments, a mixture comprising at least two phages reduces the time to appearance of phage-resistant bacteria as compared to a single phage. See FIG. 9.

In some embodiments, a mixture comprising at least three phages widens the host range as compared to two phages. In some embodiments, the mixture comprises at least one phage that is capable of infecting P. acnes strain B9, at least one phage that is capable of infecting P. acnes strain PA3, and at least one phage that is capable of infecting P. acnes strain PA4. The ability to infect B9, PA3 and PA4 can also be a property of certain bacteriophage mixtures comprising at least 2 phage (e.g., at least PAP-12 and any one of NS19-1, PS7-1, or PA1-4; at least PA1-4 and any one of NS7-1, PA1-9, PA1-14, PA2-7, PAP-1, PAP-4 or PAP-12),

### Bacterial Lysis

In some embodiments, the P. acnes bacteria to be lysed by the bacteriophage provided herein are in/on the skin, eyes or teeth. In some embodiments, the P. acnes bacteria to be lysed by the bacteriophage provided herein are in a biofilm in/on skin, eyes, teeth, or on an implant.

In some embodiments, the bacteriophage provided herein is capable of lysing P. acnes bacteria that are generally thought to be associated with acne and/or biofilms and may be administered to ameliorate the condition or at least one symptom thereof.

### Cosmetic Compositions

Cosmetic compositions comprising the bacteriophage described herein may be used to modulate acne and/or biofilms. Cosmetic compositions comprising one or more bacteriophage, alone or in combination with prophylactic agents, therapeutic agents, and/or and cosmetically acceptable carriers are provided. In certain embodiments, the cosmetic composition comprises two bacteriophages described herein. In other embodiments, the cosmetic composition comprises three or more bacteriophages described herein.

The cosmetic compositions described herein may be formulated in a conventional manner using one or more cosmetically acceptable carriers comprising excipients and auxiliaries, which facilitate processing of the active ingredients into compositions for cosmetic use. Methods of formulating cosmetic compositions are known in the art (see, e.g., “Harry's Cosmeticology, Chemical Publishing Company 9^{th} Ed; Cosmetics: Science and Technology Series: Edward Sagarin, Interscience Publishers). In some embodiments, the cosmetic compositions are subjected to appropriate formulation for topical administration, including but not limited to methods of manufacturing a gel, cream, lotion, face powder and compacts, skin colorant, body powder, face pack and masks, bath oil, bath powder, bath foam, astringent lotion, antiperspirant, preshave and after shave lotion, or cologne. The composition may be administered once or more daily, weekly, or monthly.

### Pharmaceutical Compositions

Pharmaceutical compositions comprising the bacteriophage described herein may be used to modulate acne and/or biofilms. Pharmaceutical compositions comprising one or more bacteriophage, alone or in combination with prophylactic agents, therapeutic agents, and/or and pharmaceutically acceptably carriers are provided. In certain embodiments, the pharmaceutical composition comprises two bacteriophages described herein. In other embodiments, the pharmaceutical composition comprises three or more bacteriophages described herein.

The pharmaceutical compositions described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, PA). In some embodiments, the pharmaceutical compositions are subjected to appropriate formulation for topical administration, including but not limited to methods of manufacturing a gel, cream, paste, ointment, solution, microemulsion, lotion, liquid wash, spray, application stick, cosmetic, dressing, face-wash, soap, powder, spray, capsule, eye drop, eye ointment, eye lotion, solid, a moist sponge wipe or a composition bonded to a solid surface.

The bacteriophage described herein may be formulated into pharmaceutical compositions in any suitable topical dosage form (e.g., a gel, cream, paste, ointment, solution, microemulsion, lotion, liquid wash, spray, application stick, cosmetic, dressing, face-wash, soap, powder, spray, capsule, eye drop, eye ointment, eye lotion, solid, a moist sponge wipe or may be bonded to a solid surface.) and for any suitable type of administration (e.g., immediate-release, pulsatile-release, delayed-release, extended-release or sustained release). In some embodiments, the bacteriophages are formulated for administration as a gel, cream, paste, ointment, solution, microemulsion, lotion, liquid wash, spray, application stick, cosmetic, dressing, face-wash, soap, powder, spray, capsule, eye drop, eye ointment, eye lotion, solid, a moist sponge wipe or may be bonded to a solid surface. The composition may be administered once or more daily, weekly, or monthly.

In some embodiments, the bacteriophage may be covalently attached to a carrier particle, for use as a topical formulation or for application to an implant. In some embodiments, the carrier particle is typically approximately spherical, may have an average diameter of up to 20 microns, up to 15 microns, up to 10 microns, from 0.1 microns, from 0.5 microns or any combinations of these—e.g. from 0.1 microns to 20 microns or from 0.5 microns to 10 microns. The particles in general can be approximately round or spheroid; they are preferably smooth, especially for use on sensitive parts of the body. Particle size is suitably measured using methods and apparatus recognized as standard in the art. Particle sizing in dispersions can be accomplished using a variety of techniques, including laser diffraction, dynamic light scattering (DLS), disc centrifugation, and light microscopy. Examples of sizing equipment are made by Malvern Instruments (UK), using laser diffraction methods. In some embodiments, bacteriophages may be covalently attached to a plurality of particles. These are preferably in relatively homogenous form, in which a large proportion of the plurality of particles have diameters of up to 20 microns, up to 15 microns, up to 10 microns, from 0.1 microns, from 0.5 microns or any combinations of these—e.g. from 0.1 microns to 20 microns or from 0.5 microns to 10 microns. In some embodiments, 80% or more, 90% or more or 95% or more of the particles with phage covalently attached have diameters of up to 20 microns, up to 15 microns, up to 10 microns, from 0.1 microns, from 0.5 microns or any combinations of these—e.g. from 0.1 microns to 20 microns or from 0.5 microns to 10 microns. WO2015118150 describes further the carrier particle that may be used for the bacteriophage formulation.

Particles for use in the application to which bacteriophage are immobilized by covalent bonding are generally substantially inert to the animal to be treated. In examples, nylon particles (beads) were used. Other inert, preferably non-toxic biocompatible material may be used. In addition, the particle may be made of a biodegradable material. Suitable materials include polymethyl methacrylate, polyethylene, ethylene/acrylate copolymer, nylon-12, polyurethane, silicone resin, silica and nylon 1010. WO2003093462 describes further materials that the particles may be made from.

Immobilization or attachment of bacteriophage to the particle substrate may be achieved by covalent bonds formed between the bacteriophage coat protein and the carrier substrate. Bacteriophage may also be immobilized to the substrate via their head, tail, or capsule by activating the substrate particle before the addition and bonding of bacteriophage. The term “activated/activating/activation” refers to the activation of the substrate such as electrically, e.g. by corona discharge, or by reacting said substrate with various chemical groups (leaving a surface chemistry able to bind viruses, such as bacteriophage head, tail or capsule group). WO2015118150, WO2003093462 and WO2007072049 describe further the activation of said substrate, coupling of phage to substrate, and details of methods for covalent attachment of phage to particles.

In some embodiments, the bacteriophage is formulated for delivery to mammalian skin, eyes, teeth, or implant. In some embodiments, the composition comprises the bacteriophage and a pharmaceutically or cosmetically acceptable excipient, wherein the bacteriophage and the excipient do not occur together in nature. In some embodiments, the composition comprises the bacteriophage and a pharmaceutically or cosmetically acceptable excipient, wherein the excipient is a non-naturally occurring excipient. In some embodiments, the composition comprises the bacteriophage encapsulated in a pharmaceutically or cosmetically acceptable polymer, wherein the polymer is a non-naturally occurring polymer. In some embodiments, the composition described herein may be encapsulated to facilitate a longer shelf life and storage of phage to ensure reproducible dosages, and to facilitate effective delivery to the desired site of action or adsorption. In some embodiments, the composition may be encapsulated in emulsions, ointments, polymeric or lipid microparticles (microspheres & microcrystals), nanoparticles, nanofibers, microfibers, membranes, thin film structures and/or liposomes. Natural and synthetic polymers may be used for phage encapsulation. Phage encapsulation may be performed using a variety of hydrophilic and hydrophobic polymers including but not limited to agarose, alginate, chitosan, pectin, whey protein, gelled milk protein, hyaluronic acid methacrylate, hydroxypropyl methyl cellulose (HPMC), poly(N-isopropylacrylamide), Poly(DL-lactide:glycolide), polyesteramide, polyvinyl pyrrolidone, polyethylene oxide/polyvinyl alcohol, cellulose diacetate, and/or polymethyl methacrylate. Examples of the materials that could be used for preparation of phages encapsulated in liposomes include, but are not limited to, phosphatidylcholine, cholesterol, Softisan 100™; soybean phosphatidylcholine, DOPC (1,2-dioleoyl-sn-glycero-3-phosphocholine), DOPS (1,2-dioleoyl-sn-glycero-3-phospho-L-serine), DLPC (1,2-Dilauroyl-sn-glycero-3-phosphorylcholine), Cholesterol-PEG 600, and/or cholesteryl esters. Solid-liquid particles for topical administration can be produced by solid lipids and adjuvants including, but not limited to, surfactants and emulsifiers, e.g., stearic acid, oleic acid, tripalmitin, cetyl alcohol, cetyl palmitate, tristearin, trimyristin, and hydrogenated vegetable fat (HVF), glyceryl behenate, glyceryl monostearate, glyceryl palmitostearate, glyceryl tripalmitate, sodium taurocholate, octadecyl alcohol, Tween 80, Poloxamer 188, Compritol® 888 ATO, Imwitor® 900, Precirol® ATO5, carnauba wax and isodecyl oleate, hydrogenate phosphatidylcholine, cholesterol. Malik et al., 2017; Bacteriophages Methods and Protocols 2018; and Das and Chaudhury, 2011 describe further materials and techniques for bacteriophage encapsulation.

In some embodiments, the composition is in single dosage form. Single dosage forms may be in a liquid, gel or cream form. Single dosage forms may be administered directly to a patient without modification or may be diluted or reconstituted prior to administration. Single dosage forms of the composition may be prepared by portioning the composition into smaller aliquots, single dose containers, single dose liquid forms, or single dose solid forms, such as tablets, granulates, nanoparticles, nanocapsules, microcapsules, microtablets, pellets, or powders. A single dose in a solid form may be reconstituted by adding liquid, typically sterile water or saline solution, or mixing with other dermal formulation components, prior to administration to a patient.

Dosage regimens may be adjusted to provide a therapeutic response. Dosing can depend on several factors, including severity and responsiveness of the disease, route of administration, time course of treatment (days to months to years), and time to amelioration of the condition. For example, a single bolus may be administered at one time, several divided doses may be administered over a predetermined period of time, or the dose may be reduced or increased as indicated by the therapeutic situation.

In some embodiments, the ingredients are supplied either separately or mixed together in unit dosage form. The pharmaceutical compositions may be packaged in a hermetically sealed container such as an ampoule or sachet indicating the quantity of the agent. In some embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In some embodiments, one or more of the prophylactic or therapeutic agents or pharmaceutical compositions is supplied as a dry sterile lyophilized powder in a hermetically sealed container and reconstituted prior to administration. In some embodiments, the dry sterilized lyophilized powder is produced by spray-drying and can include a mixture of one of the following: 30-50% dextran, 40-70% sucrose, 0.5-2% tris, and 1-3% leucin; or 30-50% hydroxyethyl starch, 40-70% sucrose, 0.5-2% tris, and 1-3% leucine. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants.

### Methods of Treatment

Methods of treating acne in a mammal using a bacteriophage, a bacteriophage mixture, and/or a composition comprising a bacteriophage or a bacteriophage mixture are provided. In some embodiments, a method of treating acne comprises administering to a subject the P. acnes bacteriophage described herein. In some embodiments, a method of treating or preventing acne comprises administering to a subject the P. acnes bacteriophage described herein. In some embodiments, a method of reducing the amount of P. acnes comprises administering to a subject the P. acnes bacteriophage described herein. In some embodiments, a method of treating or preventing the development of biofilms in/on skin, eyes or teeth comprises administering to a subject the P. acnes bacteriophage described herein. In some embodiments, a method of preventing the development of biofilms on implants comprises administering to a subject the P. acnes bacteriophage described herein.

In some embodiments, a method of treating acne comprises administering to a mammal determined to have a skin disease associated with one or more strains of P. acnes bacteria, the bacteriophage mixture, and/or the composition comprising a bacteriophage mixture described herein. In some embodiments, a method of preventing acne comprises administering to a mammal at risk of acquiring a skin disease associated with one or more strains of P. acnes bacteria, the bacteriophage mixture, and/or the composition comprising a bacteriophage mixture described herein. In some embodiments, a method of treating biofilms comprises administering to a mammal determined to have a skin disease associated with one or more strains of P. acnes bacteria, the bacteriophage mixture, and/or the composition comprising a bacteriophage mixture described herein. In some embodiments, a method of preventing biofilms on implants comprises applying on the implant at risk of being colonized with P. acnes bacteria, the bacteriophage mixture, and/or the composition comprising a bacteriophage mixture described herein.

In some embodiments, the bacteriophage and the method of treatment is used prophylactically. For example, the method comprises administering to a mammal determined to be susceptible to a skin disease associated with one or more strains of P. acnes bacteria, the bacteriophage mixture, and/or the composition comprising a bacteriophage or a bacteriophage mixture described herein. In another example, the method comprises applying on an implant determined to be susceptible to P. acnes colonization, the bacteriophage mixture, and/or the composition comprising a bacteriophage or a bacteriophage mixture described herein. In some embodiments, an implant is determined to be susceptible to P. acnes colonization based on the reported occurrence in the scientific literature of colonization on such an implant.

In some embodiments, the bacteriophage is administered more than once to achieve a desired therapeutic effect. For example, when a host bacterium is destroyed, the bacteriophage that infected said bacterium can no longer multiply because its host has been eradicated and may be eliminated from the skin, eyes, teeth, or implant, and the bacteriophage may need to be re-administered, e.g., at least twice daily, at least daily, at least weekly, or at least monthly.

In some embodiments, the phage described herein may be administered in combination with one or more known and suitable medicaments for acne, including one or more topical or oral agents selected from a list comprising an antibiotic, an anti-comedonal, an anti-P. acnes agent, an anti-inflammatory, an anti-seborrhoeic agent, an anti P. acnes vaccine, a keratolytic agent, a sebum penetration enhancer, and a sunscreen. In that context, the one or more topical or oral agents and the phage may be administered simultaneously, or sequentially with the bacteriophage composition, i.e., in a single formulation or in separate formulations packaged either together or individually. In some embodiments, the keratolytic agent or the sebum penetration enhancer may be a cleansing agent that opens pores or enhances sebum penetration, which agent is administered prior to administering the phage.

### Methods of Selecting a Composition of the Application

Methods of selecting a mixture of phages that can treat a subject are provided herein. In some embodiments, the method of selecting a mixture of phages that can treat a subject by the methods set forth herein comprises (1) obtaining a biological sample from the subject, e.g., from the skin, eyes, teeth (2) culturing the bacteria obtained from the biological sample, (3) inoculating the cultured bacteria with a mixture of bacteriophage described herein, and (4) determining the amount (percentage) of P. acnes bacteria in the sample that are lysed by the mixture. In some embodiments, lysis of at least 50%, 60%, 70%, 80%, 85%, 90%, 95%, 97%, 98%, 99% or more of the P. acnes bacteria in the sample indicate that the mixture of phage can be used to treat the subject. In some embodiments, the lysis is determined by a liquid media assay and/or a solid media assay as described herein.

Methods of determining whether P. acnes is lysed by the phage mixture are provided herein. In some embodiments, the method comprises identifying P. acnes in a biological sample by culturing the sample in the presence of 20 μg/mL furazolidone, which selects for Propionobacterium, checking each bacterial colony by 16S ribosomal RNA gene (rRNA) sequencing to differentiate P. acnes from other Propionobacterium species and determining the percentage sensitivity of the P. acnes to the phage mixture, before and after phage treatment. In preferred embodiments, the percentage sensitivity of P. acnes in the sample to phage treatment is determined by using quantitative PCR (qPCR), with primers specific for P. acnes bacteria, before and after phage treatment. In some embodiments, the percentage sensitivity of P. acnes in the sample to phage treatment is determined by deep sequencing followed by metagenomic analysis, before and after phage treatment.

In some embodiments, the biological sample includes but is not limited to a skin sample, swab, a skin biopsy, a skin scraping, pus, wound, an abscess. In some embodiments, determining whether the cultured bacteria are lysed by the bacteriophage comprises performing direct test for phage sensitivity using the plaque assay or liquid OD method described herein. In some embodiments, a positive sample or subject to be treated is determined by the presence of plaque in a plaque assay. In some embodiments, a positive sample or subject to be treated is determined by a reduction in OD in a liquid OD assay. In some embodiments, the phage is labeled with a detectable marker, e.g., a luminescent or other marker that is activated upon infection, and the infection of the bacteria is determined by detecting an increase in the marker, e.g., in a luminescence assay.

### EXAMPLES

### Example 1. Sample Sourcing and Processing for Bacteriophage

### Materials and Methods

The candidate bacteriophages are isolated from sewage samples. Batches of sewage that comprise 5-6 raw sewage samples of 400 mL each, obtained from different places at different times are centrifuged and the supernatant is filtered sequentially through Merck (Merck KGaA, Darmstadt, Germany) Millipore glass fiber prefilter APFD, followed by prefilter APFB followed by Express plus PES 47 mm disks 0.45 μm filters using a vacuum filtration system. The pooled sewage sample mixes are concentrated using a Merck Millipore 100 kDa PelliconXL filter system (from 2L to 20 mL) (Merck KGaA, Darmstadt, Germany). Concentrated sewage samples are then filtered through a 0.451 μm filter and stored at 4° C. Thus, each final sewage phage sample comprises of bacteriophages from 5-6 samples of different geographical origins.

### Example 2. P. acnes Bacterial Hosts Used in the Isolation, Production and Host-Range Experiments

### Materials and Methods

The following strains of P. acnes are either ordered or isolated in-house. Strains originating from the culture collections are revived per their instructions and aliquoted and preserved at −80° C. PA4 and PAP are laboratory isolates, purified by three serial rounds of colony isolation and confirmed to be P. acnes by 16S Sanger sequencing. In addition, all strains including both commercial and isolated ones are sequenced and their identity as P. acnes checked by aligning whole genome sequencing reads to the reference genome of P. acnes KPA171202 or a published genome if available. Table 2 below shows the sources of the P. acnes strains.

TABLE 2
Sources of P. acnes strains

| | Isolated | | Bacterial bank |
|---|---|---|---|
| Strain | from | Identifier | references and sources |
| PA1 | Human | 417/52 | ATCC 11828; BCRC 16146; |
| | subcutaneous | [VPI 0391] | CCUG 6369; KCTC 3320; |
| | abscess | | VPI 0391; JCM 6473 |
| PA2 | Human wound | Derm-CH102 | ATCC 33179; BCRC 16149 |
| PA3 | Human forehead | 3 | ATCC 29399; BCRC 16148 |
| PA4 | Culture | BiomX internal | DSM 32714, deposited on |
| | contamination | strain PA4 | Dec. 5, 2017 |
| PA5 | Culture | VPI 9 | ATCC 51277 |
| | contamination | | |
| PA6 | Clinical case of | NCTC 5235 | ATCC 6923; BCRC 16145; |
| | acne vulgaris | | NCTC 5235 |
| PA7 | Unknown | NCTC 556 | ATCC 6922; BCRC 16144; |
| | | [VPI 4978] | NCTC 556 |
| PA8 | Clinical case of | NCTC 4311 | ATCC 6921; BCRC 16150; |
| | acne vulgaris | [NCTC 2083a] | NCTC 4311 |
| PA9 | Unknown | 643-C, | ATCC 12930; NTC 10390 |
| | | [NCTC 10390] | |
| PA10 | Facial acne | NCTC 737 | ATCC 6919; BCRC 10723; |
| | | [VPI 0389] | CCUG 1794; CECT 5684; |
| | | | CGMCC 1.5003; CIP 53.117; |
| | | | DSM 1897; KCTC 3314; |
| | | | KCTC 5008; LMG 16711; |
| | | | LMG 3591; NBRC 107605; |
| | | | NCTC 737; NRRL B-4224; |
| | | | VKM Ac-1450; VPI 0389; |
| | | | JCM 6425 |
| PA11 | Unknown | Gerath [23, C-7, | ATCC 11827; KCTC 5012 |
| | | VPI 4979] | |
| PA13 | Culture | KPA171202 | DSM 16379 |
| | contamination | | |
| PAP | Culture | | DSM 32709, deposited on |
| | contamination | | Dec. 5, 2017 |

### Example 3. Screening Environmental and Clinical Samples for Phage that Recognize P. acnes

### Materials and Methods

Screening of the concentrated sewage samples for P. acnes phage is performed using a modified spot/drop plaque assay (Mazzocco A., et al. (2009) as described. Briefly, after overnight growth in Brain Heart Infusion broth (Becton, Dickinson and Company, Franklin Lakes, New Jersey) supplemented with 5 g/L Yeast Extract (Acros Organics, Geel, Belgium) (“BHIS”), the strains are diluted 1:3 in BHIS. About 5.5 hours after the dilution, the strains are centrifuged (2000×g, 5 min, room temp) and re-suspended in 200 μL BHIS. In parallel, 48-well plates containing 0.5 mL per well of 1.5% agar BHIS (“bottom agar”) are prepared and incubated in a Don Whitley A35 anaerobic chamber (Don Whitley Scientific, Shipley, UK) for at least 2 hours to remove traces of oxygen from the medium. Aliquots of 0.4% agar BHIS (“top agar”) are melted and inserted into a heat-block at 55 degrees inside the anaerobic workstation. A volume of 100 μL of each of the concentrated P. acnes cultures is mixed with 4 mL top agar and 75 μL of this mixture is dispensed onto the bottom agar in the appropriate wells. The plates are left at room temperature for 20 minutes to solidify, then incubated for 40 minutes at 37° C. to recover.

After recovery, 10 μL of each of the sewage samples is pipetted onto the appropriate wells, left to absorb for 20 minutes, then incubated inverted overnight at 37° C. When plaques are observed, these are isolated by picking individual, well isolated plaques (using a sterile pipetting tip) into phage buffer (Tris-HCL pH 7.5 50 mM, NaCl 100 mM, MgCl₂·6H₂O 5 mM, MnCl₂·4H₂O 0.1 mM in DDW) and the process is repeated an additional 2 times for a total of 3 rounds of isolation.

Phage stocks of the phages isolated by the methods described above are created on the bacterial host PA1 by mixing 4 mL of BHIS with 400 μL from an overnight 3 mL cultures of PA1. The culture is incubated anaerobically at 37° C. for 5 hours after which 50 μL of each phage isolate is added and this culture is further incubated overnight. The following morning, the cultures are centrifuged (4500×g, 10 min, 4° C.) and filtered through a 0.45 μm filter and stored at 4° C. Plaque assays are carried out as described above to estimate the titer of each phage stock.

### Results

A total of 21 phages are isolated by the methods described above. Phages PA1-4, PA1-9, PA1-11, PA1-12, PA1-13, PA1-14 are isolated on PA1. Phages NS19-1, NS13, NS7-1, PA2-4, PA2-7, PA2-13 are isolated on PA2. Phages PS7-1, PAP-1, PAP-4, PAP-7, PAP-8, PAP-11, PAP-12, PAP-13, PAP-14 are isolated on PAP.

### Example 4. Host Range Analysis of Isolated Phage

### Materials and Methods

Host range analysis for phage isolated on the PA1, PA2, and PAP strains, is performed on twelve P. acnes strains: PA1, PA2, PA3, PA4, PAP, PA5, PA6, PA7, PA8, PA9, PA10, and PA11. Each phage is added (10 μL) to bacterial lawns of P. acnes strains, in 48 well plates by drop assay. Plates are incubated for 24 hrs (37° C.) in anaerobic conditions, after which plaques become visible on the bacterial lawns. Plates with bacterial lawns of PA1, PA2, and PAP, and their respective phage serve as positive control. Host range analysis is done for each of the phage using 10 μL containing 10⁴ phage per well.

### Results

As shown in FIG. 1, where +++ indicates that the plaques are too numerous to count or total clearing, ++ indicates a countable number of plaques greater than 10, + indicates 1 to 10 plaques and − indicates no visible plaques, most P. acnes bacteria are very sensitive to a large number of the newly isolated phage.

### Example 5. Validation of Wide Phage Host Range Against Clinical P. acnes Strains that Demonstrate Antibiotic Resistance

Isolation of Clinical P. acnes Strains

A range of P. acnes strains (PAC1-PAC6 and PAC12-PAC14) are isolated from skin samples of healthy volunteers by using Hygiena Deep cleansing nose strips (Beautycare E.G., Bnei Darom, Israel) applied to the nose. A range of P. acnes strains (2001-1, 2001-3, 2001-5, 2002-3, 2002-7, 2002-9, 2003-2, 2003-8, 2003-10, 2004-8_2, 2004-8_3, 2004-84, 2004-8_5, 2004-8_6, and 2004-8_10) are isolated from skin samples of acne patients by using Hygiena Deep cleansing nose strips applied to the nose. The nose strips are applied according to the manufacturer's supplied User Guide on volunteer's noses (10 minutes, on wetted nose). Next, the strip is carefully removed from the nose and inserted into a polypropylene tube with 3 ml anacrobized BHIS broth. The tube is scaled and pulse vortexed for 15 seconds, and then inserted into the anaerobic work station. Using sterile tweezers, the nose strip is removed from the tube and discarded. A volume of 50 μL of the BHIS tube sample is then smeared using a sterile Drigalski spatula on a BHIS agar petri dish and incubated overnight at 37° C. in the anaerobic work station incubator. The following morning, using a sterile loop (for each colony), several separate colonies are picked and re-plated onto a new BHIS agar plate. The plate is incubated at 37° C. in the anaerobic work station incubator. The taxonomy of each isolate is confirmed to be P. acnes by 16S sequencing. Frozen stocks are prepared for each strain by freezing a stationary culture in 20% glycerol and stored at −80° C. Host range experiments on these strains are performed by plaque assay as described above, using 10 μL containing 10⁴ phage per well. Table 3 below shows the list of clinical isolates of P. acnes.

TABLE 3
List of clinical isolates of P. acnes

| Name | Isolated from |
|---|---|
| PAC1 | Healthy volunteer |
| PAC2 | Healthy volunteer |
| PAC3 | Healthy volunteer |
| PAC5 | Healthy volunteer |
| PAC6 | Healthy volunteer |
| PAC12 | Healthy volunteer |
| PAC13 | Healthy volunteer |
| PAC14 | Healthy volunteer |
| 2001-1 | Acne patient |
| 2001-3 | Acne patient |
| 2001-5 | Acne patient |
| 2002-3 | Acne patient |
| 2002-7 | Acne patient |
| 2002-9 | Acne patient |
| 2003-2 | Acne patient |
| 2003-8 | Acne patient |
| 2003-10 | Acne patient |
| 2004-8_2 | Acne patient |
| 2004-8_3 | Acne patient |
| 2004-8_4 | Acne patient |
| 2004-8_5 | Acne patient |
| 2004-8_6 | Acne patient |
| 2004-8_10 | Acne patient |

Host-Range of Phage on a Subset of the Clinical Isolates of P. acnes

Host range analysis for the phage isolated on the PA1, PA2, and PAP strains, is performed on a subset of the clinical P. acnes strains. See FIG. 2. Each phage is added (10 μL) to bacterial lawns of P. acnes strains, in 48 well plates by drop assay. Plates are incubated overnight (37° C.) in anaerobic conditions, after which plaques become visible on the bacterial lawns. Host range analysis is done for each of the phage using 10 μL containing 10⁴ phage per well. As can be seen in FIG. 2, where +++ indicates that the plaques are too numerous to count or total clearing, ++ indicates a countable number of plaques greater than 10, + indicates 1 to 10 plaques, indicates no visible plaques, NT indicates Not Tested, most clinical P. acnes bacteria are very sensitive to a large number of the different phage strains.

### Assaying of Phage or Antibiotic Sensitivity

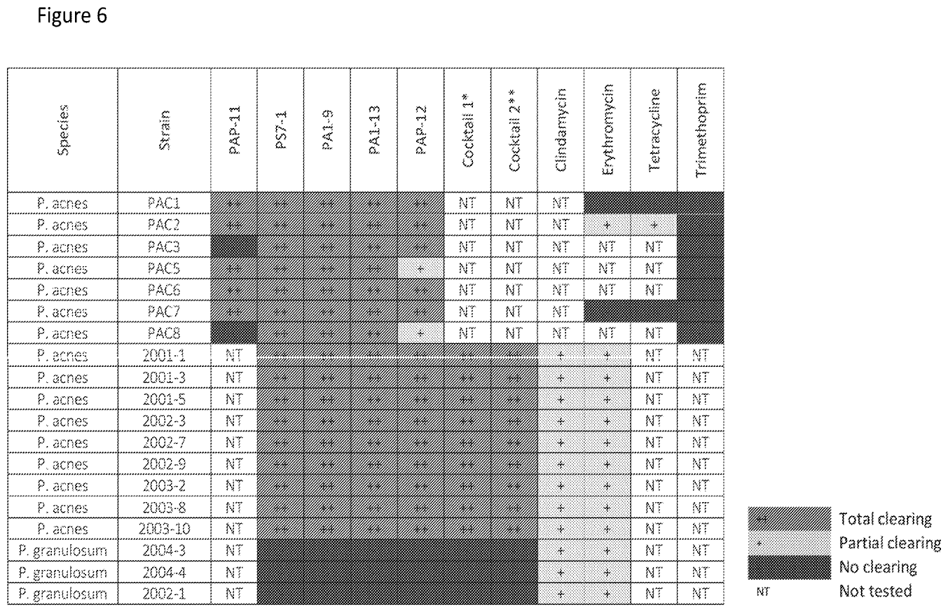

The phage sensitivity of the bacterial strains is tested either by regular plaque assay as described previously, or by dropping 5 μL of a stock of the phage on a lawn of the tested bacteria and letting the plate incubate for 24 hours at 37° C. anaerobically. The antibiotic susceptibility of the strains is measured by placing 5-10 μL of the antibiotics on a freshly prepared lawn of the bacterial strain, and incubating overnight at 37° C. anaerobically. The concentrations of clinically relevant antibiotics used are 15 μg/mL Trimethoprim (Merck KGaA, Darmstadt, Germany), 20 μg/mL Erythromycin (Acros Organics, Geel, Belgium), 1% Clindamycin (Selleck Chemicals, Houston, Texas) and 10 μg/mL Tetracycline (Merck KGaA, Darmstadt, Germany). See FIG. 6, where ++ indicates that the plaques are totally cleared, + indicates partially cleared, − indicates not cleared, and NT indicates Not Tested. Results of host range studies on the clinical P. acnes strains suggest that wide coverage of infectivity of multiple P. acnes strains can be attained by application of individual phage or phage mixtures comprising different phage with overlapping host infectivity patterns.

### Example 5. Non-Infectivity of P. granulosum Strain PAC4

### Materials and Methods

In order to examine the specificity of the isolated phage against other bacterial strains, they are tested for their ability to infect a close relative of P. acnes which is also found in the skin microbiome, P. granulosum.

As part of the clinical P. acnes strain isolation study as described in Example 4, some strains of P. granulosum were isolated from skin samples of healthy volunteers by using nose pore strips Hygiena Deep cleansing nose strips (Beautycare E.G., Bnei Darom, Israel) applied to the nose. Strain assignment is done by 16S sequencing followed by validation using whole genome sequencing. Using the previously mentioned plaque assay, the infectivity of P. granulosum strain PAC4 is tested for the whole panel of P. acnes phage at a concentration of 10⁶ PFU/mL.

### Results

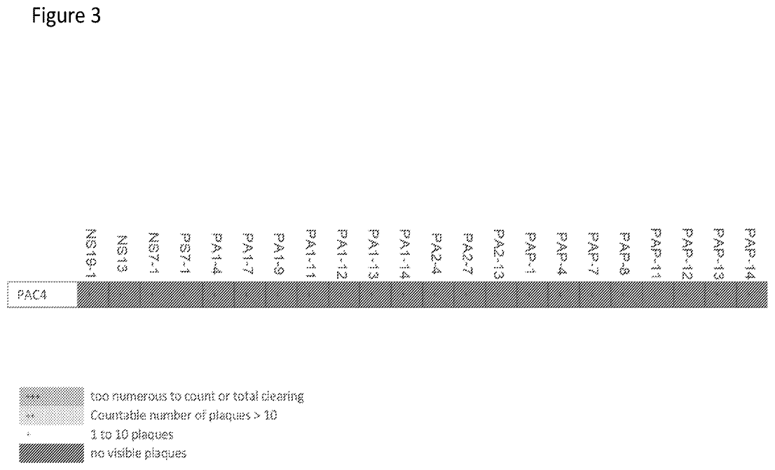

As can be seen in FIG. 3, no plaque or clearing zone is observed in any of the plates/wells which further supports the notion that P. acnes phage are species specific.

To further confirm these results, 3 more clinical isolates of P. granulosum (2004-8, 2004-4 and 2002-1) are tested with four P. acnes phage (PS7-1, PA1-9, PA1-13, PAP-12) by spot/drop plaque assay at a concentration of 10⁶ PFU/mL using 5 μL drops. These bacterial strains show sensitivity to Clindamycin (1% gel, ˜10 μL) and Erythromycin (20 μg/mL, 5 μL) when the antibiotics are applied directly to a bacterial lawn of these strains, but are resistant to all the tested phage.

### Example 6. Phage Activity Against a P. acnes Mutant Strain that has Developed Phage Resistance

### Developing a Phage Resistant Mutant

To develop phage resistant mutants, PA3 is cultured overnight anaerobically at 37° C. in a 4 mL BHIS culture tube, diluted the morning after 1:10 and grown a few hours to OD 0.4-0.8. The culture is then further diluted to OD 0.2. The diluted culture is dispensed at 0.2 mL per well into a 96-well plate, after which 10 μL of phage PAP-1 at 10⁶ PFU/mL is added and finally this is covered with 40-50 μL of mineral oil. After sealing the plate with a sterile SealPlate film (Merck KGaA, Darmstadt, Germany) the plate is incubated in a Tecan M200 Pro plate reader (Tecan Group, Männedorf, Switzerland) at 37° C. to follow the infection dynamics. Following lysis by the phage, the OD decreases. Later, the OD starts increasing because of growth of resistant mutants. A fresh BHIS agar plate is then inoculated with 100 μL of the culture in the wells showing re-growth, and incubated for 48 hours anaerobically at 37° C. This mutant PA3 strain that has become resistant to the PAP-1 phage is named B9.

### Corroborating PAP-1 Resistance of B9

Colonies of B9 are used to inoculate 4 mL of BHIS and incubated for 48 hours due to slower growth than the PA3 strain. These are then diluted to OD 0.2 and tested using the same method as described above. Growth of the mutants is identical with or without the addition of high titer phage PAP-1, while the wildtype (WT) control shows lysis by the phage and lowering of OD compared to the WT control without phage, supporting the fact that these are indeed P. acnes mutants resistant to PAP-1 even though derived from a parent strain that is sensitive to this phage.

### Sequencing of Resistant Mutants

The same culture that is used to re-test the mutants is further divided into two, with approximately 2 mL being mixed 1:1 with 50% glycerol and frozen at −80° C. to create a stock. The remaining culture is spun down and genomic DNA is extracted using the QIAGEN QIAamp DNA mini kit (Qiagen N.V., Hilden, Germany), by following the manufacturer's protocol for DNA isolation from gram positive bacteria. This DNA is sequenced using an Illumina MiSeq machine (Illumina, San Diego, California). Analysis of the sequencing results shows that the mutant is indeed P. acnes strain PAP3 with various modifications along the genome.

### Testing the Sensitivity/Resistance of Mutant B9 to Other Phage

To test the resistance of B9 against the various P. acnes phage, a 4 mL tube of BHIS is inoculated with B9 colonies and incubated over 3 days at 37° C. These are then spun down by centrifugation (5 min, 2000×g, RT) and re-suspended in 0.2 mL BHIS. This culture is then used as the basis for a plaque assay using the method described previously.

As can be seen in FIG. 4, where R indicates resistance to infection with the tested phage, and S indicates sensitivity to infection with the tested phage, the B9 mutant which is isolated based on resistance to PAP-1 has simultaneously developed resistance to additional phage to which the original PA3 is sensitive. There are four phage which display the ability to infect the mutant bacterial strain (NS19-1, PS7-1, NS13 and PA1-4) and an additional phage (PA1-13) with very limited infectivity.

Phage that Infect the B9 Mutant but not its Parent PA3

When testing the parent strain of the mutant B9, named PA3, using the plaque assay described above, several phage tested show borderline (1-10 plaques, phages NS19-1, PS7-1, PA2-13, PAP-7, PAP-11) or no infectivity (no visible plaques, phages NS13, PA1-4, PA24, PAP-8) at a titer of 10⁶ PFU/mL. Interestingly, some of the phages with borderline infectivity of strain PA3, namely phages NS19-1 and PS7-1, showed infectivity of the more phage resistant mutant B9. Phages NS19-1 and PS7-1 have more than 89.5% genomic similarity while phages NS13 and PA1-4 which are able to infect B9 but not the parent strain PA3 are also closely related at more than 91.5% genomic similarity. See FIG. 10.

### Example 7. Non-Interference Between Phage in Host Infection

### Materials and Methods

Host strain PA4, which is efficiently infected only by phages PA1-4 and PAP-12, is used to test whether there is any negative effect on mixing different P. acnes phage together. Strain PA4 is grown overnight in 4 mL BHIS. The following morning, it is diluted 10× in BHIS and allowed to grow until an OD of ˜0.8. This culture is used as the basis for doing a 48-well plaque assay as described previously. After the 48-well plates containing both bottom and top agar with bacteria are prepared, all of the phages, including PAP-12, are diluted to 10⁶ PFU/mL. These diluted phages are then mixed either in a 1:1 ratio with fresh BHIS medium or 1:1 with the 10⁶ PFU/mL stock of PAP-12. 10 μL of the phage+BHIS combination or phage+PAP-12 combination is used in the plaque assay. After the required overnight incubation, plaques are counted.

### Results

As can be seen in FIG. 5, the presence of additional phage did not interfere with the PAP-12 effect on PA4 suggesting that several phages can be combined in a mixture without impairing the specific function of any individual phage.

### Example 8. Efficacy of Phage In Vivo in a Mouse Ear P. acnes Induced Swelling Model

### Intradermal Study

### Materials and Methods

The intradermal study consisted of three groups ICR female mice (n=20) (ENVIGO CRS, Ness Ziona, Israel). All groups are subjected to model induction by a single intradermal (ID) injection of 20 μL P. acnes suspension at a concentration of 1×10¹⁰ CFU/mL into the dorsum of the right car. In Group 2, 20 μL of PAP-7 phage suspension is injected intradermally into the same ear, 3 hours post P. acnes injection. In Group 3, 20 μL of phage suspension is injected intradermally to the same ear 3 hours prior to P. acnes injection. In all groups, the left ear serves as a control and is injected with PBS once in Group 1 or twice in Groups 2 and 3, under identical experimental conditions. Group 1 serves as a control group for model induction.

Ear thickness is measured by micro-caliper (Mitutoya, 0.01 mm (Mitutoyo Europe GmbH, Neuss, Germany) shortly before model induction, 24 hours, 48 hours and 72 hours post model induction. The increase in ear thickness of the right ear (i.e. P. acnes injected ear) is calculated as a percentage of the left ear (i.e. PBS injected ear).

### Results

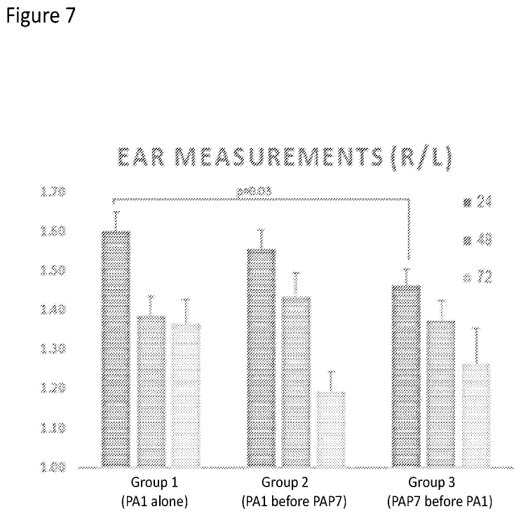

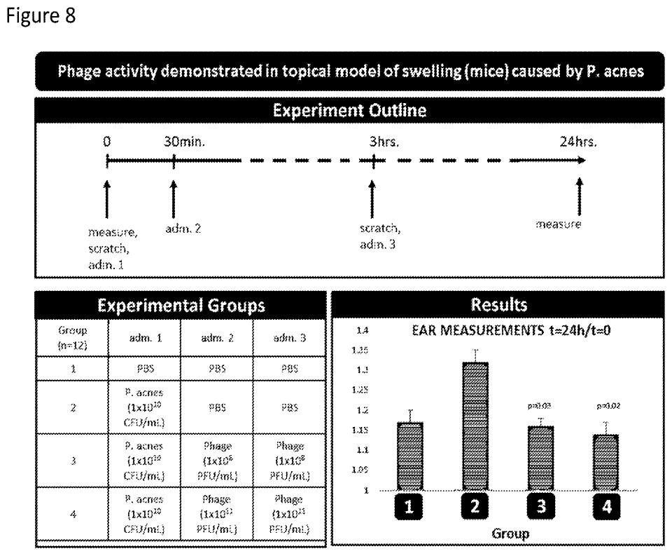

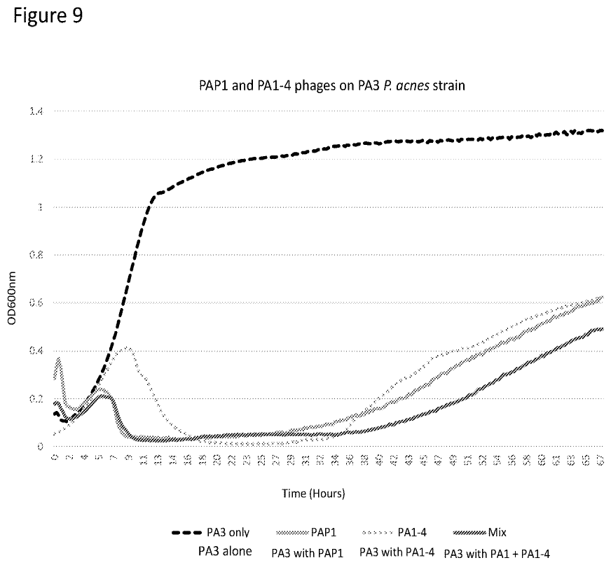

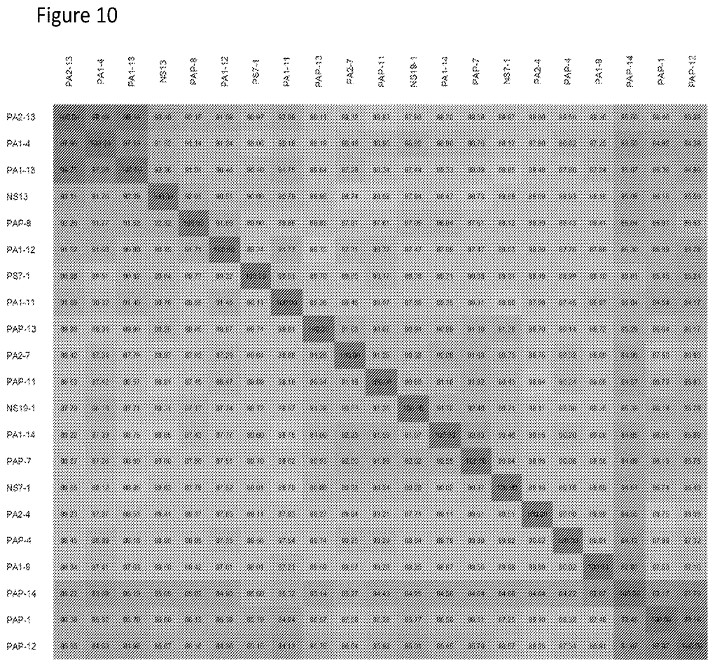

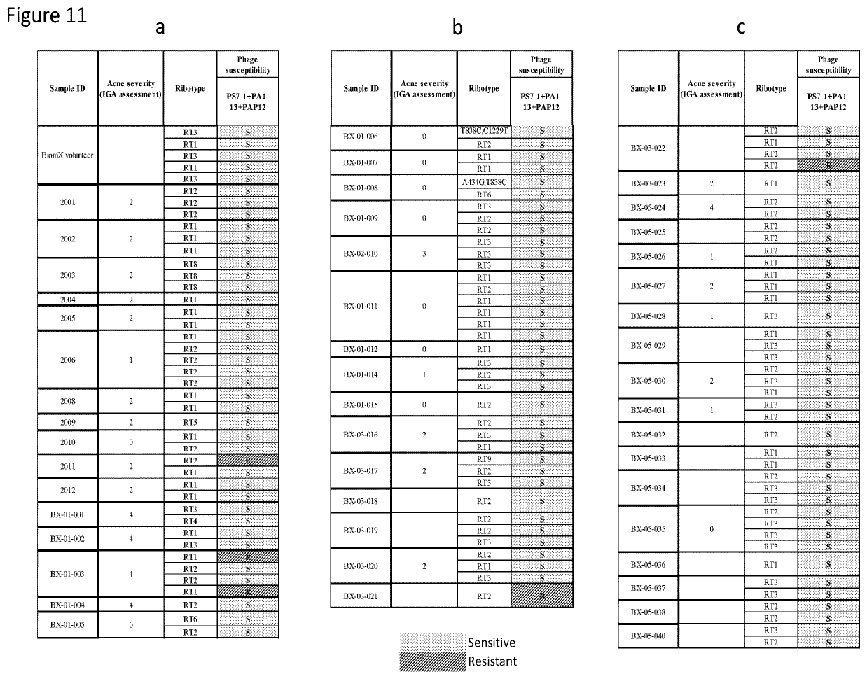

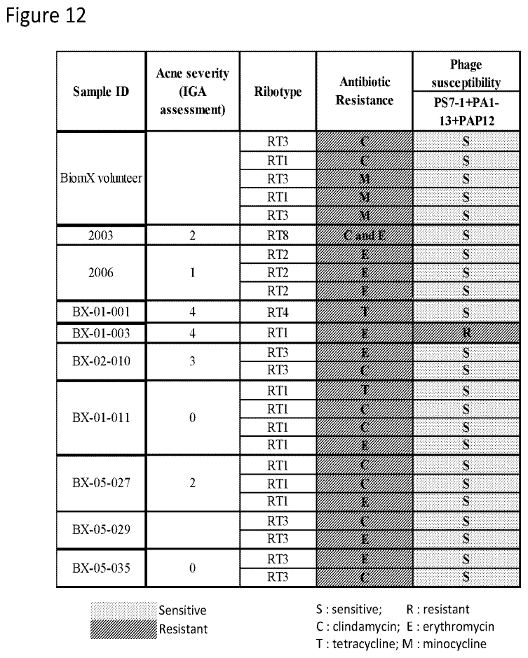

FIG. 7 shows the reduction of ear swelling after treatment with phage in the P. acnes intradermal infection model, 24 hours, 48 hours and 72 hours post model induction. Intradermal phage administration prevents ear swelling in the mouse model when ear thickening is induced by intradermal injection of P. acnes. This effect is demonstrated by car thickening mostly after 24 hrs from P. acnes induction.

### Topical Study

### Materials and Methods

The topical study consists of four groups of n=6 ICR female mice (ENVIGO CRS, Ness Ziona, Israel). All groups are subjected to model induction by gentle scratching with a 0.2 ml tip (10 times/ear) to generate a single superficial laceration. The laceration causes the skin to become visibly damaged, characterized by reddening with no indication of bleeding. Immediately following the laceration, 10 μL of a single topical application of a 1×10¹⁰ CFU/mL phage combination of PS7-1, PAP-1, and PAP-12 (Groups 2,3 and 4) or PBS (Group 1) is applied. Both ears are treated. Treatment is at 30 min and 3 hours after infection with P. acnes. Group 1 and 2 receive PBS, Group 3 receives 1×10⁸ CFU/mL phage suspension, and Group 4 receives 1×10¹¹ CFU/mL phage suspension. Both ears of all animals are measured using a micro-caliper (Mitutoya, 0.01 mm) (Mitutoyo Europe GmbH, Neuss, Germany) before model induction and at 24 hours following model induction.

### Results

Using this model, the efficacy of topical administration of P. acnes phage when administered after P. acnes in the bacterial induced ear swelling model is demonstrated. As can be seen in FIG. 8, at the 24-hour time point, there is no swelling in the P. acnes+phage treated ears (Groups 3 and 4) as compared to the P. acnes only treated ears (Group 2). It can also be seen that administration of phage at the lower titer (Group 3) approximating the anticipated therapeutic dose brings about the entire effect and a higher titer is not required.

### Example 9. Efficacy of Isolated Phage on Biofilm

### Materials and Methods

To test our phage against P. acnes biofilms, the following experiment is performed on biofilms produced in the laboratory. An overnight culture of PA1 is diluted 1:1 with fresh BHIS and incubated for 4 hours at 37° C. after which the culture is once again diluted 1:1 this time with anacrobized BHIS supplemented with 1% glucose (final conc. 0.5%). Next, a 96-well plate (Thermo Fisher Scientific, Waltham, Massachusetts) is seeded with 200 μL of the diluted culture per well. The wells are sealed using 8-well strip caps (Thermo Fisher Scientific, Waltham, Massachusetts). This is incubated for 3 days to let the biofilm form. After 72 hours, 10 μL of either mixture 1 (PAP-12, PA1-9 and PA1-13), mixture 2 (PA7-1, PA1-9 and PA1-13), mixture 3 (PAP-12, PA1-9, PA1-13 and PS7-1) or 2 μL of Erythromycin is added to the relevant wells. The amount of phage added per mixture corresponds to 10⁵ total PFU while for the antibiotic Erythromycin, a final concentration of 100 μg/mL (enough to inhibit PA1 growth when not in a biofilm) is used.

At 24 hrs or 48 hrs post-treatment depending on the wells, the medium is carefully removed from each well and the remaining biofilm is picked from each well into a separate Eppendorf tube using a sterile tip. At this point, 1 mL of fresh BHIS is added to the biofilm tubes, sonicated to remove the phage inside for 5 minutes (Elmasonic S 100, at 37 kHz) (Elma Schmidbauer GmbH (Singen, Germany), and centrifuged (4 min, 4000 rpm, 25° C.). The supernatant containing the phage is carefully removed to a clean tube for quantification and the process was repeated a total of 3 times to make sure all phage are removed.

To quantify the surviving bacteria, the pellet is re-suspended in 100 μL BHIS, 10-fold serially diluted in BHIS and plated on BHIS agar plates. The plates are incubated for 72 hrs at 37° C. anaerobically after which the colonies are counted and results rounded to the nearest log. In parallel, the phage quantities inside the biofilm are tested by taking the supernatant set aside earlier, and using the aforementioned plaque assay to assess the original quantities.

### Results

TABLE 4
Remaining log bacteria after phage or antibiotic treatment of biofilm

| | Logs CFU | |
|---|---|---|
| | 24 hours post treatment | 48 hours post treatment |
| No treatment (n = 6) | More than 9 | More than 9 |
| Phage mixture 1 (n = 3) | 4 | 4 |
| Phage mixture 2 (n = 3) | 4 | 4 |
| Phage mixture 3 (n = 3) | 4 | 6 |
| Erythromycin (n = 3) | More than 9 | 8 |

As can be seen in Table 4, a large decrease of at least 4 logs in bacterial counts after 24 hours with phage is seen compared to no visible decrease in the antibiotic arm. After 48 hours the two 3-phage mixtures still show 4 logs less CFU than the no treatment arm, while the 4-phage mixture shows 6 logs less CFU. This is in contrast to the antibiotic arm of the experiment that showed 8 logs of remaining bacteria per milliliter. These results demonstrate the efficacy of phage against target P. acnes bacteria even when these are growing in biofilm form. By comparison, the antibiotic crythromycin, to which this bacterial strain is sensitive, is significantly less effective in lysing P. acnes bacteria growing as biofilm. The amount of phage remaining inside the biofilm is 5.3×10⁵, 7.0×10⁵ and 4.3×10⁵ per well for phage mixtures 1, 2 and 3 respectively.

### Example 10. Applying Phage Combinations Increases the Time Until Mutants Appear

### Materials and Methods

The application of a combination of P. acnes infecting phage is tested for its ability to delay the time until appearance of a resistant mutant P. acnes strain. When infecting with single phage, bacteria with mutations in the phage receptor tend to be selected for. By combining phage with different receptors or different receptor affinities, the time to mutant (TTM, the time it takes for a bacterial mutant to take over the culture) can be increased. The two phages to be combined, PAP1 and PA1-4, are selected because they differ both genetically and functionally based on host-range. PAP1 belongs to the functional cluster 3b while PA1-4 belongs to the functional cluster 5. Phages are tested separately and in a mixture on PA3 bacteria using OD₆₀₀ measurements every 15 minutes as described previously. The individual phages and the phage mixture are tested at a final concentration of 10⁶ PFU/mL.

All work is performed at anaerobic conditions in an A35 Don Whitley anaerobic workstation. PA3 is cultured overnight anaerobically at 37° C. in a 4 mL BHIS culture tube, diluted the morning after 1:3 and incubated for 4 hours until reaching OD 0.8-1.2. In the meanwhile, phages PAP-1 and PA1-4 are diluted to 10⁸ PFU/mL, and a 1:1 mixture of these phage at this concentration is also prepared. The culture is then further diluted to OD 0.2. This is dispensed at 190 μL per well into a 96-well plate, after which 10 μL of the phage containing samples ire added in triplicates. Finally, this is covered with 40 μL of mineral oil. After sealing the plate with a sterile SealPlate film (Merck KGaA, Darmstadt, Germany), the plate is incubated in a Tecan M200 Pro plate reader at 37° C. to follow the infection dynamics with measurements every 15 minutes. Following lysis by the phage, the OD decreases. Later, the OD increases because of the growth of resistant mutants.

### Results

All cultures containing phage show an initial expected decrease in OD due to phage predation. To compare the time it takes for the resistant mutant bacteria to appear, the OD₆₀₀ threshold of 0.2 is used to measure the TTM. As can be seen in FIG. 9, the PAP-1 and PA1-4 only cultures show TTMs of 42 and 40 hours while the 1:1 mixture mix of the two phages shows a TTM of 50 hours. These results demonstrating a shift in the time to the appearance of resistant mutants when both phage are applied simultaneously support the previous observations that these two phage belong to different functional clusters.

### Example 11. Genetic Analyses of Phage Shows High Levels of Similarity Between Phage with Some Small Diversity

### Sequencing of Phage Stocks

Phage DNA is extracted from 200 μL of stock containing at least 10⁹ PFU. Bacterial DNA present in each of the lysates is removed by treating with the Ambion Turbo free DNA kit (Thermo Fisher Scientific, Waltham, Massachusetts) after which the QIAGEN QIAamp DNA mini kit (Qiagen N.V., Hilden, Germany) is used to extract the gDNA of the phage per manufacturer instructions. Elution of DNA is done by two rounds of elution with 30 μL AE buffer incubated for 3-5 minutes on the spin column and DNA is quantified using a Nanodrop 2000. After extraction, Illumina sequencing libraries are created following the protocol of Baym et al. (Baym et al., 2015), and sequenced using a MiSeq machine.

### Phage DNA Assembly and Analysis

Reads are assembled using SPAdes v3.10.1 (Nurk et al., 2013). The phage genomes are compared by using BLASTN (Altschul et al., 1997), by combining all non-overlapping alignment segments (BLAST HSPs), summing their numbers of identical matches and dividing this sum with the length of the longer sequence to get to the percent identity. Clustering of the percent identity table is done by the Hclust algorithm in R-studio version 1.0.143 (R Core Team (2017). R: A language and environment for statistical computing. R Foundation for Statistical Computing, Vienna, Austria). The results are shown in Table 5 below and FIG. 10. As shown in FIG. 10, there are two small groups with very high genomic similarity, namely group 1 (PA1-13, PA2-13 and PA1-4) and group 2 (PAP-1 and PAP-12).

TABLE 5
Characterization of isolated phage

| Name | Isolated on | Length (kbp) | GC % |
|---|---|---|---|
| NS19-1 | PA2 | 30.1 | 53.9 |
| NS13 | PA2 | 29.7 | 54.5 |
| NS7-1 | PA2 | 29.7 | 54.1 |
| PS7-1 | PA2 | 29.9 | 54.2 |
| PA1-4 | PA1 | 29.1 | 54.5 |
| PA1-9 | PA1 | 29.3 | 54.0 |
| PA1-11 | PA1 | 29.5 | 54.1 |
| PA1-12 | PA1 | 29.2 | 54.1 |
| PA1-13 | PA1 | 29.9 | 54.4 |
| PA1-14 | PA1 | 29.8 | 54.2 |
| PA2-4 | PA2 | 29.6 | 54.3 |
| PA2-7 | PA2 | 29.7 | 54.5 |
| PA2-13 | PA2 | 29.7 | 54.4 |
| PAP-1 | PAP | 29.6 | 54.0 |
| PAP-4 | PAP | 29.8 | 54.1 |
| PAP-7 | PAP | 29.9 | 54.3 |
| PAP-8 | PAP | 29.4 | 54.6 |
| PAP-11 | PAP | 29.9 | 54.3 |
| PAP-12 | PAP | 29.6 | 54.0 |
| PAP-13 | PAP | 30.0 | 54.4 |
| PAP-14 | PAP | 30.3 | 54.5 |

### Example 12. Efficacy of Therapeutic Cocktail on Clinical P. acnes Strains

### Bacterial Isolation

P. acnes was isolated from healthy volunteers and individuals with acne according to protocol MBC-CL-01-2016 (clinicaltrials.gov identifier NCT03009903). Helsinki IRB approvals were obtained for both protocols and informed consent signed by all volunteers. Collection and isolation of P. acnes from skin was obtained either from the nose, using cosmetic nose strips (Beautycare E.G., Bnei Darom, Israel) applied per manufacturer's instructions or from the forehead, by rubbing sterile dry swabs (COPAN diagnostics) moistened in sampling buffer (0.1% Triton+0.075M phosphate buffer) for 30 seconds on a 4 cm² area.

The obtained nose strips were cut in two and one half placed in bacterial growth medium and the second half in phage buffer. The bacterial medium was checked for the presence of P. acnes bacteria by plating onto supportive agar plates containing furazolidone for Propionibacteria isolation and followed for colony growth. Colonies were picked, named and tested by molecular biology techniques for identity. Species assignment of the bacteria was done by 16S rDNA sequencing followed by validation using whole genome sequencing.

Alternatively, two skin samples were obtained from each individual's forehead: one swab was stored in an empty sterile tube at −80° C. for DNA extraction. The other swab used placed in sterile 15 mL test tube containing 4 mL of 0.1% Triton+0.075M phosphate buffer and stored at 4° C. and used for bacterial isolation. Each swab was vortexed for 30 seconds and 10 μl of each sample was streaked onto BHIS agar plate containing 20 μg/mL Furazolidone, to select for Propionibacteria. Isolated propionibacterium colonies were cultured in BHIS under anaerobic conditions and glycerol stocks were prepared. Equal volumes of overnight culture were mixed with 50% glycerol, divided into cryotubes and stored at −80° C.

### Determination of Antibiotic Resistance:

For the isolation of antibiotic resistant colonies, 50 μl out of the 4 ml derived from the skin swabs were spread evenly onto plates to which antibiotics were added as follows: 0.5 μg/mL Clindamycin; BHIS+20 μg/mL Furazolidone+0.5 μg/mL Erythromycin; BHIS+20 μg/mL Furazolidone+5 μg/mL Tetracycline; BHIS+20 μg/mL Furazolidone+5 μg/mL Minocycline. All antibiotics were purchased from Sigma-Aldrich (Rehovot, Israel).

Plates were allowed to dry in a biological hood for −30 minutes and then incubated in a lock & lock box with three GasPak Easy sachets (Becton Dickinson, Franklin Lakes, New Jersey) under anaerobic conditions, at 37° C. for approximately one week. Plates were inspected daily for colony appearance. When bacterial growth was visible, a total of 10 individual colonies from each sample were picked (different morphologies and different antibiotics were chosen).

The antibiotic susceptibility of the clinical isolates derived from volunteers with and without acne was further evaluated by prepared lawns of the selected bacterial strains and adding 5-10 μL drops of the selected relevant antibiotics onto the fresh lawns and observing for clearance after overnight incubation at 37° C. under anaerobic conditions. Clinically relevant antibiotics were used at the following concentrations: (0.5 μg/ml Clindamycin (Selleck Chemicals, Houston, Texas), 5 μg/ml Minocycline (Sigma-Aldrich (Rehovot, Israel), 0.5 μg/ml Erythromycin (Acros Organics, Geel, Belgium), 5 μg/ml Tetracycline (Merck KGaA, Darmstadt, Germany)). The results of the antibiotic resistance of the clinical strains are summarized in FIG. 12.

### Determination of Phage Sensitivity

In order to test sensitivity to the phage cocktail (PS7-1, PA1-13 and PAP-12), the chosen isolated bacterial colonies were grown overnight in 4 mL BHIS at 37° C. in the anaerobic workstation. The next morning, each bacterial strain culture was diluted in BHIS to a starting OD of ˜0.7 and incubated at 37° C. in the anaerobic workstation.

When an increase in bacterial OD was observed, samples were tested either by preparing a phage cocktail lawn at a concentration of 10⁶ and plating 5 μl bacterial strain on it, or by preparing a lawn of each strain onto BHIS plates, drying in the biological hood for ˜30 minutes and addition of 5 μl of phage cocktail at a final concentration of 10⁴ to each lawn. Plates were left to dry for additional 30 minutes in the biological hood and then incubated at 37° C. in the anaerobic workstation for ˜48 h. The results of the phage infection are summarized in FIG. 11.

### Determination of Ribotype

To determine the isolates' ribotype, 16s gene PCR analysis was performed using primers 27F and 1492R, analyzed by Sanger sequencing and aligned against RT1 ribotype with CloneManage software.

In order to obtain overlapping sequences around the ends of the reads and prevent mistakes due to sequencing errors, three primers were used (27F, 529F, 1492R) for sequencing. The primers' sequences are as follows in Table 6:

TABLE 6
Primer sequence identification numbers used to
identify ribotypes of the bacterial isolates.

| SEQ ID | | |
|---|---|---|
| NO. | Brief Description | Size (base pairs) |
| 22 | 27F - | 20 |
| | AGAGTTTGATCCTGGCTCAG | |
| 23 | 1492R - | 20 |
| | CGGTTACCTTGTTACGACTT | |
| 24 | 529F - | 20 |
| | AGCGTTGTCCGGATTTATTG | |

The relevant ribotype was determined according to the alignment to RT1 sequence (shown below) and the ribotype specific mutations as seen in Table 7:

TABLE 7
Ribotype mutations compared to RT1

| Ribotype | Mutations compared to RT1 |
|---|---|
| RT1 | — |
| (SEQ ID NO: 25) | |
| RT2 | T838C |
| RT3 | T992C |
| RT4 | G1043C, A1187C |
| RT5 | G1043C |
| RT6 | T838C, C1322T |
| RT7 | G529A |
| RT8 | G989A, T992C |
| RT9 | G1254A |
| RT10 | T539C, G1043C |

Approximately 130 clinical lines were tested, and most were identified as ribotypes RT1, RT2 or RT3. The four ribotypes generally thought to be most associated with acne in patients (RT4, RT5, RT8 and RT9) were also found and determined to be susceptible to the phage cocktail (see FIGS. 11 and 12).

### Results

A three-phage cocktail targets over 95% of clinical P. acnes strains (sec FIG. 11). Approximately 130 P. acnes clinical lines were isolated from acne patients and healthy individuals as described above. Of these, a large number exhibited antibiotic resistance against one or more antibiotics when tested against a panel of 4 antibiotics (FIG. 12). Only 5 samples were found to be resistant to a three-phage cocktail that has been designed based on the principles described above. A few isolates that were found to be resistant to furazolidone and resistant to the phage cocktail, were later identified by sequencing as not being P. acnes isolates (data not shown). When a subset of the P. acnes clinical lines was subjected to ribotyping, it was further observed that the phage susceptible clinical lines included strains of the four ribotypes generally considered to be associated with acne. These are ribotypes RT4, RT5, RT8 and RT9. These results demonstrate susceptibility of clinical strains with the generally considered disease-associated ribotypes to a designed phage cocktail.

### REFERENCES

1. Abedon, S. T., and Yin, J. (2009). Bacteriophage plaques: theory and analysis. Methods in molecular biology (Clifton, N.J.), 501, 161-74.
2. Abedon, S. T., et al. (2011). Bacteriophage prehistory: is or is not Hankin, 1896, a phage reference? Bacteriophage 1, 174-178.
3. Achermann Y et al. (2014). Propionibacterium acnes: from Commensal to Opportunistic Biofilm-Associated Implant Pathogen. Clinical Microbiology Reviews, 27(3):419-440.
4. Altschul S. F. et al. (1997) Gapped BLAST and PSI-BLAST: a new generation of protein database search programs. Nucleic Acids Res. 25:3389-3402.
5. Archer N. K., et al. (2011). Staphylococcus aureus biofilms: properties, regulation, and roles in human. Virulence 2:445-459.
6. Aucoin P. J. et al. (1986). Intracranial pressure monitors. Epidemiologic study of risk factors and infections. American Journal of Medicine 80(3):369-376.
7. Baym M. et al. (2015). Inexpensive multiplexed library preparation for megabase-sized genomes” PLoS ONE, 10(5), 1-15.
8. Brandwein, M. et al. (2016). Microbial biofilms and the human skin microbiome. NPJ Biofilms and Microbiomes, 2, 3.
9. Chaudhury A, and Das S. (2011). Recent advancement of chitosan-based nanoparticles for oral controlled delivery of insulin and other therapeutic agents. AAPS PharmSciTech. 2011 Mar; 12(1):10-20.
10. Clokie M. R., Kropinski A. M. (eds) Bacteriophages. Methods in Molecular Biology™, Vol 501. 81-85 Humana Press.
11. Clokie M. R. J. et al. (2018). Bacteriophages Methods and Protocols Volume 3, Humana Press
12. Conen, A et al. (2008). Characteristics and treatment outcome of cerebrospinal fluid shunt-associated infections in adults: a retrospective analysis over an 11-year period. Clinical Infectious Diseases, vol. 47, no. 1, pp. 73-82.
13. Costerton J. W. et al., (1999). Bacterial biofilms: a common cause of persistent infections. Science 284:1318-1322.
14. Cove, J. H. et al. (1983). Effects of oxygen concentration on biomass production, maximum specific growth rate and extracellular enzyme production by three species of cutaneous propionibacteria grown in continuous culture. Journal of General Microbiology 129(11):3327-3334.
15. Dunne W. M. (2002). Bacterial adhesion: seen any good biofilms lately? Clin. Microbiol. Rev. 15:155-166.
16. Del Pozo J. L. et al. (2009). Pilot study of association of bacteria on breast implants with capsular contracture. Journal of Clinical Microbiology, vol. 47, no. 5, pp. 1333-1337.
17. Del Pozo J. L. and Patel R (2009). Infection associated with prosthetic joints. The New England Journal of Medicine, 361(8):787-794.
18. Delahaye F. et al. (2005). Propionibacterium acnes infective endocarditis. Study of 11 cases and review of literature,” Archives des Maladies du Coeur et des Vaisseaux, vol. 98, no. 12, pp. 1212 1218.
19. Delgado, S. et al. (2011). Identification, typing and characterisation of Propionibacterium strains from healthy mucosa of the human stomach. International Journal of Food Microbiology 149(1):65-72.
20. Deramo V. A. et al. (2001). Treatment of Propionibacterium acnes endophthalmitis. Current Opinion in Ophthalmology, vol. 12, no. 3, pp. 225-229.
21. Dodson, C. C. et al. (2010). Propionibacterium acnes infection after shoulder arthroplasty: a diagnostic challenge,” Journal of Shoulder and Elbow Surgery, 19(2):303-307.
22. Dréno, B. et al. (2016). Bacteriological resistance in acne: a call to action. Eur. J. Dermatol. 26, 127-132.
23. Fischer, N. et al. (2013). Deciphering the intracellular fate of Propionibacterium acnes in macrophages. BioMed Research International, 2013, [603046].
24. Fitz-Gibbon, S et al. (2013). Propionibacterium acnes Strain Populations in the Human Skin Microbiome Associated with Acne, Journal of Investigative Dermatology, 133(9): 2152-2160.
25. Golkar, Z. et al. (2014). Bacteriophage therapy: a potential solution for the antibiotic resistance crisis. J. Infect. Dev. Ctries 8, 129-136.
26. Gribbon E. M. et al. (1994). The microacrophily and photosensitivity of Propionibacterium acnes. Journal of Applied Bacteriology 77(5):583-590.
27. Guio L et al. (2009). Chronic prosthetic valve endocarditis due to Propionibacterium acnes: an unexpected cause of prosthetic valve dysfunction. Rev Esp Cardiol. 62(2):167-77.
28. Haidar R et al. (2010). Propionibacterium acnes causing delayed postoperative spine infection: review,” Scandinavian Journal of Infectious Diseases, vol. 42, no. 6-7, pp. 405-411.
29. Hannigan G. D. and Grice E. A. (2013). Microbial Ecology of the Skin in the Era of Metagenomics and Molecular Microbiology. Cold Spring Harb Perspect Med, 3:a015362.
30. Hoefnagel D. et al. (2008). Risk factors for infections related to external ventricular drainage. Acta Neurochirurgica, 150(3) 209-214.
31. Holmberg, A. et al. (2009). Biofilm formation by Propionibacterium acnes is a characteristic of invasive isolates. Clinical Microbiology and Infection, 15: 787-795.
32. Human Microbiome Project Consortium, (2012). Structure, function and diversity of the healthy human microbiome. Nature, 486:207-214.
33. Hunyadkurti, J. et al. (2011). Complete genome sequence of Propionibacterium acnes type IB strain 6609. Journal of Bacteriology 193(17):4561-4562.
34. Hyman, P., and Abedon, S. T. (2010). Bacteriophage host range and bacterial resistance. Advances in applied microbiology (1st ed., Vol. 70, pp. 217-48). Elsevier Inc.
35. Jończyk-Matysiak, E. et al. (2017). Prospects of Phage Application in the Treatment of Acne Caused by Propionibacterium acnes. Frontiers in Microbiology, 8:1-11.
36. Kurtz S. M. et al. (2012). Economic burden of periprosthetic joint infection in the United States. J Arthroplasty. 27(8 Suppl): 61-5.el.
37. Kutter, E. (2009). Phage host range and efficiency of plating. Methods in molecular biology (Clifton, N.J.), 501, 141-9.
38. Levy R. M., et al. (2003). Effect of antibiotics on the oropharyngeal flora in patients with acne. Arch. Dermatol. 139, 467-471.
39. Lyke, K. E. et al. (2001). Ventriculitis complicating use of intraventricular catheters in adult neurosurgical patients. Clinical Infectious Diseases, 33(12):2028-2033.
40. Malik D. J., et al. (2017). Formulation, stabilisation and encapsulation of bacteriophage for phage therapy. Adv Colloid Interface Sci. 49:100-133.
41. Marinelli L. J. et al. (2012). Propionibacterium acnes bacteriophages display limited genetic diversity and broad killing activity against bacterial skin isolates. mBio 3(5):e00279-12
42. Mayhall, C. G. et al. (1984). Ventriculostomy-related infections. A positive epidemiologic study. The New England Journal of Medicine 310(9):553-559.
43. Mazzocco A., et al. (2009). Enumeration of Bacteriophages Using the Small Drop Plaque Assay System.
44. McDowell A. et al. (2008). A new phylogenetic group of Propionibacterium acnes. J. Med. Microbiol. 57:218-224.
45. Michalek S. et al. (2015). The use of trimethoprim and sulfamethoxazole (TMP-SMX) in dermatology. Folia Med. Cracov. 55, 35-41.
46. Nishijima K. et al. (1996). Sensitivity of Propionibacterium acnes isolated from acne patients: comparative study of antimicrobial agents. J. Int. Med. Res. 24, 473-477.
47. Nurk et al., (2013). Assembling Single-Cell Genomes and Mini-Metagenomes From Chimeric MDA Products. Journal of Computational Biology. 20(10):714-737.
48. Perry A. and Lambert P. (2011). Propionibacterium acnes: infection beyond the skin.
49. Piper K. E. (2009) Microbiologic diagnosis of prosthetic shoulder infection by use of implant sonication. Journal of Clinical Microbiology, vol. 47, no. 6, pp. 1878-1884.
50. Portillo, M. E. et al. (2013). Propionibacterium acnes: An Underestimated Pathogen in Implant-Associated Infections,” BioMed Research International, vol. 2013, Article ID 804391, 10 pages.
51. Rieger, U. M. et al. (2009). Sonication of removed breast implants for improved detection of subclinical infection. Aesthetic Plastic Surgery, vol. 33, no. 3, pp. 404-408.
52. Sampedro M. F. et al. (2010). A biofilm approach to detect bacteria on removed spinal implants. Spine (Phila Pa 1976) 20; 35(12):1218-24.
53. Saper D., et al. (2015). Management of Propionibacterium acnes infection after shoulder surgery Curr Rev Musculoskelet Med. 8(1): 67-74.
54. Sardana K. et al. (2016). A cross-sectional pilot study of antibiotic resistance in Propionibacterium acnes strains in Indian acne patients using 16s-RNA polymerase chain reaction: a comparison among treatment modalities including antibiotics, benzoyl peroxide, and isotretinoin. Indian J. Dermatol. 61, 45-52.
55. Schafer P. et al. (2008). Prolonged bacterial culture to identify late periprosthetic joint infection: a promising strategy. Clin. Infect. Dis. 47:1403-1409.
56. Söderquist B. et al. (2010). Propionibacterium acnes as an etiological agent of arthroplastic and osteosynthetic infections—two cases with specific clinical presentation including formation of draining fistulae. Anaerobe 16(3):304-6.
57. Stirling A. et al. (2001). Association between sciatica and Propionibacterium acnes. Lancet, vol. 357, no. 9273, pp. 2024-2025.
58. Sulakvelidze et al. (2001). Bacteriophage therapy. Antimicrob. Agents Chemother. 45, 649-659.
59. Tan, J. K. L. and Bhate, K. (2015). A global perspective on the epidemiology of acne. Br J Dermatol, 172: 3-12.
60. Trampuz, A. et al. (2007). Sonication of removed hip and knee prostheses for diagnosis of infection. The New England Journal of Medicine 357(7):654 663.
61. Tyner H. and Patel R. (2016). Propionibacterium acnes: from Commensal to Opportunistic Biofilm-Associated Implant Pathogen Anaerobe 40:63-67.
62. Walsh T. R. et al. (2016). Systematic review of antibiotic resistance in acne: an increasing topical and oral threat. Lancet Infect. Dis. 16, e23-e33.
63. Zeller V. et al. (2007). Propionibacterium acnes: an agent of prosthetic joint infection and colonization J Infect. 55(2):119-24.
64. International Publication No. WO2015118150 Treatment of topical and systemic bacterial infections
65. International Publication No. WO2003093462 Immobilisation and stabilisation of virus
66. International Publication No. WO2007072049 Particle binding

1. (canceled)

2. The method of claim 14, wherein:
the first bacteriophage infects and lyses P. acnes strain B9; and
the second bacteriophage infects and lyses P. acnes strain PA4,
wherein the two bacteriophages have different lytic specificities from one another with respect to the P. acnes strains B9 and PA4.

3. The method of claim 14, wherein:
the first bacteriophage infects and lyses P. acnes strain PA3; and
the second bacteriophage infects and lyses P. acnes strain B9,
wherein the two bacteriophages have different lytic specificities from one another with respect to the P. acnes strains PA3 and B9.

4. The method of claim 14, wherein:
the first bacteriophage infects and lyses P. acnes strain PA3; and
the second bacteriophage infects and lyses P. acnes strain PA4,
wherein the two bacteriophages have different lytic specificities from one another with respect to the P. acnes strains PA3 and PA4.

5. The method of claim 14, wherein:
the first bacteriophage infects and lyses P. acnes strain PA4; and
the second bacteriophage infects and lyses P. acnes strain PA5,
wherein the two bacteriophages have different lytic specificities from one another with respect to the P. acnes strains PA4 and PA5.

6. The method of claim 14, wherein the composition comprises at least one phage that infects and lyses at least one P. acnes strain selected from the group consisting of: PA1, PA2, PA6, PA7, PA8, PA9, PA10, PA11, and PAP.

7. The method of claim 14, wherein in toto the bacteriophages in the composition infect and lyse each of P. acnes strains PAI, PA2, PA6, PA7, PA8, PA9, PAIO, PAI 1, and PAP.

8. The method of claim 14, wherein the composition comprises:
a. a first bacteriophage that infects and lyses at least one P. acnes strain selected from the group consisting of: B9, PA4, PA3, and PA5;
b. a second bacteriophage that infects and lyses at least one P. acnes strain selected from the group consisting of: B9, PA4, PA3, and PA5; and
c. a third bacteriophage that infects and lyses at least one P. acnes strain selected from the group consisting of: B9, PA4, PA3, and PA5;
wherein the at least one P. acnes strain infected by the second bacteriophage is not infected and lysed by at least one of the first bacteriophage and the third bacteriophage, and
wherein the at least one P. acnes strain infected by the third bacteriophage is not infected and lysed by at least one of the first bacteriophage and the second bacteriophage.

9. The method of claim 8, wherein:
the first bacteriophage infects and lyses P. acnes strain PA3;
the second bacteriophage infects and lyses P. acnes strain PA4; and
the third bacteriophage infects and lyses P. acnes strain B9, and,
wherein each of the three bacteriophages have different lytic specificities from one another with respect to the P. acnes strains PA3, PA4 and B9.

10. The method of claim 9, wherein the composition comprises at least three bacteriophages selected from the group consisting of: PS7-1, PA1-11, PAP-12, PA1-9 and PA1-13.

11. The method of claim 14, wherein the composition is formulated for delivery to mammalian skin or mammalian eyes.

12. The method of claim 11, wherein the composition is formulated for topical application.

13. The method of claim 12, wherein the composition is in the form of a gel, a cream, anointment, a lotion, a paste, a solution, a microemulsion, a liquid wash, a spray, an application stick, a cosmetic, a dressing, a face-wash, a soap, a powder, a spray, a capsule, an eye drop, an eye ointment, an eye lotion, a solid, or a moist sponge wipe, or is bonded to a solid surface.

14. A method for treating or preventing acne in a mammalian subject in need or at risk thereof, the method comprising administering to the subject a composition comprising:
a first bacteriophage that infects and lyses at least one Propionibacterium acnes (P. acnes) strain selected from the group consisting of B9, PA4, PA3, and PA5;
a second bacteriophage that infects and lyses at least one P. acnes strain selected from the group consisting of B9, PA4, PA3, and PA5 that is not infected and lysed by the first bacteriophage; and a pharmaceutically or cosmetically acceptable adjuvant, carrier or vehicle.

15. The method of claim 14, comprising the additional step of administering to the subject one or more topical or oral agents comprising an antibiotic, an anti-comedonal, an anti-P. acnes agent other than a bacteriophage, an anti-inflammatory, an anti-seborrheic agent, and/or a sunscreen.

16. The method of claim 15, wherein the one or more topical or oral agents further include a keratolytic agent and/or a sebum penetration enhancer.

17. The method of claim 15, wherein:
the antibiotic comprises an antibiotic gel, an antibiotic cream, an antibiotic lotion, and/or an oral antibiotic;
the anti-comedonal comprises a retinoid, an azelaic acid, and/or an isotretinoin;
the anti-P. acnes agent other than a bacteriophage comprises benzoyl peroxide, dapsone, azelaic acid, erythromycin, tetracycline and clindamycin, sodium sulfacetamide, adapalene, minocycline, trimethoprim, nadifloxacin, ofloxacin, doxycycline, ampicillin, cephalexin, gentamycin, and/or trimethoprimsulfamethoxazole;
the anti-inflammatory comprises tetracycline, erythromycin, clindamycin, nicotinamide, minocycline, trimethoprim and/or isotretinoin; and/or
the anti-seborrheic agent comprises spironolactone, Dianette™ (cyproterone acetate and ethinylestradiol) and/or isotretinoin.

18. The method of claim 16, wherein the keratolytic agent comprises glycolic acid, lactic acid, mandelic acid, hydroxycapric acid, phytic acid, malic acid, citric acid, tartaric acid, salicylic acid, urea, and/or sulfur.

19. The method of claim 16, wherein the sebum penetration enhancer comprises a polysorbate surfactant, a non-ionic surfactant, an unsaturated fatty acid, an unsaturated alcohol, an aliphatic alcohol, a transcutol, a phospholipid, an unsaturated triglyceride, propylene glycol, and/or dipropylene glycol.

20. The method of claim 14, wherein the composition is administered every 12 or 24 hours at a dose of 10⁵ to 10¹³ plaque forming units (PFU).

21. The method of claim 14, wherein the composition is administered every 48 or 72 hours at a dose of 10⁵ to 10¹³ plaque forming units (PFU).

22. The method of claim 20, wherein the composition is administered every 12 or 24 hours at a dose of 10⁷ to 10¹¹ PFU.

23. The method of claim 21, wherein the composition is administered every 48 or 72 hours at a dose of 10⁷ to 10¹¹ PFU.

24. The method of claim 20, wherein the composition is administered at a dose of 10⁶ to 10¹¹ PFU.

25. The method of claim 20, wherein the composition is administered at a dose of 10⁷ to 10⁹ PFU.

26. The method of claim 14, wherein the acne is acne vulgaris, acne conglobata, acne fulminans, Hidradentis suppurativa, scalp acne, acne associated with Progressive Macular Hypomelanosis, acne associated with SAPHO syndrome, or acne associated with Fatal Bacterial Granuloma after Trauma.

27. The method of claim 21, wherein the composition is administered at a dose of 10⁶ to 10¹¹ PFU.

28. The method of claim 21, wherein the composition is administered at a dose of 10⁷ to 10⁹ PFU.
